SEQUENCE LISTING (2 sequences)

SEQ ID NO: 1 - DNA, 1699 bp, homo sapien
  CDS (274)..(891)
cttgcctgcaaacctttacttctgaaatgacttccacggctgggacgggaaccttccacccacagctatgcctctgattggtgaatggtgaaggtgcctgtctaacttttctgtaaaaagaacacacgcctccagacagccagccctcaagcatcacatacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacaacacgagttgccggctgctgcagcaggagggtctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtcttctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactca

SEQ ID NO: 2 - protein, 206 aa, homo sapien
MNFQQRLQSLWTLARPFCPPLLATASQMQMVVLPCLGFTLLLWSQVSGAQGQEFHFGPCQVKGVVPQKLWEAFWAVKDTMQAQDNNTSCRLLQQEGLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

(19) United States
(12) Patent Application Publication
(10) Pub. No.: US 2009/0004145 A1
(43) Pub. Date: Jan. 1, 2009

(54) COMPOSITIONS AND METHODS INVOLVING GENE THERAPY AND PROTEASOME MODULATION

(76) Inventor: Rajagopal Ramesh, Sugarland, TX (US)

Correspondence Address: FULBRIGHT & JAWORSKI L.L.P., 600 CONGRESS AVE., SUITE 2400, AUSTIN, TX, 78701, US

(21) Appl. No.: 11/672,896

(22) Filed: Sep. 16, 2008

Related U.S. Application Data
(60) Provisional application No. 60/771,503, filed on Feb. 8, 2006.

(51) Int. Cl. A61K 48/00; A61K 31/7088; A61P 35/00

(52) U.S. Cl. 424/93.2; 514/44

(57) ABSTRACT

The present invention concerns the methods and compositions involving a therapeutic polypeptide, such as a tumor suppressor or a nucleic acid encoding such in combination with a proteasome inhibitor, for the treatment of cancer. In certain examples, a treatment for ovarian cancer is provided.

This application claims priority to U.S. Provisional Patent Application Ser. No. 60/771,503 filed on Feb. 8, 2006, which is hereby incorporated by reference in its entirety.

The government may own rights in the present invention pursuant to grant numbers RO1-CA 102716, CA 89778, CA 88421, CA 097598, CA 16672 and PO1 CA 06294 from the National Institutes of Health

### BACKGROUND OF THE INVENTION

I. Field of the Invention

The present invention relates generally to the fields of molecular biology and oncology. More particularly, it concerns methods and compositions for treating cancer involving a tumor suppressor and one or more proteasome inhibitors.

II. Background of the Invention

Cancer is a leading cause of death in most countries, and the result of billions of dollars in healthcare expense around the world. Through great effort, significant advances have been made in treating cancer, primarily due to the development of radiation and chemotherapy-based treatments. Unfortunately, a common problem is tumor cell resistance to radiation and chemotherapeutic drugs. For example, NSCLC accounts for at least 80% of the cases of lung cancer, but patients with NSCLC are generally unresponsive to chemotherapy (Doyle, 1993). One goal of current cancer research is to find ways to improve the efficacy of these “traditional” therapeutic regimens, and the genetics of cancer cells has led to dramatic discoveries and a greater understanding of disease development.

Gene therapy has shown promise in the treatment of cancer. The goal of gene therapy in cancer therapy is the reestablishment of normal control of cellular proliferation or the elimination of cells undergoing aberrant proliferation. There are various strategies by which in vivo genetic modification can lead to therapeutic benefit. Exemplary strategies include the enhancement of immunogenicity toward the aberrant cells, the correction of a genetic defect which leads to the aberrant phenotype, and the delivery of a gene whose product is or can be made toxic to the recipient cells.

One of the best known tumor suppressor genes is p53, which plays a central role in cell cycle progression, arresting growth so that repair or apoptosis can occur in response to DNA damage. It can also initiate apoptosis if the DNA damage proves to be irreparable. Various mutant p53 alleles are known in which a single base substitution results in the synthesis of proteins that have quite different growth regulatory properties and, ultimately, lead to malignancies (Hollstein et al., 1991). In fact, the p53 gene has been found to be the most frequently mutated gene in common human cancers (Hollstein et al., 1991; Weinberg, 1991), and is particularly associated with those cancers linked to cigarette smoke (Hollstein et al., 1991; Zakut-Houri et al., 1985).

Another example of a tumor suppressor gene is melanoma differentiation-associated gene 7 (mda-7) that encodes a 24 kDa protein and is a recently described tumor suppressor gene that induces cell death and apoptosis selectively in cancer cells, while sparing normal cells (Mhashilkar et al., 2001; Mhashilkar et al., 2003; Pataer et al., 2002). Adenoviral overexpression of MDA-7 leads to tumor selective growth suppression and apoptosis induction in various tumor types including colorectal (Sarkar et al., 2002), breast (Mhashilkar et al., 2003), prostate (Mhashilkar et al., 2001), and lung carcinoma (Chada et al., 2004).

Another example of a tumor suppressor is the family tumor suppressors located in the chromosome 3p21.3 region. Human chromosome band 3p21.3 has been shown to undergo overlapping homozygous deletions in several SCLC and NSCLC lines; candidates of tumor suppressor genes have been located in this critical region in several human cancers, further defining a tumor suppressor gene region. The evidence shows that genes in this 3p21 critical region are involved in regulation of the telomerase-mediated cellular immortality pathway in lung, renal, and breast cancer cells. It has also been shown that 3p deletion occurs more frequently in the lung tumor tissues of patients who smoke. In addition, elevated sensitivity to the carcinogen benzo[a]pyrene diol epoxide at 3p21.3 has been associated with an increased risk of lung cancer, suggesting that 3p21.3 is a molecular target of carcinogens in lung cancer. Tumor suppressor genes known to be associated with this region include CACNA2D2, PL6, 101F6, NPRL2, BLU, RASSF1, FUS1, HYAL2, and HYAL1. Recent studies indicate that FUS1, 101F6 and NPRL2 are promising candidates for tumor suppressor gene or peptide therapy. (Ji et al., 2000).

Post translational protein modifications regulate protein function and stability. Among the several post translational modifications, ubiquitination signals protein degradation by the proteasome pathway (Hirano et al., 2006; Yamasaki et al., 2007). Degradation of cellular proteins through differential rate is essential in maintaining normal homeostasis of cells and preventing abnormal cellular function. Recent studies have shown several cellular proteins are degraded by the 26S proteasome (Trotman et al., 2007; Wang et al., 2007; den Besten et al., 2006). In fact one mechanism for cancer cell survival and resistance to therapy has been attributed to the rapid ubiquitination and degradation of tumor suppressor proteins.

The present invention addresses the need for additional methods and compositions related to the inhibition degradation of therapeutic tumor suppressor genes and proteins and the sensitization of cancer cells for the treatment of cancers and hyperproliferative disorders.

### SUMMARY OF THE INVENTION

Embodiments of the invention include methods and compositions related to the discovery of a relationship between the reduced expression of various components of the proteasome and resistance to chemotherapy. Embodiments of the invention include methods and compositions for the prognosis of resistance to therapy, and enhancement of gene therapies and other therapies that rely on the expression or overexpression of a polypeptide that is degraded by the proteasome pathway or acts through such polypeptide. Certain aspects of the invention include assessing the expression level of proteasome subunits or other proteins that modulate or are required for proteasome function. In other aspects of the invention proteasome inhibitors may be used to increase the expression of a therapeutic protein. Still other aspects of the invention include the administration of a therapeutic polypeptide or a nucleic acid encoding the same in combination with a proteasome inhibitor (therapeutic polypeptide/proteasome inhibitor therapy). The therapeutic peptide/proteasome inhibitor therapy may be further enhanced by administration of other cancer therapies in conjunction with the therapeutic polypeptide/proteasome inhibitor therapy. In additional aspects of the invention the regulation of proteasome subunits, e.g. beta 5 subunit, may be used to screen for compounds or treatments to downregulate the proteasome and upregulate expression of tumor suppressors and other therapeutic polypeptides. In certain embodiments the expression of a 20S proteasome subunit may be down regulated or reduced in expression to provide for a reduction in degradation of a therapeutic polypeptide, e.g., by antisense down regulation.

Exemplary embodiments of the invention include methods for enhancing the effectiveness of a gene therapy comprising administering a gene therapy in conjunction with administering a proteasome inhibitor to a subject in need of such therapy. In one aspect the degradation of a polypeptide product of the gene therapy is reduced. Gene therapy is, by definition, the insertion of genes or nucleic acid into cells for the purpose of medicinal therapy. The principle underlying gene therapy is to deliver a functional gene or nucleic acid whose RNA or protein product will produce the desired biological effect in a target cell or tissue. “Proteasome inhibitor” refers any substance which directly or indirectly decreases, reduces or inhibits the activity of the proteasome, in particular the 20S or 26S proteasome. Non-limiting examples of proteasome inhibitors for use in the present invention include peptide aldehydes (PCT publications WO 95/24914 and WO 91/13904; Iqbal et al. J. Med. Chem. 38:2276-2277 (1995)), peptide boronic acids (PCT publications WO 96/13266 and WO 91/13904), lactacystin, lactacystin analogs (Fenteany et al. Proc. Natl. Acad. Sci. USA (1994) 91:3358; PCT publication WO 96/32105), α-keto carbonyl, α-keto amide, α-keto aldehyde (glyoxals), 3,4-dichloroisocoumarin, Peptide chloromethyl/diazomethyl ketones, α,β-epoxyketones, and Peptide vinyl sulfones (3,4-DCI) (see Complex Tools for a Complex Enzyme, BOGYO and WANG). A gene therapy can be a cancer gene therapy. In certain aspects the cancer gene therapy is a tumor suppressor gene therapy, such as MDA-7 gene therapy. A cancer patient can be treated by providing an effective amount of MDA-7 and a proteasome inhibitor to the patient. Certain aspects of the invention include methods for treating ovarian, breast, or lung cancer in a patient comprising administering to the patient an effective amount of an adenovirus vector comprising a nucleic acid sequence encoding a therapeutic polypeptide, such as MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1, wherein the nucleic acid sequence is under the control of a promoter capable of being expressed in the patient; and, administering a proteasome inhibitor.

A further embodiment includes methods of assessing sensitivity of a patient to therapy comprising assessing levels of proteasome subunits in a sample of a therapeutic target. A patient or subject having a reduced expression or inhibition of the proteasome pathway will be considered a being resistant to therapy, particularly chemotherapies. The expression or activity of the proteasome may be determined by using various techniques known to one of skill in the art, such as immunodetection methods, immunoprecipitation, western blotting, immunofluorescence, and the like. Aspects of the invention include assessment of a subject diagnosed with cancer. In certain aspects the cancer is in the form of a tumor, particularly a breast or lung tumor. The proteasome subunit can be any subunit or complex of such subunits, particularly the beta 5 (accession no. BT006777 (GI:30582392, which is incorporated herein by reference)) and/or 20S subunit. The 20S subunit comprises 28 subunits in four stacked, heptameric rings. The two outer rings comprise α-subunits, and the inner rings, comprising the β-subunits form the central cavity harboring the catalytically active 5 (X, LMP7), 1 (Y, LMP2), and 2 (Z, MECL-1) subunits, which belong to the family of N-terminal nucleophile hydrolases. Proteasome 20S subunits include PSMA1 (gi:12803501; gb:AAH02577), PSMA2 (gi:29126971; gb:AAH47697), PSMA3 (gi:20810439; gb:AAH29402), PSMA4 (NP_{—}002780; GI:4506185), PSMA5 (gi:54696300; gb:AAV38522), PSMA6 (gi:12804241; gb:AAH02979), PSMA7 (gi:13325216; gb:AAH04427), PSMB1 (gi:12653473; gb:AAH00508), PSMB2 (gi:79160069; gb:AAI07902), PSMB3 (gi:15278174; gb:AAH13008), PSMB4 (gi:14249873; gb:AAH08314), PSMB5 (gi:35505516; gb:AAH57840), PSMB6 (gi:12654059; gb:AAH00835), PSMB7 (gi:30583275; gb:AAP35882), PSMB8 (gi:12654559: gb:AAH01114), PSMB9 (gi:41388830; gb:AAH65513), and PSMB10 (gi:16877952; gb:AAH17198). The methods may also include relating the levels determined with resistance to a therapy, chemotherapy, radiotherapy, immunotherapy and/or gene therapy.

In still further embodiments methods of the invention include methods for sensitizing cancer cells to therapies such as chemotherapy by providing the cells with an effective amount of a therapeutic polypeptide, such as MDA-7 and a proteasome inhibitor.

Embodiments of the invention also include pharmaceutical compositions comprising: a proteasome inhibitor or proteasome inhibitor prodrug; and an isolated nucleic acid having a sequence encoding a therapeutic polypeptide, such as an MDA-7 polypeptide. A “therapeutic polypeptide” refers to any peptide that is useful to treat a disease state or to improve the overall health of a living organism. A therapeutic polypeptide may effect such changes in a living organism when administered alone, or when administered in combinations to improve the therapeutic capacity of another substance.

Methods of the invention also include methods for treating a subject at a heightened risk of cancer or identified as having a heightened risk of cancer comprising providing an effective amount of a therapeutic polypeptide, such as MDA-7, and a proteasome inhibitor to the subject, wherein the amount of the therapeutic polypeptide and proteasome inhibitor is sufficient to reduce the risk of cancer or the recurrence of cancer in the subject.

Other methods of the invention include methods for treating or reducing cancer metastasis in a subject comprising administering to the subject an effective amount of: an adenovirus vector comprising a nucleic acid sequence encoding a therapeutic polypeptide, such as MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1, wherein the nucleic acid sequence is under the control of a promoter capable of being expressed in the subject; and an effective amount of a proteasome inhibitor.

Still other methods of the invention include methods for treating a premalignant lesion in a subject comprising providing an effective amount of a therapeutic polypeptide, such as MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1, and a proteasome inhibitor to the subject.

In the various embodiments and aspects of the invention a proteasome inhibitor can be a natural product, a peptide aldehyde, or a boronic acid inhibitor of the proteasome. A proteasome inhibitor includes, but is not limited to lactacystin, MG132, ALLN, MG115, bortezomib or combinations thereof. In certain aspects the proteasome inhibitor is MG132. A proteasome inhibitor is typically administered to the patient either intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation, by injection, by infusion, by continuous infusion, by localized perfusion bathing target cells directly, via a catheter, via a lavage or combinations thereof. A proteasome inhibitor can be administered before, after, or during administration of the gene therapy. In certain aspects the proteasome inhibitor is administering to a subject as a proteasome inhibitor prodrug, which is metabolized or otherwise converted to a proteasome inhibitor after administration to a subject.

Methods of the invention may include providing a therapeutic polypeptide to a patient by administering a composition comprising a nucleic acid having a sequence encoding a therapeutic polypeptide, such as an MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1 polypeptide, wherein the polypeptide is expressed in the patient. Typically compositions of the invention are pharmaceutically acceptable compositions. A nucleic acid of the invention can comprised in a vector, such as a viral vector. In certain aspects about 10, 10³, 10⁵, 10⁷, 10⁹, to about 10⁶, 10⁸, 10¹⁰, 10¹², 10¹³, 10¹⁴, 10¹⁵ viral particles are administered to the patient/administration. In other aspects vector is an adenovirus vector. Adenovirus vector of the invention can be formulated with protamine. Nucleic acid composition may comprise one or more lipids, and particularly DOTAP and cholesterol, or derivatives thereof. A subject may be provided with a composition comprising the proteasome inhibitor and a nucleic acid having a sequence encoding a therapeutic polypeptide. The subject can be provided with a polypeptide within 1, 2, 3, 4, 5, 6, 7, 8, 9, 10, 11, 12, 13, 14, 15, 16, 17, 18, 19, 20, 21, 22, 23, 24 or more hours or 1, 2, 3, 4, 5, 6, or more days of being provided with the proteasome inhibitor. A subject can be provided a therapeutic polypeptide or a proteasome inhibitor before during or after being provided with a proteasome inhibitor or a therapeutic polypeptide.

A cancer treated by the methods and compositions of the invention includes, but is not limited to melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In various aspects the cancer is of epithelial origins.

Methods of the invention can further comprise subjecting or providing a subject radiotherapy, chemotherapy, immunotherapy, surgical therapy, or gene therapy. A subject may be provided or subjected to additional therapy before, during or after being provided or subjected to the methods and compositions of the invention. Certain aspects include subjecting or providing a subject to a sub-lethal dose of radiotherapy or chemotherapy. In a further aspect all or part of a tumor may be resected from a subject. The methods include providing a therapeutic polypeptide, such as MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1, a proteasome inhibitor, or both a therapeutic polypeptide and a proteasome inhibitor to a subject before, during or after resection of one or more tumors from a subject. A therapeutic polypeptide, a proteasome inhibitor, or both a therapeutic polypeptide and a proteasome inhibitor can be administered a resulting tumor bed.

A subject may be provided a therapeutic polypeptide, a proteasome inhibitor, or both a therapeutic polypeptide and a proteasome inhibitor more than 1, 2, 3, 4, 5, 6, 7, 8, 9, 10 or more times.

Methods of the invention can include identification of a subject in need of inventive methods. Subjects in need of such methods include, but are not limited to subjects that have had chemotherapy, radiation therapy, immunotherapy, gene therapy or other therapies. Subjects also include those that have been determined to be resistant to a therapy or demonstrate a recurrence of cancer after therapy. In certain aspects the subject is resistant to chemotherapy, and particularly cisplatin therapy. Subjects also include those subjects that have been identified as having a condition associated with the reduction in amount or activity of proteasomes.

It will be understood that “an effective amount” means that the subject, including patients, is provided with an amount or amounts of one or more compositions that lead to a therapeutic benefit. It will be understood that the subject may given an amount of a tumor suppressor such as MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1 and an amount of a proteasome inhibitor, both in amounts that contribute to a therapeutic benefit. In embodiments, in which more than two different compounds are provided the term “effective amount” means that subject is provided with an amount that provides a therapeutic benefit as a result of the amount of the combination of substances that is provided to the subject.

“Treatment” and “treating” refer to administration or application of an agent, drug, or remedy to a subject or performance of a procedure or modality on a subject for the purpose of obtaining a therapeutic benefit of a disease or health-related condition.

A “disease” or “health-related condition” can be any pathological condition of a body part, an organ, or a system resulting from any cause, such as infection, genetic defect, and/or environmental stress. The cause may or may not be known. Examples of such conditions include, but are not limited to, premalignant states, dysplasias, cancer, and other hyperproliferative diseases. Cancer includes, but is not limited to a recurrent cancer or a cancer that is known or suspected to be resistant to conventional therapeutic regimens and standard therapies.

The term “therapeutic benefit” used throughout this application refers to anything that promotes or enhances the well-being of a subject with respect to the medical treatment of his/her condition, which includes, but is not limited to, treatment of pre-cancer, dysplasia, cancer, and other hyperproliferative diseases. A list of nonexhaustive examples of therapeutic benefit includes extension of the subject's life by any period of time, decrease or delay in the neoplastic development of the disease, decrease in hyperproliferation, reduction in tumor growth, delay of metastases or reduction in number of metastases, reduction in cancer cell or tumor cell proliferation rate, decrease or delay in progression of neoplastic development from a premalignant condition, and a decrease in pain to the subject that can be attributed to the subject's condition.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition. An amount of a pharmaceutical composition that is suitable to prevent a disease or condition is an amount that is known or suspected of blocking the onset of the disease or health-related condition.

A subject or patient can be a subject or patient who is known or suspected of being free of a particular disease or health-related condition at the time the relevant preventive agent is administered. The subject, for example, can be a subject with no known disease or health-related condition (i.e., a healthy subject). In some embodiments, the subject is a subject at risk of developing a particular disease or health-related condition. For example, the subject may have a history of cancer that has been treated in the past and is at risk of developing a recurrence of the cancer. The subject may be a subject at risk of developing a recurrent cancer because of a genetic predisposition or as a result of past chemotherapy. Alternatively, the subject may be a subject with a history of successfully treated cancer who is currently disease-free, but who is at risk of developing a second primary tumor. For example, the risk may be the result of past radiation therapy or chemotherapy that was applied as treatment of a first primary tumor. In some embodiments, the subject may be a subject with a first disease or health-related condition, who is at risk of development of a second disease or health-related condition.

“Synergistic” indicates that the therapeutic effect is greater than would have been expected based on adding the effects of each agent applied as a monotherapy.

The term “subject” includes any human, patient, or animal with, having, or is suspected of having or developing a disease or health related condition. In particular, a patient is a subject that has cancer is or will undergo treatment. In many embodiments of the invention, a subject is a mammal, specifically a human.

The term “provide” is used according to its ordinary and plain meaning: “to supply or furnish for use” (Oxford English Dictionary). The term “purified” or “isolated” means that component was previously isolated away or purified from other proteins and that the component is at least about 95% pure prior to being formulated in the composition. In certain embodiments, the purified or isolated component is about or is at least about 95, 96, 97, 98, 99, 99.1, 99.2, 99.3, 99.4, 99.5% pure or more, or any range derivable therein.

Compounds and compositions may be administered to a patient intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation, by injection, by infusion, by continuous infusion, by localized perfusion bathing target cells directly, via a catheter, or via a lavage. It is contemplated that a combination of routes of administration may be employed. For instance, a first component may be provided by one route while a second component is provided by another route. In certain aspects the first and second component may be the same or different components, e.g., that one dose is administered to a subject while another dose is administered to the subject in a different manner. In certain embodiments, it is contemplated that a compound(s) or composition(s) is directly injected into a tumor. Alternatively or additionally, a compound(s) or composition(s) is applied or administered to a residual tumor bed. Furthermore, in specific embodiments, a component is taken orally by a subject or administered intravenously to the subject.

Components and compounds of the invention can be provided to a subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more times as part of a therapy or treatment. Moreover, it is contemplated that there may be a course of therapy prescribed, and that the course may be repeated, if necessary.

In other embodiments, components or compounds of the invention are provided separately to the patient. It is contemplated that subject is provided with first agent and a second agent is provided or administered within 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7 day and/or 1, 2, 3, 4, 5, 6, 7, 8, 9, 10, 11, 12 weeks, or any range derivable therein. Consequently, a subject may take or be provided a first or second component or compound of the invention 1, 2, 3, 4, 5, 6, 7, 8, 9, 10, 11, 12, 13, 14, 15, 16, 17, 18, 19, 20 or more individual times, or any range derivable therein, within a specified time period of being provided a first or second component or compound. Alternatively, a compound may be provided systemically during or throughout treatment with a first or second component or compound of the invention.

The present invention can be used to induce apoptosis in cells. It is contemplated that this can be employed in methods and compositions for treating cancer. Cancer includes, but is not limited to melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder, including radio- and chemotherapy resistant varieties thereof. In certain embodiments, the cancer involves epithelial cancer cells. In specific embodiments, the cancer is ovarian cancer. Certain aspects of the invention are directed to chemotherapy resistant cancers and particularly ovarian cancer.

Moreover, the present invention can be used to prevent cancer or to treat pre-cancers or premalignant cells, including metaplasias, dysplasias, and hyperplasias. It may also be used to inhibit undesirable but benign cells, such as squamous metaplasia, dysplasia, benign prostate hyperplasia cells, hyperplastic lesions, and the like. The progression to cancer or to a more severe form of cancer may be halted, disrupted, or delayed by methods and compositions of the invention.

A cancer may involve an unresectable or resectable tumor. In some embodiments, the cancer appears resistant, as a monotherapy, to radiotherapy, chemotherapy, and/or immunotherapy, or to any of the agents discussed herein. Furthermore, the cancer may involve a metastasized or second tumor, though in some embodiments, it concerns only one or more primary tumors. It is further contemplated that the methods and compositions of the invention can be implemented for inhibiting metastasis of a tumor or preventing the further growth of a tumor, as well as for reducing or eliminating a tumor or cancer.

In some embodiments the patient is subjected to radiotherapy or chemotherapy after being provided compositions of the invention each at least once. In further embodiments a subject is exposed to a sub-lethal dose of therapy. The term “sub-lethal dose” refers to an amount of radiation or other therapy given to a subject in a single session that is less than a lethal amount (i.e., amount that causes cell to die) for cells of the subject exposed to the therapy. It is contemplated that a sub-lethal dose is lower than the dose currently given to a subject with similar characteristics (referring to, e.g., stage of cancer, size of tumor, prognosis, etc.) who are not first provided with a sensitization treatment. It is contemplated that sensitization treatment may precede exposure to treatment by about, at least about, or at most about 5, 10, 15, 20, 25, 30, 35, 40, 45, 50, 55, 60 minutes, and/or 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7 days, or more, or any range derivable therein.

In certain embodiments of the invention, methods also include subjecting the patient to radiotherapy and/or chemotherapy. In other embodiments, the patient is subjected to immunotherapy. In other particular embodiments, methods also involve resecting all or part of a tumor from the patient. It is contemplated that multiple tumors may be removed (whole or part). In each of these cases, methods and compositions of the invention can be provided, before, during or after the other cancer therapy.

In additional embodiments of the invention, methods include identifying a patient in need of treatment. A patient may be identified, for example, based on taking a patient history, having one or more tests done to determine that the patient has cancer or a tumor, operating on the patient or taking a biopsy.

Any lipid suitable for pharmaceutical administration is contemplated by the present invention. In certain embodiments, the composition is further defined as comprising a liposome. Any liposome suitable for pharmaceutical administration is contemplated for inclusion in the methods of the present invention. In certain embodiments, the liposome is a DOTAP:cholesterol nanoparticle. Liposomes and nanoparticles are discussed in greater detail in the specification below. The method/route of administration can be any method known to those of ordinary skill in the art, such as intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation, by injection, by infusion, by continuous infusion, by localized perfusion bathing target cells directly, via a catheter, and/or via a lavage.

In some embodiments, the patient has a history of cancer that has been successfully treated with chemotherapy, radiotherapy, chemotherapy, immunotherapy, and/or gene therapy. The patient may be administered the adenovirus vector once or more than once.

The present invention also generally pertains to methods for treating a premalignant lesion in a patient that include providing an effective amount of a tumor suppressor such as MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1 to the patient. Providing an effective amount of a tumor suppressor can be by any method known to those of ordinary skill in the art, such as by administering to the patient a composition that includes a nucleic acid having a sequence encoding the tumor suppressor polypeptide, wherein the tumor suppressor polypeptide is expressed in the patient. The composition may be a pharmaceutically acceptable composition, as discussed above. In certain embodiments, the nucleic acid is in a vector. Vectors are as discussed above, the discussion of which is incorporated into this section.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Determination of IC50 values for CDDP-Treated Ovarian Cancer Cell Lines. Human ovarian tumor (2008 and 2008/C*13R) tumor cells (2×10³) seeded in 96-well plates were treated with various doses of CDDP (0, 10, 20, 30, 60, 80, 100 μM). After 1 h of treatment, CDDP containing medium was removed and fresh medium added to the wells. Cells were incubated for 72 h following which were subjected to XTT assay to determine cell viability. Untreated cells served as controls. 2008 cells were more sensitive to CDDP with an IC50 of 30 μM while 2008/C*13 R cells were resistant to CDDP and had an IC50 value of 145 μM.

FIG. 2. Ad-mda7 plus CDDP treatment sensitizes CDDP-resistant tumor cells. CDDP-resistant ovarian tumor (2008/C*13 R) cells were plated in six-well plates and treated with PBS, CDDP (50-100 μM) Ad-mda7 (1000, 2000, 3000 vp/cell), or with varying doses of Ad-mda7 plus a fixed dose of CDDP. Treatment schedule was as follows: pretreatment with CDDP for 1 h, washed with PBS to remove any residual CDDP and then infect with Ad-mda7 for 3 h in serum free medium followed by replenishment of complete medium. Cells were observed at 72 h after treatment under bright-field microscopy. FIG. 2A, CDDP used was 100 μM; FIG. 2B, CDDP used was 50 μM. All other experimental conditions were the same in both experiments.

FIG. 3. Ad-mda7 plus CDDP treatment sensitizes CDDP-resistant tumor cells. CDDP-resistant (2008/C*13 R) and sensitive (2008) ovarian tumor cells were plated in six-well plates and treated with PBS, CDDP (25 μM) Ad-luc, Ad-mda7 (3000 vp/cell), Ad-mda7 plus CDDP or Ad-luc plus CDDP. Treatment schedule was as follows: pretreatment with CDDP for 1 h, washed with PBS to remove any residual CDDP and then infect with Ad-mda7 for 3 h in serum free medium followed by replenishment of complete medium. Cells were harvested at 72 h after treatment and number of dead cells counted by the trypan blue assay. FIG. 3A, CDDP-resistant 2008/C*13 R cells were significantly sensitized to the combined therapy of Ad-mda7 and CDDP compared to other treatment groups. FIG. 3B, percent killing in 2008 cells was similar in CDDP-, Ad-luc plus CDDP and Ad-mda7 plus CDDP-treated cells suggesting the cells were highly sensitive to CDDP.

FIG. 4. MDA-7 protein expression is increased in CDDP-Resistant Ovarian tumor cells in a time-dependent and dose-dependent manner. FIG. 4A, Ovarian tumor cells (2008 and 2008/C*13 R) seeded in six-well plates were treated with PBS, Ad-luc or Ad-mda7 (3000 vp/cell). Cells were harvested on day 1 and day 2 after treatment and analyzed for MDA-7 protein expression by western blotting. MDA-7 protein expression was observed in Ad-mda7-treated cells on both day 1 and day 2. However, MDA-7 protein expression levels was greatly increased in Ad-mda7-treated 2008/C*13 R cells compared to Ad-mda7-treated 2008 cells and in a time-dependent manner. Beta-actin was used as an internal loading control. FIG. 4B, Ovarian tumor cells (2008 and 2008/C*13 R) seeded in six-well plates were treated with different doses of Ad-mda7 (1000, 3000, 5000 or 7500 vp/cell). Untreated cells served as control. Cells were harvested on day 2 after treatment and analyzed for MDA-7 protein expression by western blotting. MDA-7 protein expression was observed in Ad-mda7-treated cells in both 2008 and 2008/C*13 R cells. Expression was observed to increase in a dose-dependent manner. However, MDA-7 protein expression levels was greatly increased in Ad-mda7-treated 2008/C*13 R cells compared to Ad-mda7-treated 2008 cells at all doses. Beta-actin was used as an internal loading control. FIG. 4C, Analyses of tissue culture supernatant from Ad-mda7-treated 2008 and 2008/C*13 R cells showed MDA-7 protein that was increased over time. However, MDA-7 protein was higher in the supernatant of Ad-mda7-treated 2008/C*13 R cells compared to Ad-mda7-treated 2008 cells.

FIG. 5. Exogenous p53 and GFP protein expression is increased in CDDP-resistant ovarian tumor cells. Ovarian tumor cells (2008 and 2008/C*13 R) seeded in six-well plates were treated with PBS, Ad-p53 (3000 vp/cell), or Nanoparticle GFP (2.5 μg DNA). Cells were harvested on day 1 and day 2 after treatment and analyzed for transgene expression by western blotting. FIG. 5A, p53 protein expression was observed in Ad-p53-treated cells on both day 1 and day 2. However, p53 protein expression levels was greatly increased/stabilized in Ad-p53-treated 2008/C*13 R cells compared to Ad-p53-treated 2008 cells. Beta-actin was used as an internal loading control. FIG. 5B, GFP protein expression was observed in Nanoparticle-GFP-treated cells on both day 1 and day 2. However, GFP protein expression levels was greatly increased in Nanoparticle-GFP-treated 2008/C*13 R cells compared to Nanoparticle-GFP-treated 2008 cells.

FIG. 6. Transduction efficiency in CDDP-resistant cells. Tumor cells (2008 and 2008/C*13 R) were treated with Ad-GFP at 10, 50, 100 and 200 vp/cell. Cells receiving no treatment served as control. At 24 h after treatment cells were harvested, washed with PBS three times, resuspended in 500 μl PBS and subjected to FACS analysis. FIG. 6A, A slight increase in number of GFP positive cells was observed in 2008/C*13 R cells compared to 2008 cells. FIG. 6B, A slight increase in GFP positive cells sorted by mean fluorescent intensity was also observed in 2008/C*13 R cells compared to 2008 cells.

FIG. 7. MDA-7 protein expression is not increased in CDDP-resistant cells due to increase adenovirus receptor expression. Tumor cells (2008 and 2008/C*13 R) were treated with Ad-mda-7 at 1000, 3000, 5000 and 7500 vp/cell. Cells receiving no treatment served as control. At 24 h after treatment cells were harvested, washed with PBS three times, resuspended in 500 μl PBS and incubated with FITC-conjugated-anti-CAR antibody, anti-alphaVbeta5 or anti-alphaVbeta3 antibody for 1 h. Cells were subsequently washed, resuspended and subjected to FACS analysis. The expression levels of CAR, alphaVbeta5 and alphaVbeta3 receptors was higher in 2008 cells compared to 2008/C*13 R cells.

FIG. 8. CDDP-resistant tumor cells are more sensitive to Ad-mda7. CDDP-resistant (2008/C*13 R) and sensitive (2008) ovarian tumor cells were plated in six-well plates and treated with PBS, Ad-luc or Ad-mda7 (3000 vp/cell). Treatment schedule followed was: treatment with Ad-mda7 for 3 h in serum free medium followed by replenishment of complete medium. Cells were harvested on day 3 and day 5 after treatment and number of dead cells counted by the trypan blue assay and analyzed for apoptotic markers by western blotting. FIG. 8A, CDDP-resistant 2008/C*13 R cells were significantly sensitized to Ad-mda7 as observed by the increase in the percent of killing compared to percent killing of 2008 cells. Increased Ad-mda7-mediated killing of 2008/C*13 R was observed at both time points. FIG. 8B, Western blotting analyses showed cleavage of caspase-3, caspase-9, and PARP on day 5 in both 2008/C*13 R and 2008 cells. However, the expression levels of these proteins were higher in 2008/C*13 R compared to 2008 cells.

FIG. 9. CDDP-resistant tumor cells have more endogenous ubiquitinated proteins. CDDP-resistant (2008/C*13 R) and sensitive (2008) ovarian tumor cells were plated in six-well plates and were untreated or treated with MG132. Cells were harvested 24 h after treatment, total cell lysate prepared and subjected to western blotting. Membrane was probed with human anti-ubiquitinated antibody. Total ubiquitinated proteins was observed to be increased in CDDP-resistant 2008/C*13 R cells compared to 2008 cells. Treatment with MG132 however greatly increased the total ubiquitinated protein levels in 2008 but not in 2008/C*13 R cells.

FIG. 10. MDA-7 protein degradation is delayed in CDDP-resistant tumor cells. CDDP-resistant (2008/C*13 R) and sensitive (2008) ovarian tumor cells were plated in six-well plates and were treated with Ad-mda7. At 24 h after treatment cells were treated with Cyclohexamide. At 3, 6, 9, 12, 24 and 48 h after cyclohexamide treatment cells were harvested, lysates prepared and subjected to MDA-7 protein detection by western blotting. MDA-7 protein expression was detectable in both 2008 and 2008/C*13 R cells. However, the half-life of MDA-7 protein was observed to be increased and more than 48 h in CDDP-resistant 2008/C*13 R cells while 10-11 h in 2008 cells indicating delayed MDA-7 protein degradation in CDDP-resistant tumor cells.

FIG. 11. Inhibition of the proteasomal activity by MG132 results in increased ubiquitinated MDA-7 protein expression in CDDP-sensitive tumor cells but not in CDDP-resistant tumor cells. FIG. 11A, CDDP-resistant (2008/C*13 R) and sensitive (2008) ovarian tumor cells were plated in six-well plates and were treated with PBS, MG132, Ad-mda7, or Ad-mda7 plus MG132. At 48 h after treatment cells were harvested, lysates and supernatants prepared and subjected to MDA-7 and total ubiquitinated protein detection by western blotting and proteasome activity assay using commercially available kit. MDA-7 protein expression was detectable in Ad-mda7 treated 2008 and 2008/C*13 R cells with increased expression observed in 2008/C*13 R compared to 2008 cells. However, the MDA-7 protein was increased in 2008 cells when treated with MG132 plus Ad-mda7. In 2008/C*13 R cells, treatment with MG132 plus Ad-mda7 did not increase MDA-7 protein expression levels. Additionally, proteasome assay revealed MG132 effectively inhibited the proteasome activity in 2008 cells (60-65% inhibition) compared to that in 2008/C*13 R cells (20% inhibition) indicating CDDP-resistant cells have reduced proteasome activity. FIG. 11B, CDDP-resistant (2008/C*13 R) and sensitive (2008) ovarian tumor cells were plated in six-well plates and were treated with PBS, MG132, Ad-p53, or Ad-p53 plus MG132. At 48 h after treatment cells were harvested, lysates and supernatants prepared and subjected to p53 protein detection by western blotting and proteasome activity using commercially available kit. P53 protein expression was detectable in Ad-p53-treated 2008 and 2008/C*13 R cells with increased expression observed in 2008/C*13 R compared to 2008 cells. However, the p53 protein was increased in 2008 cells when treated with MG132 plus Ad-p53. In 2008/C*13 R cells, treatment with MG132 plus Ad-p53 did not increase p53 protein expression levels. Additionally, proteasome assay revealed MG132 effectively inhibited the proteasome activity in 2008 cells (60-65% inhibition) compared to that in 2008/C*13 R cells (20% inhibition) indicating CDDP-resistant cells have reduced proteasome activity. Additionally, MDA-7 protein was demonstrated to be ubiquitinated.

FIG. 12. MDA-7 protein is ubiqutinated. CDDP-resistant (2008/C*13 R) and sensitive (2008) ovarian tumor cells were plated in six-well plates and were treated with PBS, or Ad-mda7. At 48 h after treatment cells were harvested, lysates prepared and subjected to immunoprecipitation using agarose-G-beads coated with A, anti-ubiquitin antibody B, anti-MDA-7 antibody. Immunoprecipiates were run on a SDS-PAGE gel electrophoresis and probed with A, anti-MDA7 antibody and B, anti-ubiquitin antibody. C, Human lung cancer cells (H1299) were treated with PBS, MG132, Ad-mda7 or Ad-mda7 plus MG132. Cells were harvested at 24 h after treatment, lysates prepared and subjected to western blotting using human anti-ubiquitin antibody and anti-MDA-7 antibody. Detection of MDA-7 protein was observed in Ad-mda7 and Ad-mda7 plus MG132-treated cells. However, increased MDA-7 protein was detected in Ad-mda7 plus MG132-treated cells indicating MDA-7 protein is ubiquitinated.

FIG. 13. Expression of 20S subunit but not 19S subunit of the 26S Proteasome is reduced in CDDP-resistant tumor cells CDDP-resistant (2008/C*13 R) and sensitive (2008) ovarian tumor cells were plated in six-well plates and were treated with PBS, or MG132. At 24 h after treatment cells were harvested, lysates prepared and subjected to western blotting. Primary antibodies used were anti-human 19S antibody and anti-human 20S antibody. No significant difference in the expression levels of 19S protein was observed in PBS or MG132-treated cells in both 2008 and 2008/C*13 R. However, expression levels of 20S protein was markedly reduced in 2008/C*13 R cells compared to 2008 cells both in PBS and MG132 treated cells indicating 20S expression is reduced in CDDP-resistant tumor cells.

FIG. 14. Expression of Beta-5 of the 20S proteasome subunit is reduced in CDDP-resistant tumor cells. CDDP-resistant (2008/C*13 R) and sensitive (2008) ovarian tumor cells were plated in six-well plates. At 24 h after plating cells were harvested, lysates prepared and subjected to western blotting. Primary antibodies used were anti-human Beta-1, Beta-2 and Beta-5 antibody. No significant difference in the expression levels of Beta-1 and Beta-2 were observed between 2008 and 2008/C*13 R cells. However, expression levels of Beta-5 protein was markedly reduced in 2008/C*13 R cells compared to 2008 cells. Beta-actin was used as internal loading control.

FIG. 15. Expression of endogenous ubiquitinated proteins is increased in CDDP-resistant tumor cells. CDDP-resistant (2008/C*13 R) and sensitive (2008) ovarian tumor cells were plated in six-well plates. At 24 h after plating cells were harvested, lysates prepared and subjected to western blotting. Primary antibodies used were anti-human p53, anti-human pJNK, antihuman-p38MAPK and anti-human p44/42MAPK antibody. Increased expression levels of p53, pJNK, p38MAPK, p44/42MAPK all of which undergo ubiquitination and proteasomal degradation were observed in 2008/C*13 R cells compared to the expression levels of these proteins in 2008 cells. Beta-actin was used as internal loading control.

FIG. 16. Expression of endogenous ubiquitinated proteins is increased in MG132-treated CDDP-sensitive tumor cells but not in CDDP-resistant tumor cells. CDDP-resistant (2008/C*13 R) and sensitive (2008) ovarian tumor cells were plated in six-well plates. At 24 h after plating cells were treated with PBS or MG132. At 24 h after treatment cells were harvested, lysates prepared and subjected to western blotting. Primary antibodies used were anti-human p53, anti-human pJNK, antihuman-p38MAPK and anti-human p44/42MAPK antibody. Increased expression levels of p53, pJNK, p38MAPK, p44/42MAPK all of which undergo ubiquitination and proteasomal degradation were observed in 2008/C*13 R cells compared to the expression levels of these proteins in 2008 cells. However, treatment with MG132 resulted in increased expression of these proteins in 2008 cells but not in 2008/C*13 R cells indicating CDDP-resistant cells do not respond to proteasomal inhibitors. Beta-actin was used as internal loading control.

FIG. 17. SiRNA-mediated inhibition of Beta-5 subunit expression of the 20S proteasome in CDDP-sensitive tumor cells. CDDP-sensitive (2008) ovarian tumor cells were plated in six-well plates. At 24 h after plating cells were treated with PBS, control SiRNA, or with four different beta-5 specific SiRNAs (100 nM). At 48 h and 72 h after treatment cells were harvested, lysates prepared and subjected to western blotting. Primary antibodies used were anti-human beta-5 antibody. A significant reduction in beta-5 protein expression was observed in cells treated with beta-5-specific siRNA compared to PBS- and control siRNA-treated cells. Beta-actin was used as internal loading control.

FIG. 18. SiRNA-mediated inhibition of Beta-5 subunit expression of the 20S proteasome in CDDP-sensitive tumor cells results in increased MDA-7 protein expression. CDDP-sensitive (2008) ovarian tumor cells were plated in six-well plates. At 24 h after plating cells were treated with PBS, control siRNA, beta-5 specific siRNAs (100 nM), Ad-mda7, Ad-mda7 plus control siRNA or Ad-mda7 plus beta-5 specific siRNA. At 48 h and 72 h after treatment cells were harvested, lysates prepared and subjected to western blotting. Primary antibodies used were anti-human beta-5 antibody and anti-human MDA-7 antibody. A significant reduction in beta-5 protein expression was observed in cells treated with beta-5-specific siRNA compared to PBS- and control SiRNA-treated cells. Correlating with inhibition of Beta-5 expression was the increase in MDA-7 protein expression. Beta-actin was used as internal loading control.

FIG. 19. SiRNA-mediated inhibition of Beta-5 subunit expression of the 20S proteasome in CDDP-sensitive tumor cells results in increased endogenous protein expression. CDDP-sensitive (2008) ovarian tumor cells were plated in six-well plates. At 24 h after plating cells were treated with PBS, control SiRNA, or beta-5 specific SiRNAs (100 nM). At 48 h after treatment cells were harvested, lysates prepared and subjected to western blotting. Primary antibodies used were anti-human beta-5 antibody, anti-human p53 antibody and anti-human pJNK antibody. A significant reduction in beta-5 protein expression was observed in cells treated with beta-5-specific siRNA compared to PBS- and control SiRNA-treated cells. Correlating with inhibition of Beta-5 expression was the increase in p53 and pJNK protein expression. Beta-actin was used as internal loading control.

FIG. 20. SiRNA-mediated inhibition of Beta-5 subunit expression of the 20S proteasome in CDDP-sensitive tumor cells abrogates proteasome activity. CDDP-sensitive (2008) ovarian tumor cells were plated in six-well plates. At 24 h after plating cells were treated with PBS, MG132, control siRNA, or beta-5 specific siRNAs (100 nM). At 48 h after treatment cells were harvested, lysates prepared and subjected to proteasome activity assay using commercially available kit. A significant inhibition of proteasome activity was observed in Beta-5-siRNA-treated cells compared to control siRNA-treated cells. However, the inhibition of proteasome activity mediated by beta-5 siRNA was less than that observed in MG132-treated cells.

FIG. 21. MDA-7 is degraded by the 26S Proteasome and not by other Proteases. CDDP-sensitive (2008) ovarian tumor cells were plated in six-well plates. At 24 h after plating cells were treated with PBS, MG132 (proteasome inhibitor), Calpain inhibitor (protease inhibitor), ammonium chloride (lysosome inhibitor), Ad-mda7, Ad-mda7 plus MG132, Ad-mda7 plus calpain inhibitor, Ad-mda7 plus ammonium chloride. At 24 h after treatment cells were harvested, lysates prepared and subjected to western blotting. Primary antibodies used were anti-human MDA-7 antibody. MDA-7 protein was detected in cells that were treated with Ad-mda7. However, a significant increase in MDA-7 protein expression was observed only in Ad-mda7 plus MG132-treated cells indicating MDA-7 undergoes proteasome-mediated degradation. Beta-actin was used as internal loading control.

### DESCRIPTION OF ILLUSTRATIVE EMBODIMENTS

Almost half of all men and more than a third of all women will be afflicted with cancer over their lifetime. Over a million people are diagnosed with it every year. While cancer death rates are generally on a decline, many people continue to die each year from some form of cancer. Lung, colon/rectal, prostate, and breast are the cancers that cause the most deaths.

Among the various cancers threatening women's health, breast cancer is the second most frequent cause of cancer-related deaths among American women. Approximately 15% of all cancer deaths reported in women area due to breast cancer and it's incidence lags only slightly behind lung cancer (Jemal et al., 2002). Furthermore, breast cancer is one of the leading causes of cancer mortality in most of the developed and developing countries throughout the world (Pisani et al., 1999). Although considerable progress has been achieved through the development of new drugs and treatment modalities, there still remains an urgent need to improve therapeutic outcomes while reducing treatment-related toxicities (Peto et al., 2000). This remains true for other cancers as well.

Cisplatin-centered chemotherapy is a key treatment for various cancers, including ovarian cancer, but resistance to chemotherapeutic agents remains a major obstacle in treatment failure. Therefore, there is an obvious need for alternative approaches, such as providing a therapeutic polypeptide by gene therapy. An example of such a therapeutic polypeptide is the melanoma differentiation associated gene 7 (mda7), as well as other tumor suppressor. MDA-7 has demonstrated the ability to work as a tumor suppressor gene in human cancer cells but not in normal cells. In exemplary studies, the growth inhibition induced by Ad-mda7 in 2008/C13*CDDP R (resistant cells) was significantly greater than that observed with 2008 CDDP S (sensitive cells) parental cell line. The expression of ectopic MDA-7 protein increased substantially in the CDDP resistant cell, whereas in the CDDP sensitive parental cell line MDA-7 protein expression decreased. Analysis of CAR receptor, αvβ3 and αvβ5 integrins in the CDDP resistant cells showed less endogenous levels and no increase in their levels when treated with Ad-mda7. On the other hand, a significant level of CAR receptor, αvβ3 and αvβ5 integrins in the CDDP sensitive cells were and no change in their levels treated with Ad-mda7 were found. The molecular mechanisms of increased adenoviral mediated MDA-7 expression in cisplatin-resistant human ovarian carcinoma cells 2008/C13*R as compared to parental cells 2008 S was investigated and discovered to correlate with a decrease in the expression and activity of the proteasome.

Another example of such a therapeutic polypeptide is p53, one of the best known tumor suppressors. This tumor suppressor is a phosphoprotein of about 390 amino acids which can be subdivided into four domains: (i) a highly charged acidic region of about 75-80 residues, (ii) a hydrophobic proline-rich domain (position 80 to 150), (iii) a central region (from 150 to about 300), and (iv) a highly basic C-terminal region. The sequence of p53 is well conserved in vertebrate species, but there have been no proteins homologous to p53 identified in lower eukaryotic organisms. Comparisons of the amino acid sequence of human, African green monkey, golden hamster, rat, chicken, mouse, rainbow trout and Xenopus laevis p53 proteins indicated five blocks of highly conserved regions, which coincide with the mutation clusters found in p53 in human cancers evolution.

p53 is located in the nucleus of cells and is very labile. Agents which damage DNA induce p53 to become very stable by a post-translational mechanism, allowing its concentration in the nucleus to increase dramatically. p53 suppresses progression through the cell cycle in response to DNA damage, thereby allowing DNA repair to occur before replicating the genome. Hence, p53 prevents the transmission of damaged genetic information from one cell generation to the next initiates apoptosis if the damage to the cell is severe. Mediators of this effect included Bax, a well-known “inducer of apoptosis.”

Embodiments of the invention include the use of tumor suppressor genes and proteasome inhibitors as a therapeutic combination against cancer and hyperproliferative disorders. In one aspect, the combination of MDA-7 and proteasome inhibitors may be used in further combination with other cancer therapies, e.g., chemotherapy, radiation therapy, immunotherapy, gene therapy and the like. In another aspect of the invention, other tumor suppressor genes in combination with proteasome inhibitors may be used to increase the cellular level of translated tumor suppressor proteins in order to inhibit or kill cancer cells or hyperproliferative cells. In particular aspects of the invention, the combination of p53 and proteasome inhibitors may be used alone or in further combination with cancer therapies e.g., chemotherapy, radiation therapy, immunotherapy, gene therapy and the like.

### I. PROTEASOME INHIBITORS

Proteasomes are multicatalytic and multi-subunit enzyme complexes which represent approximately 1% of the total cell protein and occur as the major proteolytic component in the nucleus and cytosol of all eukaryotic cells. The essential function of proteasomes is the proteolysis of misfolded or nonfunctional proteins or of regulatory proteins designed for rapid degradation. Another function of proteasomal degradation of a multiplicity of cellular or viral proteins is the generation of peptide ligands for major histocompatibility (MHC) class I molecules which are required for T-cell-mediated immune response (for a review, see Rock and Goldberg, (1999). The average human cell contains about 30,000 proteasomes, each of which contains several protein-digesting proteases. These complexes help regulate a whole host of functions including transcription, viral infection, oncogenesis, cell cycle, stress response, ribosome biogenesis, abnormal protein catabolism, neural and muscular degeneration, antigen processing, DNA repair, and cellular differentiation.

Proteins are marked for destruction by attachment of a chain of small polypeptides called ubiquitin. Ubiquitin is typically a signal for protein identification by the proteasome and processing by the proteases that make up the proteasome. Ubiquitin is attached using three enzymatic activities, E1, E2, and E3. The ATP-dependent E1 enzyme activates ubiquitin and links it to the ubiquitin-conjugating enzyme, E2. The E3 enzyme, an ubiquitin ligase, then links the ubiquitin molecule to the protein. This process repeats itself until the polypeptide is modified with a chain of ubiquitin moieties. The ubiquitin-proteasome pathway degrades 90% of all abnormal, misfolded proteins, as well as all of the short-lived, regulatory proteins in the cell. These short-lived proteins, whose half-lives are less than three hours, account for 10% to 20% of all cellular proteins. The pathway also breaks down the bulk of longer-lived proteins. The ubiquitin-proteasome pathway is responsible for degrading 80% to 90% of all the cell's proteins.

As used herein, the term “proteasome inhibitor” is intended to include inhibitors of the peptidases of the proteasome. More specifically, these inhibitors of the peptidases of the proteasome include inhibitors of the chymotrypsin-like and trypsin-like proteases, in addition to thiol and serine proteases. In addition to antibiotic inhibitors originally isolated from actinomycetes, a variety of peptide aldehydes have been synthesized, such as the inhibitors of chymotrypsin-like proteases described by Siman et al. (WO91/13904). A variety of inhibitors of the proteasome complex have been reported, e.g., Dick et al. (1991); Goldberg et al. (1992); Goldberg (1992); Orlowski (1990); Rivett et al. (1989); Rivett et al. (1989); Tanaka et al. (1992) and Myung et al. (2001). Proteasome inhibitors are also discussed in U.S. Pat. No. 5,693,617, and U.S. Patent publications 20050267037 and 2004106539 the disclosures of which are incorporated herein by reference in their entirety.

Proteasome inhibitors can be stable analogs of catalytic transition states such as Z-Gly-Gly-Leu-H, which inhibits the chymotrypsin-like activity of the proteasome (Orlowski (1990); see also Kennedy and Schultz (1979)). In addition, a variety of natural and chemical proteasome inhibitors reported in the literature, or analogs thereof, are intended to be encompassed by the present invention including peptides containing an α-diketone or an α-ketone ester, peptide chloromethyl ketone, isocoumarins, peptide sulfonyl fluorides, peptidyl boronates, peptide epoxides, and peptidyl diazomethanes. (Angelastro et al., 1990; EP Nos. 363,284, 363,284, 364,344, 393,457; PCT Pub. WO 88/10266; Ewoldt et al., 1992; Hernandez et al., 1992; Vlasak et al., 1989; Hudig et al., 1991; Odaka et al., 1991; Vijayalakshmi et al., 1991; Kam et al., 1990; Powers et al., 1989; Powers et al., 1986; Powers et al., 1990; Oweida et al., 1990; Hudig et al., 1989; Orlowski et al., 1990; Zunino et al., 1988; Kam et al., 1988; Parkes et al., 1985; Green et al., 1981; Angliker et al., 1987; Puri et al., 1989; Hanada et al., 1983; Kajiwara et al., 1987; Rao et al., 1987; Tsujinaka et al., 1988).

Peptide aldehydes and peptide α-keto esters containing a hydrophobic residue in the P1 position tested by Vinitsky et al. (1992) as potential inhibitors of the chymotrypsin-like activity of the proteasome are also intended to be encompassed by the present invention. Other tripeptides that have been described in the literature include Ac-Leu-Leu-Leu-H, Ac-Leu-Leu-Met-OR, Ac-Leu-Leu-Nle-OR, Ac-Leu-Leu-Leu-OR, Ac-Leu-Leu-Arg-H, Z-Leu-Leu-Leu-H, Z-Arg-Leu-Phe-H and Z-Arg-Ile-Phe-H, where OR, along with the carbonyl of the preceding amino acid residue, represents an ester group, and are intended to be encompassed by the present invention.

The chymotrypsin-like proteases and their inhibitors disclosed PCT publication WO 01/13904 are also intended to be encompassed by the present invention. These inhibitors have the formula R-A4-A3-A2-Y, wherein R is hydrogen, or an N-terminal blocking group; A4 is a covalent bond, an amino acid or a peptide; A3 is a covalent bond, a D-amino acid, Phe, Tyr, Val or a conservative amino acid substitution of Val; A2 is a hydrophobic amino acid or lysine or a conservative amino acid substitution thereof, or when A4 includes at least two amino acids, A2 is any amino acid; and Y is a group reactive with the active site of the protease. The peptide ketoamides, ketoacids, and ketoesters and their use in inhibiting serine proteases and cysteine proteases disclosed by PCT publication WO 92/12140 and the uses for calpain inhibitor compounds and pharmaceutical compositions containing them disclosed by PCT publication WO 92/1850 are also intended to be encompassed by the present invention.

Proteasome inhibitors include, but are not limited to: Leupeptin, Peptide Glyoxal, Peptide alpha Ketoamide, Peptide Boronic Ester, Peptide Benzamide, P′-Extended Peptide alpha Ketoamide, PSI, NLVS, Lactacystin, Eopxomicin, Eponemycin, TMC-86A, TMC-86B, TMC-89, TMC-96, YU-101, Calpain Inhibitor I, MG101, Calpain Inhibitor II, Epoxomicin, Fraction I (FrI, Hela), Fraction II (FII), clasto-Lactacystin beta-lactone, Lactacystin, ALLN, MG-115, MG-132, Antiserum to NEDD8, PA28 Activator, 20S Proteasome, Polyclonal Antibody to Proteasome 20S alpha-Type 1 Subunit, Polyclonal Antibody to Proteasome 26S Subunit S10B, Polyclonal Antibody to Proteasome 26S Subunit S2, Polyclonal Antibody to Proteasome 26S Subunit S4, Polyclonal Antibody to Proteasome 26S Subunit S5A, Polyclonal Antibody to Proteasome 26S Subunit S6, Polyclonal Antibody to Proteasome 26S Subunit S6′, Polyclonal Antibody to Proteasome 26S Subunit S7, Polyclonal antibody to Proteasome 26S Subunit S8, Polyclonal antibody to Proteasome Activator PA28 Alpha, polyclonal antibody to Proteasome Activator PA28 Gamma, Polyclonal antibody to Proteasome Activator PA700 Subunit 10B, 26S Proteasome Fraction, Proteasome Inhibitor I, Proteasome Inhibitor II, Proteasome Substrate I (Fluorogenic), Proteasome Substrate II (Fluorogenic), Proteasome Substrate III (Fluorogenic), Proteasome Substrate IV (Fluorogenic), S-100 Fraction, SUMO-1/Sentrin-1 (1-101), SUMO-1/Sentrin-1 (1-97), Antiserum to SUMO-1/Sentrin-1, Ubc10, Ubc5b, Ubc5c, Ubc6, Ubc7, Antiserum to Ubc9, Ubc9, UbCH₂/E2-14K, UbCH3/Cdc34, UbCH5a, Ubiquitin Activating Enzyme (E1), Ubiquitin Activating Enzyme (E1), Ubiquitin Aldehyde, Ubiquitin Conjugating Enzyme Fractions, Ubiquitin C-terminal Hydrolase, Ubiquitin K48R, Methylated Ubiquitin, GST-Ubiquitin, (His)6 Ubiquitin, Ubiquitin-AMC, Ubiquitin-Sepharose, boronic acid dipeptide proteasome inhibitors, and/or bortezomib (formerly PS341).

### II. NUCLEIC ACIDS

The compositions and methods of the present invention employ tumor suppressor genes and the polypeptides expressed from such genes. The term “nucleic acid” is well known in the art. A “nucleic acid” as used herein will generally refer to a molecule (i.e., a strand) of DNA, RNA (including RNAi siRNA, and ribozymes), and oligonucleotide, an oligonucleotide comprising CpG site, or a derivative or analog thereof, comprising a nucleobase. The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand. The additional strand may be partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule.

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, carboxyalkyl, amino, hydroxyl, halogen (i.e., fluoro, chloro, bromo, or iodo), thiol or alkylthiol moeity. Preferred alkyl (e.g., alkyl, carboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like.

A nucleobase may be comprised in a nucleoside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference). Any derivative or analog of a nucleoside or nucleotide that is known to those of ordinary skill in the art may be used in the methods and compositions of the present invention. A non-limiting example is a “polyether nucleic acid” and a “peptide nucleic acid.”

E. Preparation of Nucleic Acids

A nucleic acid may be made by any technique known to one of ordinary skill in the art. Examples include chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques. A non-limiting example of an enzymatically produced nucleic acid includes one produced by enzymes in amplification reactions such as PCR™ and other techniques known to those of ordinary skill in the art (see, e.g., U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

F. Nucleic Acid Complements

The present invention also encompasses a nucleic acid that is complementary to a nucleic acid encoding an amino acid sequence capable of diagnosing, treating, or preventing disease in a subject. A nucleic acid “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70% to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

### III. THERAPEUTIC NUCLEIC ACIDS

In some embodiments set forth herein, the nucleic acid is a therapeutic nucleic acid. A “therapeutic nucleic acid” is defined herein to refer to a nucleic acid which can be administered to a subject for the purpose of treating or preventing a disease. The nucleic acid is one which is known or suspected to be of benefit in the treatment of a disease or health-related condition in a subject. Diseases and health-related conditions are discussed at length elsewhere in this specification.

Therapeutic benefit may arise, for example, as a result of alteration of expression of a particular gene or genes by the nucleic acid. Alteration of expression of a particular gene or genes may be inhibition or augmentation of expression of a particular gene. In certain embodiments of the present invention, the therapeutic nucleic acid encodes one or more proteins or polypeptides that can be applied in the treatment or prevention of a disease or health-related condition in a subject. The terms “protein” and “polypeptide” are used interchangeably herein. Both terms refer to an amino acid sequence comprising two or more amino acid residues.

Any nucleic acid known to those of ordinary skill in the art that is known or suspected to be of benefit in the treatment or prevention of a disease or health-related condition is contemplated by the present invention as a therapeutic nucleic acid. The phrase “nucleic acid sequence encoding,” as set forth throughout this application, refers to a nucleic acid which directs the expression of a specific protein or peptide. The nucleic acid sequences include both the DNA strand sequence that is transcribed into RNA and the RNA sequence that is translated into protein. In some embodiments, the nucleic acid includes a therapeutic gene. The term “gene” is used to refer to a nucleic acid sequence that encodes a functional protein, polypeptide, or peptide-encoding unit.

As will be understood by those in the art, the term “therapeutic nucleic acid” includes genomic sequences, cDNA sequences, and smaller engineered gene segments that express, or may be adapted to express, proteins, polypeptides, domains, peptides, fusion proteins, and mutants. The nucleic acid may comprise a contiguous nucleic acid sequence of about 5 to about 12000 or more nucleotides, nucleosides, or base pairs.

Encompassed within the definition of “therapeutic nucleic acid” is a “biologically functional equivalent” of a therapeutic nucleic acid that has proved to be of benefit in the treatment or prevention of a disease or health-related condition. Accordingly, sequences that have about 70% to about 99% homology to a known nucleic acid are contemplated by the present invention.

A. Nucleic Acids that Encode Tumor Suppressors and Pro-Apoptotic Proteins

In some embodiments, the nucleic acid of the claimed pharmaceutical compositions include a nucleic acid sequence that encodes a protein or polypeptide that can be applied in the treatment or prevention of cancer or other hyperproliferative disease.

A “tumor suppressor” refers to a polypeptide that, when present in a cell, reduces the tumorigenicity, malignancy, or hyperproliferative phenotype of the cell. The nucleic acid sequences encoding tumor suppressor gene amino acid sequences include both the full length nucleic acid sequence of the tumor suppressor gene, as well as non-full length sequences of any length derived from the full length sequences. It being further understood that the sequence includes the degenerate codons of the native sequence or sequences which may be introduced to provide codon preference in a specific host cell.

A nucleic acid encoding a tumor suppressor generally refers to a nucleic acid sequence that reduces the tumorigenicity, malignancy, or hyperproliferative phenotype of the cell. Thus, the absence, mutation, or disruption of normal expression of a tumor suppressor gene in an otherwise healthy cell increases the likelihood of, or results in, the cell attaining a neoplastic state. Conversely, when a functional tumor suppressor gene or protein is present in a cell, its presence suppresses the tumorigenicity, malignancy or hyperproliferative phenotype of the host cell. Examples of tumor suppressors include, but are not limited to, MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide and FUS1. Other exemplary tumor suppressor genes are described in a database of tumor suppressor genes on the world wide web at cise.ufl.edu/˜yy1/HTML-TSGDB/Homepage.html. This database is herein specifically incorporated by reference into this and all other sections of the present application. Nucleic acids encoding tumor suppressor genes, as discussed above, include tumor suppressor genes, or nucleic acids derived there from (e.g., cDNAs, cRNAs, mRNAs, and subsequences thereof encoding active fragments of the respective tumor suppressor amino acid sequences), as well as vectors comprising these sequences. One of ordinary skill in the art would be familiar with tumor suppressor genes that can be applied in the present invention.

1. P53

One of the best known tumor suppressor genes is p53. p53 is central to many of the cell's anti-cancer mechanisms. It can induce growth arrest, apoptosis and cell senescence. In normal cells p53 is usually inactive, bound to the protein MDM-2, which prevents its action and promotes its degradation. Active p53 is induced after the effects of various cancer-causing agents such as UV radiation, oncogenes and some DNA-damaging drugs. DNA damage is sensed by ‘checkpoints’ in a cell's cycle, and causes proteins such as ATM, Chk1 and Chk2 to phosphorylate p53 at sites that are close to the MDM2-binding region of the protein. Oncogenes also stimulate p53 activation, mediated by the protein p14ARF. Some oncogenes can also stimulate the transcription of proteins which bind to MDM2 and inhibit its activity. Once activated p53 has many anticancer mechanisms, the best documented being its ability to bind to regions of DNA and activate the transcription of genes important in cell cycle inhibition, apoptosis, genetic stability, and inhibition of angiogenesis (Vogelstein et al, 2000). Studies have linked the p53 and pRB tumour suppressor pathways, via the protein p14ARF, raising the possibility that the pathways may regulate each other (Bates et al, 1998). Exemplary embodiments pertaining to the delivery of the p53 gene can be found in U.S. Pat. No. 6,410,010 (describing the generation of an adenoviral vector encoding p53) which is herein incorporated by reference in its entirety.

2. MDA-7

MDA-7 is a tumor suppressor that has been shown to suppress the growth of cancer cells that are p53-wild-type, p53-null and p53-mutant. MDA-7 is capable of using p53-independent mechanisms to induce the destruction of cancer cells. The compositions and methods of the present invention may employ MDA-7 polypeptides and nucleic acids encoding such polypeptides.

MDA-7 has been designated as IL-24 based on the gene and protein sequence characteristics (NCBI database accession XM_{—}001405; GeneID: 11009). The human mda-7 cDNA (SEQ ID NO:1) encodes an evolutionarily conserved protein of 206 amino acids (SEQ ID NO:2) with a predicted size of 23.8 kDa. The deduced amino acid sequence contains a hydrophobic stretch from about amino acid 26 to 45, which has characteristics of a signal sequence. A combination of structural data, homology to known cytokines, chromosomal localization, a predicted N-terminus secretion signal peptide, and evidence of its regulation of cytokine secretion, all support classification of MDA-7/IL-24 as an IL-10 family cytokine (see Chada et al., 2004 review). A 49 amino acid leader sequence identifies it as a secreted protein; recent studies confirm this and report that Ad-mda7 transduced cells release high levels of a 40 kDa form of the MDA-7 protein, which can bind to heterodimeric receptors IL-20R1/IL-20R2 and IL-22R2/IL-20R1. The intracellular form of the protein (23-30 kDa) is cleaved, and extensively modified (primarily by glycosylation) before its release into the extracellular compartment (see Chada et al., 2004 review, which is incorporated herein by reference). Effects of MDA-7 are attributable to the intracellular form of the protein and by the secreted form (bystander effect) (see U.S. patent application Ser. No. 10/791,692, which is incorporated by reference). Additional information and data regarding MDA-7 can be found in U.S. patent application Ser. Nos. 09/615,154, 10/017,472, 10/378,590, and 10/791,692, and publications by Jiang et al., 1996; and Su et al., 1998, all of which are herein incorporated by reference in their entireties.

In certain embodiments of the present invention, mda-7 is provided as a nucleic acid or polynucleotide expressing a MDA-7 polypeptide. These polynucleotides or nucleic acid molecules are isolatable and purifiable from prokaryotic and eukaryotic cells. It is contemplated that an isolated and purified mda-7 nucleic acid molecule, encoding either a secreted or full-length form of the MDA-7 polypeptide, may take the form of RNA or DNA. The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 2001; Ausubel, 1996). Alternatively, cDNAs may be advantageous because it represents coding regions of a polypeptide and eliminates introns and other regulatory regions.

It also is contemplated that a given MDA-7-encoding nucleic acid or mda-7 gene from a given cell may be represented by natural variants or strains that have slightly different nucleic acid sequences but, nonetheless, encode an MDA-7 polypeptide. Consequently, the present invention also encompasses derivatives of MDA-7 with minimal amino acid changes, but that possess the same activity.

The nucleic acid molecule encoding MDA-7 may comprise a contiguous nucleic acid sequence of the following lengths or at least the following lengths: 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 614 or more nucleotides, nucleosides, or base pairs identical or complementary to a nucleic acid encoding a tumor suppressor, for example SEQ ID NO:1 (MDA-7 encoding sequence).

In particular embodiments, the invention concerns isolated DNA segments and recombinant vectors incorporating DNA sequences that encode a tumor suppressor, for example an MDA-7 protein, polypeptide or peptide that includes within its amino acid sequence a contiguous amino acid sequence in accordance with, or essentially as set forth in, SEQ ID NO:2, corresponding to the MDA-7 designated “human MDA-7” or “MDA-7 polypeptide.”

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and may have sequences of about 70%, about 75%, about 80%, about 85%, about 90%, about 92%, about 95%, about 98%, or about 99% identical to SEQ ID NO:2, and any range derivable therein. For example, about 70% to about 80%, sequence identity to MDA-7 or fragments thereof. In certain aspects a functional equivalent will have between about 91% to about 99% sequence identity to a corresponding amino acid sequence of SEQ ID NO:2. In certain embodiments, the invention concerns isolated DNA segments and recombinant vectors that include within their sequence a nucleic acid sequence at least 70%, 75%, 80%, 85%, 90%, 95, 98, or 100% identical to that set forth in SEQ ID NO:1.

3. Tumor Suppressor Genes from Human Chromosome 3p21.3

Cytogenetic changes and allele loss on the short arm of chromosome 3 (3p) have been shown to be most frequently involved in about 90% of small cell lung cancers (SCLCs) and >50% of non-small cell lung cancers (NSCLCs) (Sekido et al., 1998; Gazdar et al., 1994; Minna, 1997; Daly et al., 1993). SCLC and NSCLC are the two treatment groups of lung tumors and are made up of four histological types. Squamous cell-, adeno-, and large cell carcinomas are in the NSCLC group. Small cell lung cancer is in the SCLC group. Approximately 75% of lung tumors are NSCLCs. Metastases occur later with NSCLC than with SCLC. SCLC is one of the most metastatic of solid tumors (Mabry et al., 1998). In addition, similar 3p changes have been seen in several other cancers in addition to lung, such as renal, breast, head and neck, pancreatic, kidney, oral, and uterine cervical cancers (Roth, 1998; Zbar et al., 1987; Gazdar et al., 1998; Sekido et al., 1998; Buchhagen et al., 1996; Gorunova et al., 1998; Hughson et al., 1998; Uzawa et al., 1998; Kersemaekers et al., 1998; Wistuba et al., 1997). Furthermore, a group of TSGs, as defined by homozygous deletions in lung cancers, have been located and isolated at 3p21.3 in a 450-kb region (Sekido et al., 1998; Minna et al., 1997; Hung et al., 1995; Sekido et al., 1996; Wistuba et al., 1999). Studies of lung cancer preneoplasia indicate that 3p21 allele loss is the earliest genetic abnormality in lung cancer detected so far, occurring in hyperplastic lesions; this shows that one or more 3p-recessive oncogenes function as “gatekeepers” in the molecular pathogenesis of many human cancers, including lung cancer, where it is likely to be involved in >50% of all cases (Sekido et al., 1998; Minna et al., 7 1997; Hung et al., 1995; Sekido et al., 1996; Wistuba et al., 1999; Kohno et al., 1999; Wistuba et al., 1999).

A group of genes has been identified in a 120-kb critical tumor homozygous deletion region (found in lung and breast cancers) of human chromosome 3p21.3. These genes have been designated CACNA2D2, PL6, 101F6, NPR1.2, BLU, RASSF1, FUS1, HYAL2 and HYAL1. Studies were conducted exploring the effect of expressing some of these genes on cell proliferation in several types of human NSCLC cells. These studies indicate that FUS1, 101F6, HYAL2 and NPRL2 as well as other genes are candidate tumor suppressor genes (Ji et al., 2000).

Restoration of wt-FUS1 function in 3p21.3-deficient human lung cancer cells by adenoviral vector- or DOTAP:cholesterol nanoparticle-mediated gene transfer inhibits the growth of these tumor cells by induction of apoptosis and alteration of cell kinetics in vitro and in vivo (Ito et al., 2004). This demonstrates the tumor suppressive activity of FUS1.

4. Other Therapeutic Nucleic Acids

a. Antisense Nucleic Acids

In some embodiments set forth herein, the nucleic acid encodes an antisense construct. Antisense methodology takes advantage of the fact that nucleic acids tend to pair with “complementary” sequences.” By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

b. RNAi

In certain embodiments of the present invention, the therapeutic nucleic acid of the pharmaceutical compositions set forth herein is an RNAi. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

One of ordinary skill in the art of RNAi understands that there are additional types of RNAi including but not limited to microRNA that may also be similarly employed in the present invention. microRNA is described in Du and Zamore, 2005, which is herein specifically incorporated by reference in its entirety.

The endoribonuclease Dicer is known to produce two types of small regulatory RNAs that regulate gene expression: small interfering RNAs (siRNAs) and microRNAs (miRNAs) (Bernstein et al., 2001; Grishok et al., 2001; Hutvagner et al., 2001; Ketting et al., 2001; Knight and Bass, 2001). In animals, siRNAs direct target mRNA cleavage (Elbashir et al., 2001), whereas miRNAs block target mRNA translation (Reinhart et al., 2000; Brennecke et al., 2003; Xu et al., 2003). Recent data suggest that both siRNAs and miRNAs incorporate into similar perhaps even identical protein complexes, and that a critical determinant of mRNA destruction versus translation regulation is the degree of sequence complementary between the small RNA and its mRNA target (Hutvagner and Zamore, 2002; Mourelatos et al., 2002; Zeng et al., 2002; Doench et al., 2003; Saxena et al., 2003). Many known miRNA sequences and their position in genomes or chromosomes can be found on the world wide web at sanger.ac.uk/Software/Rfam/mirna/help/summary.shtml.

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

U.S. Patent Publication 20050203047 reports of a method of modulating gene expression through RNA interference by incorporating a siRNA or miRNA sequence into a transfer RNA (tRNA) encoding sequence. The tRNA containing the siRNA or miRNA sequence may be incorporated into a nucleic acid expression construct so that this sequence is spliced from the expressed tRNA. The siRNA or miRNA sequence may be positioned within an intron associated with an unprocessed tRNA transcript, or may be positioned at either end of the tRNA transcript.

B. Expression Cassettes

Certain embodiments of the present invention involve therapeutic nucleic acids, wherein the nucleic acid is comprised in an “expression cassette.” Throughout this application, the term “expression cassette” is meant to include any type of genetic construct containing a nucleic acid coding for a gene product in which part or all of the nucleic acid encoding sequence is capable of being transcribed.

1. Promoters and Enhancers

In certain embodiments, the nucleic acid expressing the polypeptide is operably linked to a promoter. Non-limiting examples of promoters suitable for the present invention include a CMV IE, dectin-1, dectin-2, human CD11c, F4/80, SM22 or MHC class II promoter, however, any other promoter that is useful to drive expression of the mda-7 gene of the present invention, such as those set forth herein, is believed to be applicable to the practice of the present invention.

The term “promoter” will be used here to refer to a group of transcriptional control modules that are clustered around the initiation site for RNA polymerase II. Promoters are composed of discrete functional modules, each consisting of approximately 7-20 bp of DNA, and containing one or more recognition sites for transcriptional activator proteins. Additional promoter elements may regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another.

In some embodiments, the promoter for use in the present invention is the cytomegalovirus (CMV) immediate early (IE) promoter. Also contemplated as useful in the present invention are the dectin-1 and dectin-2 promoters. Additionally any promoter/enhancer combination (as per the Eukaryotic Promoter Data Base EPDB) could also be used to drive expression of selectable marker proteins.

Inducible Elements that may include (element/inducer/reference): MT II/Phorbol Ester (TPA) and Heavy metals/Palmiter et al. (1982), Haslinger and Karin (1985), Searle et al. (1985), Stuart et al. (1985), Imagawa et al. (1987), Karin et al. (1987), Angel et al. (1987b), McNeall et al. (1989); MMTV (mouse mammary tumor virus)/Glucocorticoids/Huang et al. (1981), Lee et al. (1981), Majors and Varmus, (1983), Lee et al. (1984), Ponta et al., (1985); β-Interferon/poly(rI)X and poly(rc)/Tavernier et al. (1983); Adenovirus 5 E2/E1a/Imperiale and Nevins (1984); Collagenase/Phorbol Ester (TPA)/Angel et al. (1987a); Stromelysin/Phorbol Ester (TPA)/Angel et al. (1987b); SV40/Phorbol Ester (TFA)/Angel et al. (1987b); Murine MX Gene/Interferon and Newcastle Disease Virus/Hug et al. (1988); GRP78 Gene/A23187/Resendez et al. (1988); α-2-Macroglobulin/IL-6/Kunz et al. (1989); Vimentin/Serum/Rittling et al. (1989); MHC Class I Gene H-2 Kb/Interferon/Blanar et al. (1989); HSP70/E1a and SV40 Large T Antigen/Taylor et al. (1989), Taylor and Kingston (1990a,b); Proliferin/Phorbol Ester-TPA/Mordacq and Linzer (1989); Tumor Necrosis Factor/PMA/Hensel et al. (1989); and Thyroid Stimulating Hormone a Gene/Thyroid Hormone/Chatterjee et al. (1989).

Other Promoter/Enhancer Elements that may be used include (Promoter and/or Enhancer/References): Immunoglobulin Heavy Chain/Banerji et al. (1983), Gillies et al. (1983), Grosschedl and Baltimore (1985), Atchinson and Perry (1986, 1987), Imler et al. (1987), Neuberger et al., (1988), Kiledjian et al. (1988); Immunoglobulin Light Chain/Queen and Baltimore (1983), Picard and Schaffner (1985); T-Cell Receptor/Luria et al. (1987), Winoto and Baltimore (1989), Redondo et al. (1990); HLA DQα and DQβ/Sullivan and Peterlin (1987); β-Interferon/Goodbourn et al. (1986), Fujita et al. (1987), Goodbourn and Maniatis (1985); Interleukin-2/Greene et al. (1989); Interleukin-2 Receptor/Greene et al. (1989), Lin et al. (1990); MHC Class II 5/, Koch et al. (1989); MHC Class II HLA-DRα/Sherman et al. (1989); β-Actin/Kawamoto et al. (1988), Ng et al. (1989); Muscle Creatine Kinase/Jaynes et al. (1988), Horlick and Benfield (1989), Johnson et al. (1989a); Prealbumin (Transthyretin)/Costa et al. (1988); Elastase I/Ornitz et al. (1987); Metallothionein/Karin et al. (1987), Culotta and Hamer (1989); Collagenase/Pinkert et al. (1987), Angel et al. (1987), Albumin Gene/Pinkert et al. (1987), Tronche et al. (1989, 1990); α-Fetoprotein/Godbout et al. (1988), Campere and Tilghman (1989); γ-Globin/Bodine and Ley (1987), Perez-Stable and Constantini (1990); β-Globin/Trudel and Constantini (1987); c-fos/Cohen et al. (1987); c-HA-ras/Triesman (1985), Deschamps et al. (1985); Insulin/Edlund et al. (1985); Neural Cell Adhesion Molecule (NCAM)/Hirsch et al. (1990); a1-Antitrypain, Latimer et al. (1990); H₂B (TH2B) Histone/Hwang et al. (1990); Mouse or Type I Collagen/Rippe et al. (1989); Glucose-Regulated Proteins (GRP94 and GRP78)/Chang et al. (1989); Rat Growth Hormone/Larsen et al. (1986); Human Serum Amyloid A (SAA)/Edbrooke et al. (1989); Troponin I (TN I)/Yutzey et al. (1989); Platelet-Derived Growth Factor/Pech et al. (1989); Duchenne Muscular Dystrophy/Klamut et al. (1990); SV40/Banerji et al. (1981), Moreau et al. (1981), Sleigh and Lockett (1985), Firak and Subramanian (1986), Herr and Clarke (1986), Imbra and Karin (1986), Kadesch and Berg (1986), Wang and Calame (1986), Ondek et al. (1987), Kuhl et al. (1987), Schaffner et al. (1988); Polyoma/Swartzendruber and Lehman (1975), Vasseur et al. (1980), Katinka et al. (1980, 1981), Tyndell et al. (1981), Dandolo et al. (1983), Hen et al. (1986); Campbell and Villarreal, (1988); Retroviruses/Kriegler and Botchan (1983), Kriegler et al., (1984a,b), Bosze et al. (1986), Miksicek et al. (1986), Celander and Haseltine (1987), Thiesen et al. (1988), Celander et al. (1988), Choi et al. (1996), Reisman and Rotter (1989); Papilloma Virus/Campo et al. (1983), Lusky et al. (1983), Spandidos and Wilkie (1983), Spalholz et al. (1985), Lusky and Botchan (1986), Cripe et al. (1987), Gloss et al. (1987), Hirochika et al. (1987), Stephens and Hentschel (1987); Hepatitis B Virus/Bulla and Siddiqui (1988), Jameel and Siddiqui (1986), Shaul and Ben-Levy (1987), Spandau and Lee (1988); Human Immunodeficiency Virus/Muesing et al. (1987), Hauber and Cullan (1988), Jakobovits et al. (1988), Feng and Holland (1988), Takebe et al. (1988), Berkhout et al. (1989), Laspia et al. (1989), Sharp and Marciniak (1989), Braddock et al. (1989); Cytomegalovirus/Weber et al. (1984), Boshart et al. (1985), Foecking and Hofstetter (1986); and Gibbon Ape Leukemia Virus/Holbrook et al. (1987), Quinn et al. (1989).

Polyadenylation signals include, but are not limited to human growth hormone (hGH) gene, the bovine growth hormone (BGH) gene, or SV40 polyadenylation signal.

2. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

3. IRES

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819). One of ordinary skill in the art would be familiar with the application of IRES in gene therapy.

### IV. PROTEINS, PEPTIDES AND POLYPEPTIDES

The present invention is directed to methods and compositions of tumor suppressor polypeptides provided as a polypeptide or a nucleic acid encoding such. The terms “protein” and “polypeptide” are used interchangeably herein.

Certain embodiments of the invention encompass the use of a purified protein composition comprising a tumor suppressor, for example an MDA-7 protein and/or a truncated version of MDA-7 lacking its endogenous signal sequence or an MDA-7 polypeptide with a heterologous signal sequence. In all examples using MDA-7 any other tumor suppressor described herein may be substituted and vice versa. Truncated molecules of MDA-7 include, for example, molecules beginning approximately at MDA-7 amino acid residues 46 to 49 and further N-terminal truncations. Specifically contemplated are molecules start at residue 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, and 182, and terminate at residue 206. In additional embodiments, residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, and 48 are included with other contiguous residues of MDA-7, as shown in SEQ ID NO:2, or any other tumor suppressor described herein.

As will be understood by those of skill in the art, modification and changes may be made in the structure of a tumor suppressor, for example an MDA-7 polypeptide or peptide, and still produce a molecule having like or otherwise desirable characteristics. For example, certain amino acids may be substituted for other amino acids in a protein structure without appreciable loss of interactive binding capacity with structures such as, for example, antigen-binding regions of antibodies. Since it is the interactive capacity and nature of a protein that defines that protein's biological functional activity, certain amino acid sequence substitutions can be made in a protein sequence (or, of course, its underlying DNA coding sequence) and nevertheless obtain a protein with similar tumor suppressive, apoptosis-inducing, antigenic, or cytokine properties. It is thus contemplated by the inventors that various changes may be made in the sequence of MDA-7 polypeptides or peptides (or underlying DNA) without appreciable loss of their biological utility or activity.

In terms of functional equivalents, the skilled artisan also understands that inherent in the definition of a biologically-functional equivalent protein or peptide, is the concept of a limit to the number of changes that may be made within a defined portion of a molecule that still result in a molecule with an acceptable level of equivalent biological activity. It is also well understood that where certain residues are shown to be particularly important to the biological or structural properties of a protein or peptide such residues may not generally be exchanged.

Amino acid substitutions are generally based on the relative similarity of the amino acid side-chain substituents, for example, their hydrophobicity, hydrophilicity, charge, size, and the like. An analysis of the size, shape, and type of the amino acid side-chain substituents reveals that arginine, lysine, and histidine are all positively charged residues; that alanine, glycine, and serine are all a similar size; and that phenylalanine, tryptophan, and tyrosine all have a generally similar shape. Therefore, based upon these considerations, the following subsets are defined herein as biologically functional equivalents: arginine, lysine, and histidine; alanine, glycine, and serine; and phenylalanine, tryptophan, and tyrosine.

A. In Vitro Protein Production

General procedures for in vitro protein production are well known to those skilled in the art of protein production. Cell culture techniques are well documented and are disclosed herein by reference (Freshney, 1992). One embodiment of the foregoing involves the use of nucleic acid transfer to immortalize cells for the production and/or presentation of proteins. The nucleic acid encoding a protein of interest may be transferred as described above into appropriate host cells followed by culture of cells under the appropriate conditions. The generation of recombinant expression vectors, and the elements included therein, are discussed above. Alternatively, the protein to be produced may be an endogenous protein normally synthesized by the cell in question. Examples of mammalian host cell lines include Vero and HeLa cells, other B- and T-cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

B. Methods of MDA-7 Purification

The present invention employs, in some embodiments, purified MDA-7. The following methods and similar methods known to one of ordinary skill in the art can be used in purification and isolation of MDA-7. Such methods are disclosed in U.S. patent application Ser. No. 10/791,692, which is hereby incorporated by reference, and briefly described below. MDA-7 may be purified using methodology recognized in the art and may include various chromatographic techniques including, but not limited to affinity, anion exchange, size exclusion, and/or lectin chromatography steps, as well as various combinations of adsorption, affinity, partition, ion exchange and molecular sieve, and many specialized techniques for using them including column, paper, thin layer and gas chromatography (Freifelder, 1982).

Certain aspects of the claimed invention involve use of immunological reagents. In certain embodiments of the claimed invention, immunological reagents are used in the purification of preparations of MDA-7. Such antibodies can be readily created and/or are readily available (see U.S. patent application Ser. No. 10/791,692). Monoclonal antibodies (MAbs) may be readily prepared through use of well-known techniques, such as those exemplified in U.S. Pat. No. 4,196,265, incorporated herein by reference.

### V. VECTORS

A. Viral Vectors

A viral vector is a virus that can transfer genetic material from one location to another, such as from the point of application to a target cell of interest. One of ordinary skill in the art would be familiar with the various types of viruses that are available for use as vectors for gene delivery to a target cell of interest. Each of these is contemplated as a vector in the present invention. Exemplary vectors are discussed below.

1. Adenoviral Vectors

The embodiments of the present invention may involve expression constructs of the therapeutic nucleic acids comprised in adenoviral vectors for delivery of the nucleic acid. Although adenovirus vectors are known to have a low capacity for integration into genomic DNA, this feature is counterbalanced by the high efficiency of gene transfer afforded by these vectors.

Adenoviruses are currently the most commonly used vector for gene transfer in clinical settings. Among the advantages of these viruses is that they are efficient at gene delivery to both nondividing an dividing cells and can be produced in large quantities. In many of the clinical trials for cancer, local intratumor injections have been used to introduce the vectors into sites of disease because current vectors do not have a mechanism for preferential delivery to tumor. In vivo experiments have demonstrated that administration of adenovirus vectors systemically resulted in expression in the oral mucosa (Clayman et al., 1995). Topical application of Ad-βgal and Ad-p53-FLAG on organotypic raft cultures has demonstrated effective gene transduction and deep cell layer penetration through multiple cell layers (Eicher et al., 1996). Therefore, gene transfer strategy using the adenoviral vector is potentially feasible in patients at risk for lesions and malignancies involving genetic alterations in p53.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.), is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 109-1011 plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

2. Retroviral Vectors

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

3. Adeno-Associated Virus Vectors

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. Nos. 5,139,941 and 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte and Carter, 1995).

4. Herpes Virus Vectors

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975). The expression of a genes, the first set of genes to be expressed after infection, is enhanced by the virion protein number 16, or a transinducing factor (Post et al., 1981; Batterson and Roizman, 1983). The expression of β genes requires functional α gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

5. Pox Virus Vectors

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

6. Oncolytic Viral Vectors

Oncolytic viruses are also contemplated as vectors in the present invention. Oncolytic viruses are defined herein to generally refer to viruses that kill tumor or cancer cells more often than they kill normal cells. Exemplary oncolytic viruses include adenoviruses which overexpress ADP. These viruses are discussed in detail in U.S. Patent Publication numbers 20040213764 and 20020028785, and U.S. patent application Ser. No. 09/351,778, each of which is specifically incorporated by reference in its entirety into this section of the application and all other sections of the application. Exemplary oncolytic viruses are discussed elsewhere in this specification. One of ordinary skill in the art would be familiar with other oncolytic viruses that can be applied in the pharmaceutical compositions and methods of the present invention.

7. Additional Viral Vectors

Other viral vectors that may be employed as vectors in the present invention include those viral vectors that can be applied in vaccines, or in dual vaccine and immunotherapy applications. Viral vectors, and techniques for vaccination and immunotherapy using viral vectors, are described in greater detail in PCT application WO0333029, WO0208436, WO0231168, and WO0285287, each of which is specifically incorporated by reference in its entirely for this section of the application and all other sections of this application. Additional vectors that can be applied in the techniques for vaccination and dual immunotherapy/vaccination include those oncolytic viruses set forth above.

Other viral vectors also include baculovirus vectors, parvovirus vectors, picornavirus vectors, alphavirus vectors, semiliki forest virus vectors, Sindbis virus vectors, lentivirus vectors, and retroviral vectors. Vectors derived from viruses such as poxvirus may be employed. A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been suggested that VEE may be an extremely useful vector for immunizations (Caley et al, 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Other viral vectors for application in the compositions and methods of the present invention include those vectors set forth in Tang et al., 2004, which is herein specifically incorporated by reference in its entirety for this section of the application and all other sections of the application.

B. Non-Viral Vectors

In certain embodiments of the present invention, the nucleic acid encoding an amino acid sequence may further comprise a delivery agent. A delivery agent is defined herein to refer to any agent or substance, other than a viral vector, that facilitates the delivery of the nucleic acid to a target cell of interest. Exemplary delivery agents include lipids and lipid formulations, including liposomes. In certain embodiments, the lipid is comprised in nanoparticles. A nanoparticle is herein defined as a submicron particle. For example, the nanoparticle may have a diameter of from about 1 to about 500 nanometers. The particle can be composed of any material or compound. In the context of the present invention, for example, a “nanoparticle” may include certain liposomes that have a diameter of from about 1 to about 500 nanometers.

1. Lipid Vectors

One of ordinary skill in the art would be familiar with use of liposomes or lipid formulation to entrap nucleic acid sequences. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use subcutaneous, intradermal, intratumoral, or intracranial injection to avoid the toxicity and stability problems associated with cationic lipids in the circulation. The interaction of liposomes and plasma proteins is responsible for the disparity between the efficiency of in vitro (Felgner et al., 1987) and in vivo gene transfer (Zhu et al., 1993; Solodin et al., 1995; Liu et al., 1995; Thierry et al., 1995; Tsukamoto et al., 1995; Aksentijevich et al., 1996).

Recent advances in liposome formulations have improved the efficiency of gene transfer in vivo (WO 98/07408). A novel liposomal formulation composed of an equimolar ratio of 1,2-bis(oleoyloxy)-3-(trimethyl ammonio)propane (DOTAP) and cholesterol significantly enhances systemic in vivo gene transfer, approximately 150 fold. The DOTAP:cholesterol lipid formulation is said to form a unique structure termed a “sandwich liposome.” This formulation is reported to “sandwich” DNA between an invaginated bi-layer or ‘vase’ structure. Beneficial characteristics of these liposomes include a positive p, colloidal stabilization by cholesterol, two dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

The liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

In addition, one of ordinary skill in the art is aware of other nanoparticle formulations suitable for gene delivery. Examples include those nanoparticle formulations described by Bianco (2004) and Doerr (2005) each of which is herein specifically incorporated by reference in its entirety.

2. Protamine Vectors

Protamines are small highly basic nucleoproteins associated with DNA. Protamine may also be used to form a complex with an expression construct. Such complexes may then be formulated with lipid compositions described herein for administration to a cell. Their use in the delivery of nucleic acids is described in U.S. Pat. No. 5,187,260, which is incorporated by reference. U.S. patent application Ser. No. 10/391,068 (filed Mar. 24, 2003), which pertains to methods and compositions for increasing transduction efficiency of a viral vector by complexing the viral vector with a protamine molecule, is specifically incorporated by reference herein.

### VI. THERAPIES

The primary modality for the treatment of cancer using gene therapy is the induction of apoptosis. This can be accomplished by either sensitizing the cancer cells to other agents or inducing apoptosis directly by stimulating intracellular pathways. Other cancer therapies take advantage of the need for the tumor to induce angiogenesis to supply the growing tumor with necessary nutrients. Endostatin and angiostatin are examples of two such therapies (WO 00/05356 and WO 00/26368).

The treatment of a wide variety of cancerous states is within the scope of the invention, e.g., melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, lymphoma, brain, colon or bladder. Other aspects of the invention include angiogenesis-related diseases such as rheumatoid arthritis, inflammatory bowel disease, osteoarthritis, leiomyomas, ademonas, lipomas, hemangiomas, fibromas, vascular occlusion, restenosis, atherosclerosis, pre-neoplastic lesions, carcinoma in situ, oral hairy leukoplakia or psoriasis may be the subject of treatment. The term “cancer” includes tumors that may or may not be resectable. Moreover, the cancer may involve metastatic tumor(s) or a tumor possibly capable of metastasis.

Cancer cells that may be treated by methods and compositions of the invention also include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### VII. PHARMACEUTICAL FORMULATIONS AND DELIVERY

Methods of the present invention include the delivery of an effective amount of a tumor suppressor protein or gene or an expression construct encoding the same, and a proteasome inhibitor. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

A. Administration

In certain embodiments, it is desired to kill cells, inhibit cell growth, inhibit metastasis, decrease tumor or tissue size, and/or reverse or reduce the malignant phenotype of tumor cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Direct injection, intratumoral injection, or injection into the tumor vasculature is specifically contemplated for discrete, solid, accessible tumors, or other accessible target areas. Local, regional, or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as a single dose comprise about 0.1 to about 0.5 ml volumes. Compositions of the invention may be administered in multiple injections to a tumor or a targeted site. In certain aspects, injections may be spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a tumor suppressor gene or protein a proteasome inhibitor or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Continuous administration also may be applied where appropriate, for example, where a tumor or other undesired affected area is excised and the tumor bed or targeted site is treated to eliminate residual, microscopic disease. Delivery via syringe or catheterization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on tumor type, tumor location, immune condition, target site, disease progression, and health and age of the patient. Certain tumor types will require more aggressive treatment. The clinician will be best suited to make such decisions based on the known efficacy and toxicity (if any) of the therapeutic formulations.

In certain embodiments, the tumor or affected area being treated may not, at least initially, be resectable. Treatments with compositions of the invention may increase the resectability of the tumor due to shrinkage at the margins or by elimination of certain particularly invasive portions. Following treatments, resection may be possible. Additional treatments subsequent to resection may serve to eliminate microscopic residual disease at the tumor or targeted site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. With respect to a viral component of the present invention, a unit dose may conveniently be described in terms of plaque forming units (pfu) or viral particles for a viral construct. Unit doses range from 10³, 10⁴, 10⁵, 10⁶, 10⁷, 10⁸, 10⁹, 10¹⁰, 10¹¹, 10¹², 10¹³ pfu or viral particles (vp) and higher. Alternatively, the amount specified may be the amount administered as the average daily, average weekly, or average monthly dose.

Protein components of the inventive compositions may be administered to a patient in doses of about or of at least about 0.01, 0.05, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 15, 20, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000 or more ng/ml, or any range derivable therein. Alternatively, any amount specified herein may be the amount administered as the average daily, average weekly, or average monthly dose.

Proteasome inhibitors can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

B. Injectable Compositions and Formulations

In some embodiments, the method for the delivery an expression construct encoding a tumor suppressor gene a proteasome inhibitor or combinations thereof is via systemic administration. However, the pharmaceutical compositions disclosed herein may also be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acid constructs may be delivered by syringe or any other method used for injection of a solution, as long as the expression construct can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising a tumor suppressor protein or a nucleic acid encoding the same is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various other ingredients enumerated above followed by filtered sterilization, as required. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, methods of preparation include vacuum-drying and freeze-drying techniques which yield a powder of the active ingredient plus any additional desired ingredient from a previously sterile-filtered solution.

The compositions disclosed herein may be formulated in a neutral or salt form. Pharmaceutically acceptable salts, include the acid addition salts (formed with the free amino groups of a protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

Compounds and agents may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The protein, nucleic acid, or proteasome inhibitor(s) compounds are administered in a manner compatible with the dosage formulation, and in such amount as will be therapeutically effective. The quantity to be administered depends on the subject to be treated, including, e.g., the aggressiveness of the cancer, the size of any tumor(s), the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

C. Combination Treatments

In certain embodiments, the compositions and methods of the present invention involve an tumor suppressor polypeptide, or expression construct encoding a tumor suppressor gene, in combination with a proteasome inhibitor. The combination of these compositions may enhance the effect of the tumor suppressor, or increase the therapeutic effect of another anti-cancer or anti-hyperproliferative being employed. These compositions would be provided in a combined amount effective to achieve the desired effect, such as the killing of a cancer cell and/or the inhibition of cellular hyperproliferation. This process may involve contacting the cells with the expression construct and the proteasomes inhibitor(s) at the same time. This may be achieved by contacting the cell with one or more compositions or pharmacological formulation that includes or more of the agents, or by contacting the cell with two or more distinct compositions or formulations, wherein one composition provides 1) MDA-7 (either as a protein or nucleic acid encoding the protein); and/or 2) a proteasome inhibitor(s). A third composition may be administered that includes a chemotherapy, radiotherapy, surgical therapy, immunotherapy or gene therapy.

In embodiments of the present invention, it is contemplated that a tumor suppressor nucleic acid or protein therapy is used in conjunction with a proteasome inhibitor (referred to as “TSG/proteasome inhibitor therapy”), in addition to a second anti-cancer agent or therapy. A TSG/proteasome inhibitor therapy may precede or follow the other anti-cancer treatment by intervals ranging from minutes to weeks. In embodiments where the tumor suppressor gene or protein therapy is provided to the patient separately from the proteasome inhibitor, one would generally ensure that a significant period of time did not expire between the time of each delivery, such that the two compounds would still be able to exert an advantageously combined effect on the patient; alternatively, in embodiments where the TSG/proteasome inhibitor therapy is provided to the patient separately from the second anti-cancer therapy, one would generally ensure that a significant period of time did not expire between the time of each therapy, such that the two therapies would still be able to exert an advantageously combined effect on the patient. In such instances, it is contemplated that one may provide a patient with the TSG/proteasome inhibitor therapy and the second anti-cancer therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example tumor suppressor gene therapy, tumor suppressor protein therapy or TSG/proteasome inhibitor therapy is “A” and the proteasome inhibitor or other therapy is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second anti-cancer therapy, such as chemotherapy, radiotherapy, immunotherapy, surgical therapy or other gene therapy, is employed in combination with the TSG/proteasome inhibitor therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

(1) Busulfan

Busulfan (also known as myleran) is a bifunctional alkylating agent. Busulfan is known chemically as 1,4-butanediol dimethanesulfonate. Busulfan is not a structural analog of the nitrogen mustards. Busulfan is available in tablet form for oral administration. Each scored tablet contains 2 mg busulfan and the inactive ingredients magnesium stearate and sodium chloride. Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

(2) Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino]benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remington's Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation. Thus, it can be used in combination with troglitazone in the treatment of cancer.

(3) Platinum-Containing Compounds

Platinum cytotoxics play an important role globally in the management of solid tumors. Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 10 mg/m². Of course, all of these dosages are exemplary, and any dosage in-between these points is also expected to be of use in the invention. Cisplatin is not absorbed orally and must therefore be delivered via injection intravenously, subcutaneously, intratumorally or intraperitoneally.

Carboplatin, another platinum compound, is associated with neurotoxicity, but has become the leading product in the U.S. due largely to the ease with which toxicity profiles can be managed. Oxaliplatin (Europe) and nedaplatin (in Japan) have also been introduced. Platinum compounds can be used effectively in combination with 5-FU. CTI is testing two additional platinum compounds—BBR 3464 and BBR 3610—to identify appropriate clinical formulations.

(4) Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

(5) Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine. Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa1 of ˜2.1. Melphalan is available in tablet form for oral administration and has been used to treat multiple myeloma. Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject

b. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

c. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

(1) Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

(2) Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr or the body surface less than 0.5 m², in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are given as an example, and any dosage in-between these points is also expected to be of use in the invention.

(3) Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg, 20 mg, or 10 mg I.V., the maximal serum concentrations were 2.4 mg/ml, 1.7 mg/ml, and 0.52 mg/ml, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

(4) Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors that fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

(5) Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin. Bleomycin may be given by the intramuscular, intravenous, or subcutaneous routes. It is freely soluble in water.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

d. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

(1) Etoposide (VP16)

VP16 is also known as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

(2) Taxanes

Taxanes are a group of drugs that includes paclitaxel (Taxol) and docetaxel (Taxotere). Taxanes prevent growth of cancer cells by inhibiting the breakdown of microtubules, which normally occurs once a cell stops dividing. Thus, treated cells become so clogged with microtubules that they cannot grow and divide.

Paclitaxel (Taxol) is isolated from the bark of the ash tree, Taxus brevifolia. It binds to tubulin (at a site distinct from that used by the vinca alkaloids) and promotes the assembly of microtubules. It has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention. Docetaxel, a compound that is similar to paclitaxel, and is also used to treat cancer. Docetaxel comes from the needles of the yew tree. The FDA has approved docetaxel to treat advanced breast, lung, and ovarian cancer.

(3) Vinblastine

Vinblastine is another example of a plant alkyloid that can be used in combination with troglitazone for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m2, 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

(4) Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can also be administered. Alternatively 0.02 mg/m², 0.05 mg/m2, 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

(5) Camptothecin

Camptothecin is an alkaloid derived from the Chinese tree Camptotheca acuminata Decne. Camptothecin and its derivatives are unique in their ability to inhibit DNA Topoisomerase by stabilizing a covalent reaction intermediate, termed “the cleavable complex,” which ultimately causes tumor cell death. It is widely believed that camptothecin analogs exhibited remarkable anti-tumour and anti-leukaemia activity. Application of camptothecin in clinic is limited due to serious side effects and poor water-solubility. At present, some camptothecin analogs (topotecan; irinotecan), either synthetic or semi-synthetic, have been applied to cancer therapy and have shown satisfactory clinical effects. The molecular formula for camptothecin is C₂₀H₁₆N₂O₄, with a molecular weight of 348.36. It is provided as a yellow powder, and may be solubilized to a clear yellow solution at 50 mg/ml in DMSO 1N sodium hydroxide. It is stable for at least two years if stored at 2-8° C. in a dry, airtight, light-resistant environment.

e. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

(1) Carmustine

Carmustine (sterile carmustine) is one of the nitrosoureas used in the treatment of certain neoplastic diseases. It is 1,3bis-(2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m², 40 mg/m², 50 mg/m², 60 mg/m², 70 mg/m², 80 mg/m², 90 mg/m² or 100 mg/m². The skilled artisan is directed to “Remington's Pharmaceutical Sciences,” 15th Edition, chapter 61. Some variation in dosage will necessarily occur depending on the condition of the subject being treated. The person responsible for administration will, in any event, determine the appropriate dose for the individual subject.

(2) Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per ml) and in absolute alcohol (70 mg per ml). Lomustine is relatively insoluble in water (<0.05 mg per ml). It is relatively unionized at a physiological pH. Inactive ingredients in lomustine capsules are magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours. The serum half-life of the metabolites ranges from 16 hrs to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m², 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques. Stereotactic radiotherapy is used to treat brain tumors. This technique directs the radiotherapy from many different angles so that the dose going to the tumour is very high and the dose affecting surrounding healthy tissue is very low. Before treatment, several scans are analyzed by computers to ensure that the radiotherapy is precisely targeted, and the patient's head is held still in a specially made frame while receiving radiotherapy. Several doses are given.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment you will have a specially made metal frame attached to your head. Then several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with MDA-7/proteasome inhibitor therapy. The general approach for combined therapy is discussed herein. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Table 1 is a non-limiting list of several known anti-cancer immunotherapeutic agents and their targets. It is contemplated that one or more anti-cancer therapies may be employed with the tumor suppressor therapies described herein.

TABLE 1

| Generic Name | Target |
|---|---|
| cetuximab | EGFR |
| panitumumab | EGFR |
| trastuzumab | erbB2 receptor |
| bevacizumab | VEGF |
| alemtuzumab | CD52 |
| gemtuzumab ozogamicin | CD33 |
| rituximab | CD20 |
| tositumomab | CD20 |
| matuzumab | EGFR |
| ibritumomab tiuxetan | CD20 |
| tositumomab | CD20 |
| HuPAM4 | MUC1 |
| MORAb-009 | Mesothelin |
| G250 | carbonic anhydrase IX |
| mAb 8H9 | 8H9 antigen |
| M195 | CD33 |
| ipilimumab | CTLA4 |
| HuLuc63 | CS1 |
| alemtuzumab | CD53 |
| epratuzumab | CD22 |
| BC8 | CD45 |
| HuJ591 | Prostate specific membrane antigen |
| hA20 | CD20 |
| lexatumumab | TRAIL receptor-2 |
| pertuzumab | HER-2 receptor |
| Mik-beta-1 | IL-2R |
| RAV12 | RAAG12 |
| SGN-30 | CD30 |
| AME-133v | CD20 |
| HeFi-1 | CD30 |
| BMS-663513 | CD137 |
| Volociximab | anti-α5β1 integrin |
| GC1008 | TGFβ |
| HCD122 | CD40 |
| Siplizumab | CD2 |
| MORAb-003 | Folate receptor alpha |
| CNTO 328 | IL-6 |
| MDX-060 | CD30 |
| Ofatumumab | CD20 |
| SGN-33 | CD33 |

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient (Irie and Morton, 1986; Irie et al., 1989; Bajorin et al., 1988).

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Gene Therapy

In yet another embodiment, a combination treatment involves gene therapy in which a therapeutic polynucleotide is administered before, after, or at the same time as a therapeutic polypeptide, such as a tumor suppressor gene or nucleic acid encoding the therapeutic polypeptide. In other embodiments a gene therapy may be used in combination with a proteasome inhibitor. Delivery of a tumor suppressor polypeptide or encoding nucleic acid in conjunction with a vector encoding one of the following gene products, or the delivery of one of the following gene therapies combined with administration of a proteasome inhibitor may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Recent studies have also shown MDA-7 protein undergoes post-translational modification (PTPM) such as phosphorylation and glycosylation, a finding not reported for other IL-10-related cytokines (Murata, 2006; Brignole et al., 2006). Since mda-7 functions and undergoes modification like other known tumor suppressor proteins we were interested in determining whether the protein undergoes additional modifications especially ubiquitination. Additionally, there are no reports demonstrating MDA-7 protein ubiquitination or its degradation.

In the present study we investigated whether mda7 protein is ubiquitinated and degraded by the 26S proteasome and that inhibition of ubiquitin-mediated MDA-7 protein degradation enhances tumor cell killing. In addition, we investigated the role of proteasome inhibition and administration of the mda7 protein in the sensitization of cisplatin resistant cell lines.

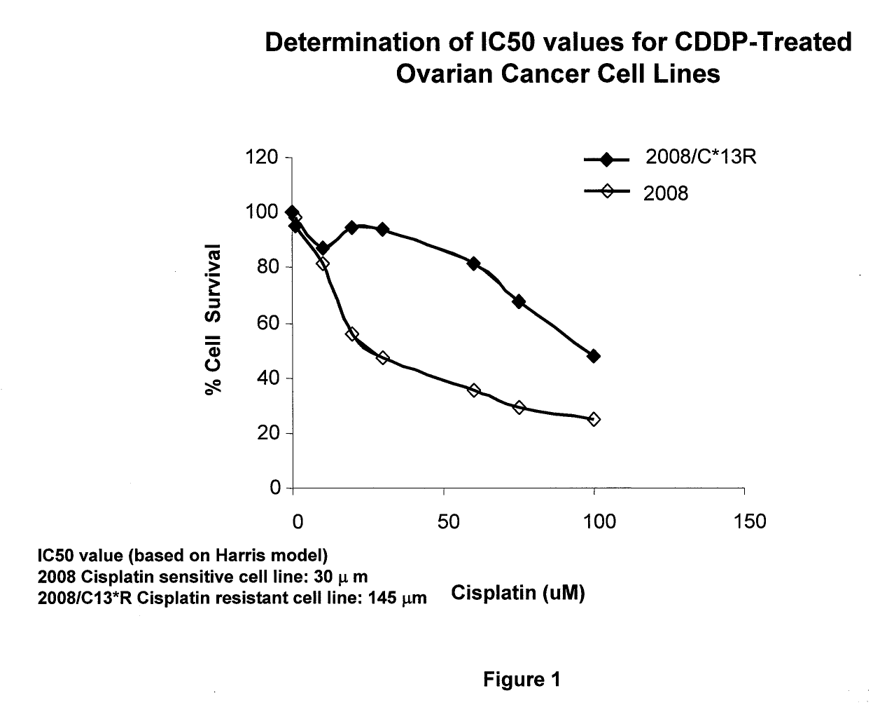

Determination of IC50 values for CDDP-Treated Ovarian Cancer Cell Lines. Human ovarian tumor (2008 and 2008/C*13R) tumor cells (2×10³) seeded in 96-well plates were treated with various doses of CDDP (0, 10, 20, 30, 60, 80, 100 μM). After 1 h of treatment, CDDP containing medium was removed and fresh medium added to the wells. Cells were incubated for 72 h following which were subjected to XTT assay to determine cell viability. Untreated cells served as controls. 2008 cells were more sensitive to CDDP with an IC50 of 30 μM while 2008/C*13 R cells were resistant to CDDP and had an IC50 value of 145 μM. (FIG. 1)

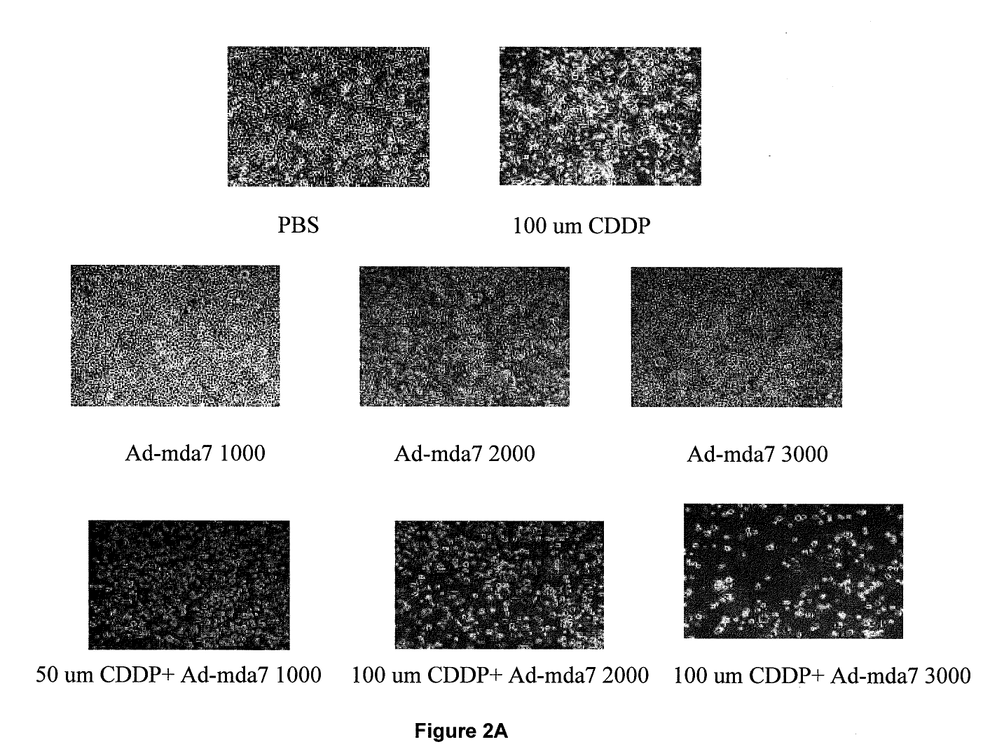

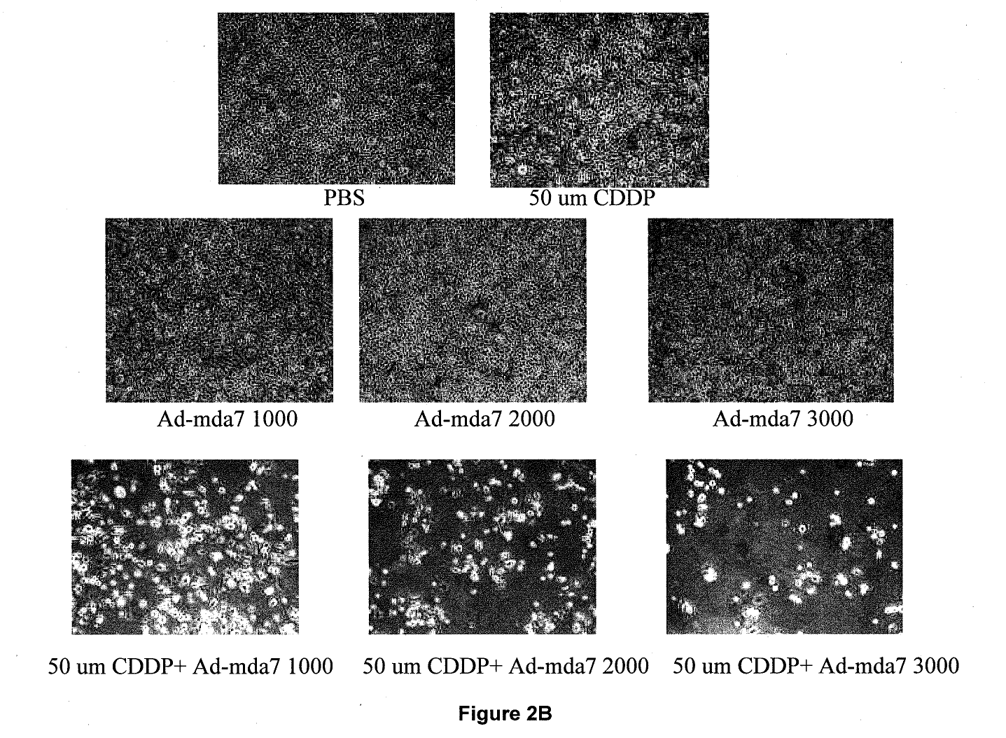

Ad-mda7 Plus CDDP Treatment Sensitizes CDDP-Resistant Tumor Cells. CDDP-resistant ovarian tumor (2008/C*13 R) cells were plated in six-well plates and treated with PBS, CDDP (50-100 μM) Ad-mda7 (1000, 2000, 3000 vp/cell), or with varying doses of Ad-mda7 plus a fixed dose of CDDP. Treatment schedule followed as follows: pretreatment with CDDP for 1 h, washed with PBS to remove any residual CDDP and then infect with Ad-mda7 for 3 h in serum free medium followed by replenishment of complete medium. Cells were observed at 72 h after treatment under bright-field microscopy. (FIG. 2A), CDDP used was 100 μM; (FIG. 2B), CDDP used was 50 μM. All other experimental conditions were the same in both experiments.

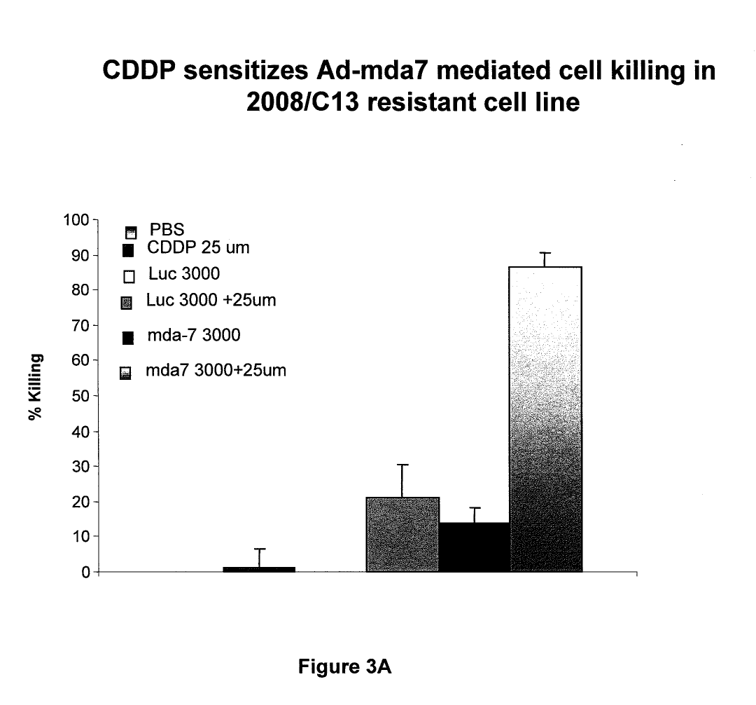

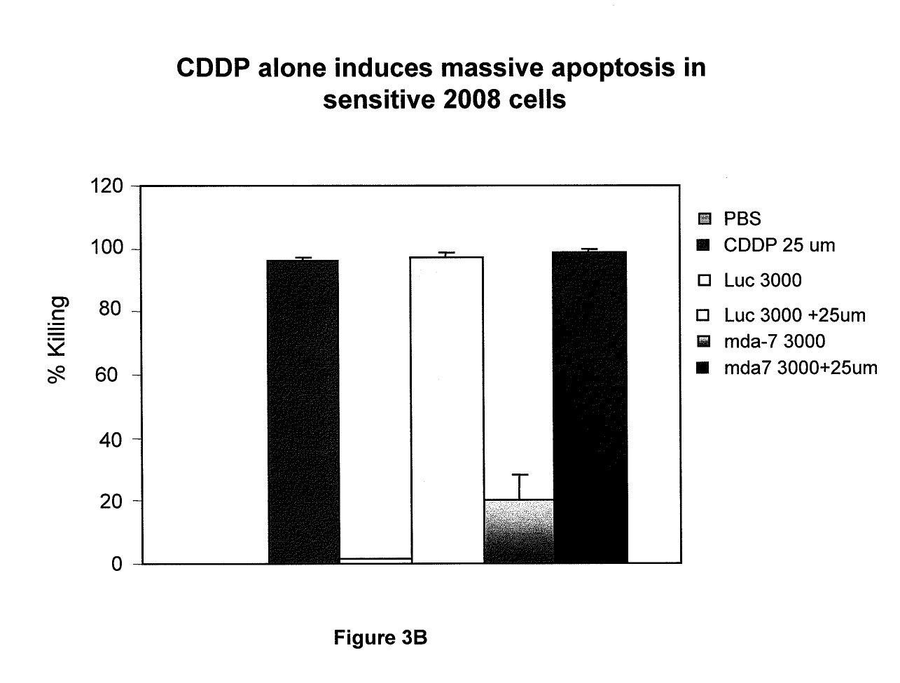

Next, CDDP-resistant (2008/C*13 R) and sensitive (2008) ovarian tumor cells were plated in six-well plates and treated with PBS, CDDP (25 μM) Ad-luc, Ad-mda7 (3000 vp/cell), Ad-mda7 plus CDDP or Ad-luc plus CDDP. Treatment schedule followed as follows: pretreatment with CDDP for 1 h, washed with PBS to remove any residual CDDP and then infect with Ad-mda7 for 3 h in serum free medium followed by replenishment of complete medium. Cells were harvested at 72 h after treatment and number of dead cells counted by the trypan blue assay. (FIG. 3A), CDDP-resistant 2008/C*13 R cells were significantly sensitized to the combined therapy of Ad-mda7 and CDDP compared to other treatment groups. (FIG. 3B) percent killing in 2008 cells was similar in CDDP-, Ad-luc plus CDDP and Ad-mda7 plus CDDP-treated cells suggesting the cells were highly sensitive to CDDP.

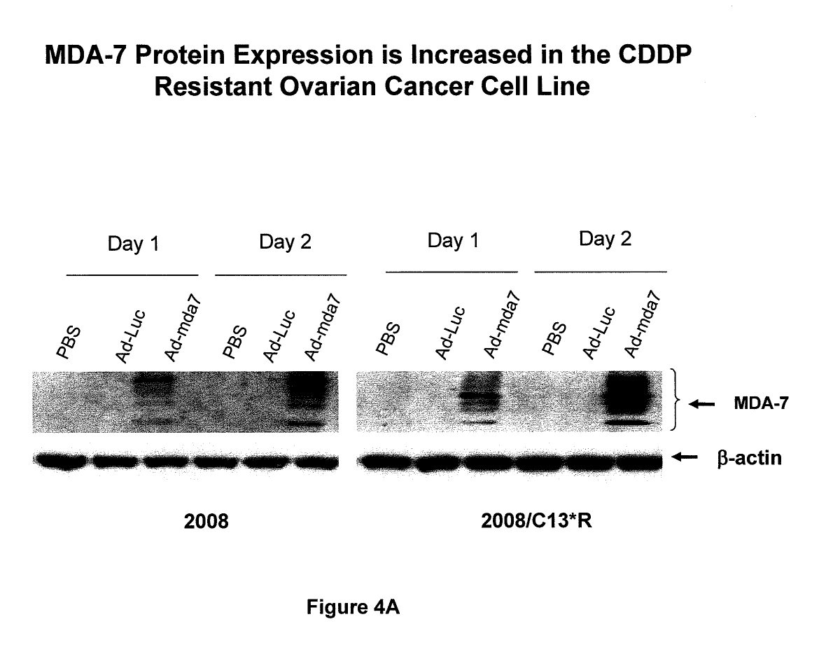

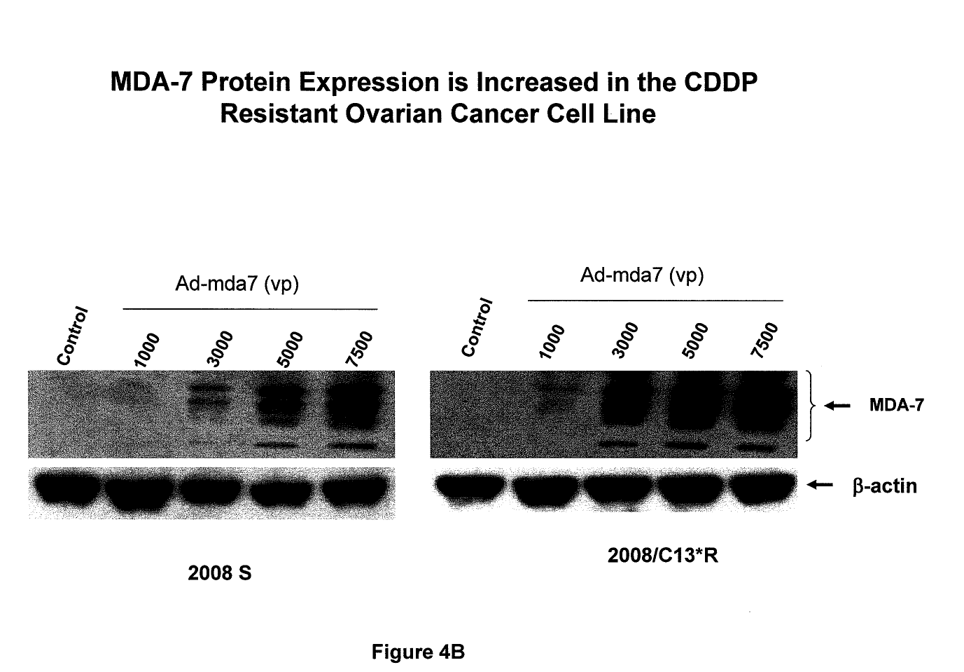

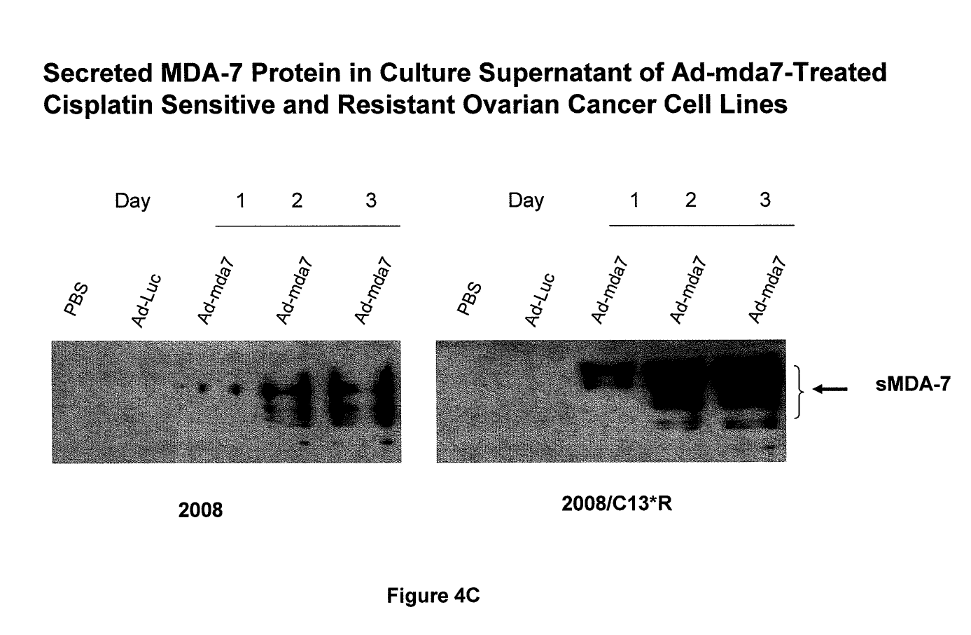

MDA-7 Protein Expression is Increased in CDDP-Resistant Ovarian Tumor Cells in a Time-dependent and Dose-Dependent Manner. Ovarian tumor cells (2008 and 2008/C*13 R) seeded in six-well plates were treated with PBS, Ad-luc or Ad-mda7 (3000 vp/cell) (FIG. 4A). Cells were harvested on day 1 and day 2 after treatment and analyzed for MDA-7 protein expression by western blotting. MDA-7 protein expression was observed in Ad-mda7-treated cells on both day 1 and day 2. However, MDA-7 protein expression levels was greatly increased in Ad-mda7-treated 2008/C*13 R cells compared to Ad-mda7-treated 2008 cells and in a time-dependent manner. Beta-actin was used as an internal loading control. Ovarian tumor cells (2008 and 2008/C*13 R) seeded in six-well plates were treated with different doses of Ad-mda7 (1000, 3000, 5000 or 7500 vp/cell) (FIG. 4B) Untreated cells served as control. Cells were harvested on day 2 after treatment and analyzed for MDA-7 protein expression by western blotting. MDA-7 protein expression was observed in Ad-mda7-treated cells in both 2008 and 2008/C*13 R cells. Expression was observed to increase in a dose-dependent manner. However, MDA-7 protein expression levels was greatly increased in Ad-mda7-treated 2008/C*13 R cells compared to Ad-mda7-treated 2008 cells at all doses. Beta-actin was used as an internal loading control. Analyses of tissue culture supernatant from Ad-mda7-treated 2008 and 2008/C*13 R cells showed MDA-7 protein that was increased over time (FIG. 4C). However, MDA-7 protein was higher in the supernatant of Ad-mda7-treated 2008/C*13 R cells compared to Ad-mda7-treated 2008 cells.

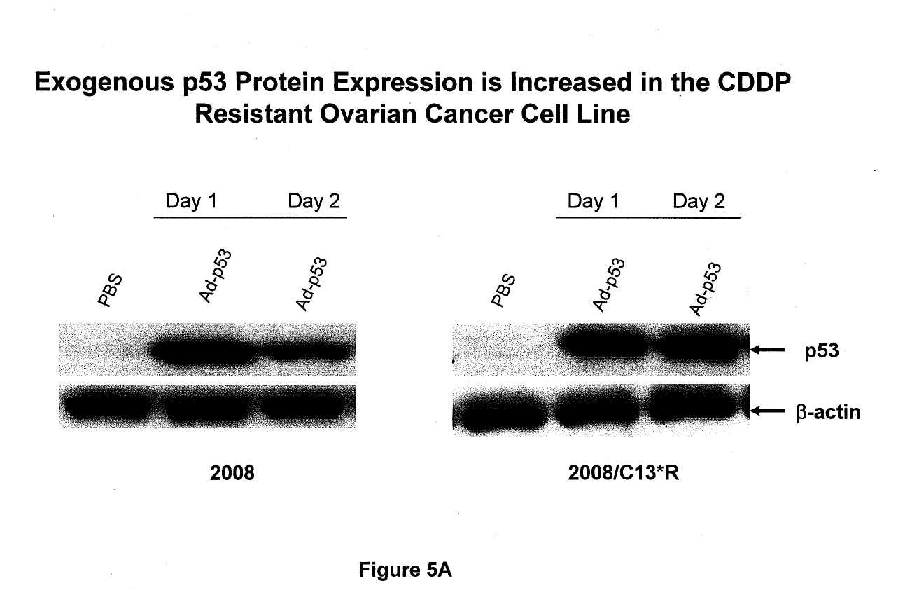

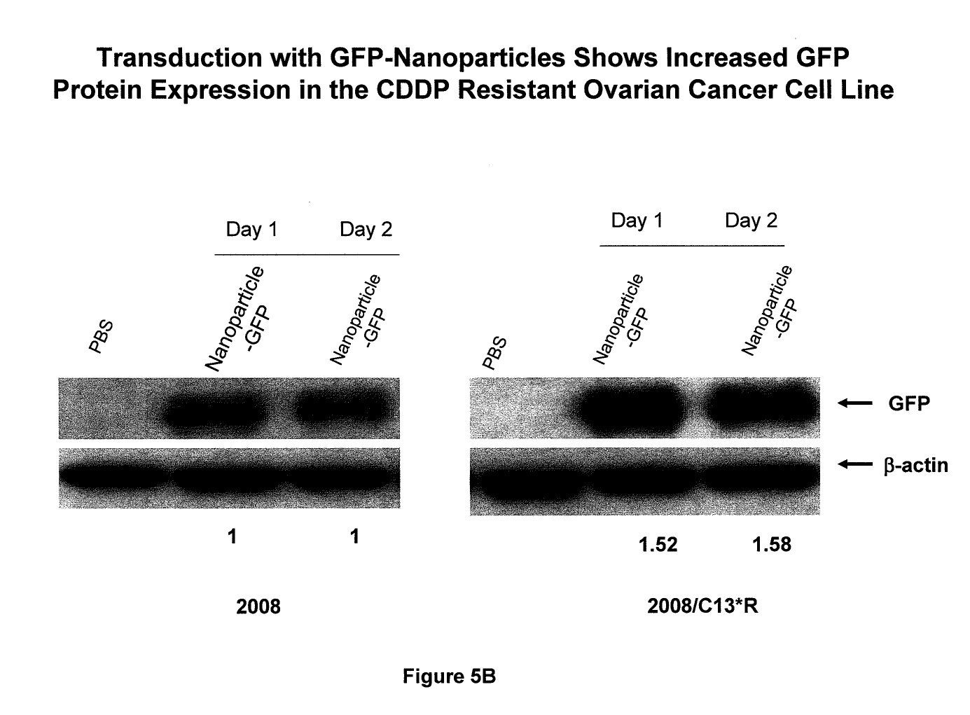

Exogenous p53 and GFP Protein Expression is Increased in CDDP-Resistant Ovarian Tumor Cells. Ovarian tumor cells (2008 and 2008/C*13 R) seeded in six-well plates were treated with PBS, Ad-p53 (3000 vp/cell), or Nanoparticle GFP (2.5 μg DNA). Cells were harvested on day 1 and day 2 after treatment and analyzed for transgene expression by western blotting. p53 protein expression was observed in Ad-p53-treated cells on both day 1 and day 2 (FIG. 5A). However, p53 protein expression levels was greatly increased/stabilized in Ad-p53-treated 2008/C*13 R cells compared to Ad-p53-treated 2008 cells. Beta-actin was used as an internal loading control. GFP protein expression was observed in Nanoparticle-GFP-treated cells on both day 1 and day 2 (FIG. 5B). However, GFP protein expression levels was greatly increased in Nanoparticle-GFP-treated 2008/C*13 R cells compared to Nanoparticle-GFP-treated 2008 cells.

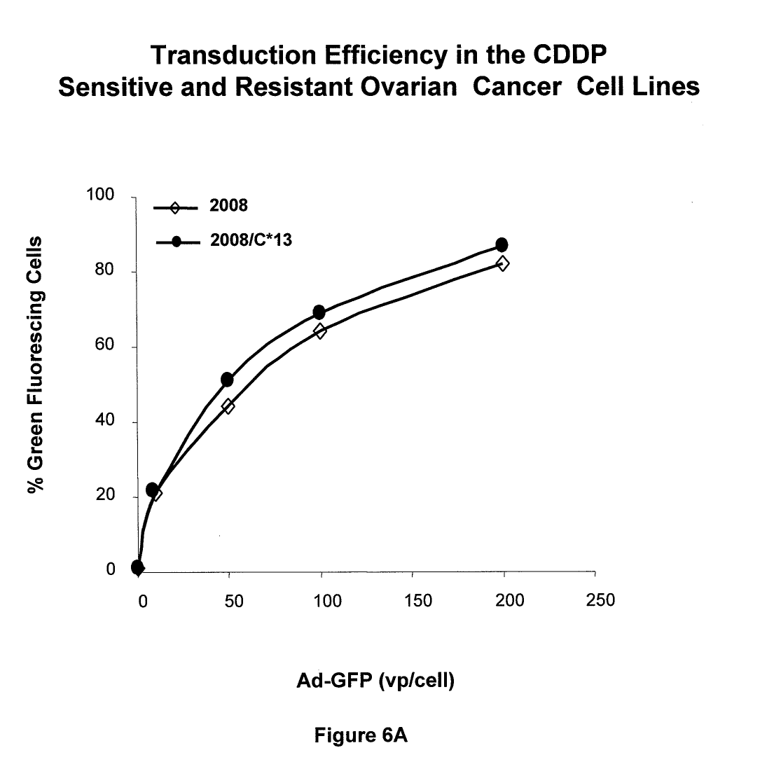

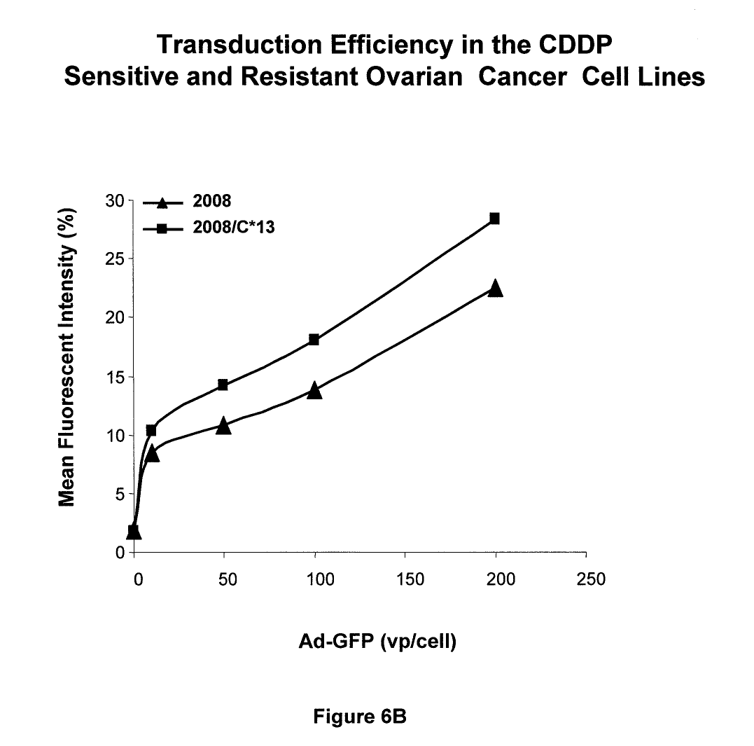

Transduction Efficiency in CDDP-Resistant Cells. Tumor cells (2008 and 2008/C*13 R) were treated with Ad-GFP at 10, 50, 100 and 200 vp/cell. Cells receiving no treatment served as control. At 24 h after treatment cells were harvested, washed with PBS three times, resuspended in 500 μl PBS and subjected to FACS analysis. A slight increase in number of GFP positive cells was observed in 2008/C*13 R cells compared to 2008 cells (FIG. 6A). A slight increase in GFP positive cells sorted by mean fluorescent intensity was also observed in 2008/C*13 R cells compared to 2008 cells (FIG. 6B).

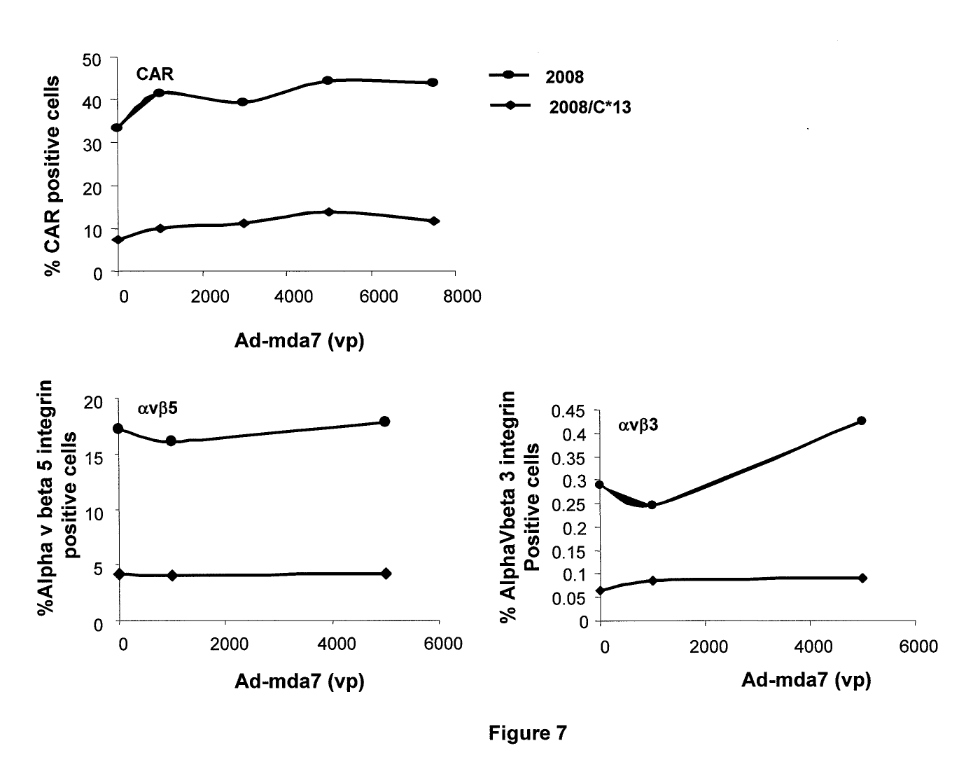

MDA-7 Protein Expression is not Increased in CDDP-Resistant Cells Due to Increase Adenovirus Receptor Expression. Tumor cells (2008 and 2008/C*13 R) were treated with Ad-mda-7 at 1000, 3000, 5000 and 7500 vp/cell. Cells receiving no treatment served as control. At 24 h after treatment cells were harvested, washed with PBS three times, resuspended in 500 μl PBS and incubated with FITC-conjugated-anti-CAR antibody, anti-alphaVbeta5 or anti-alphaVbeta3 antibody for 1 h. Cells were subsequently washed, resuspended and subjected to FACS analysis. The expression levels of CAR, alphaVbeta5 and alphaVbeta3 receptors was higher in 2008 cells compared to 2008/C*13 R cells. (FIG. 7).

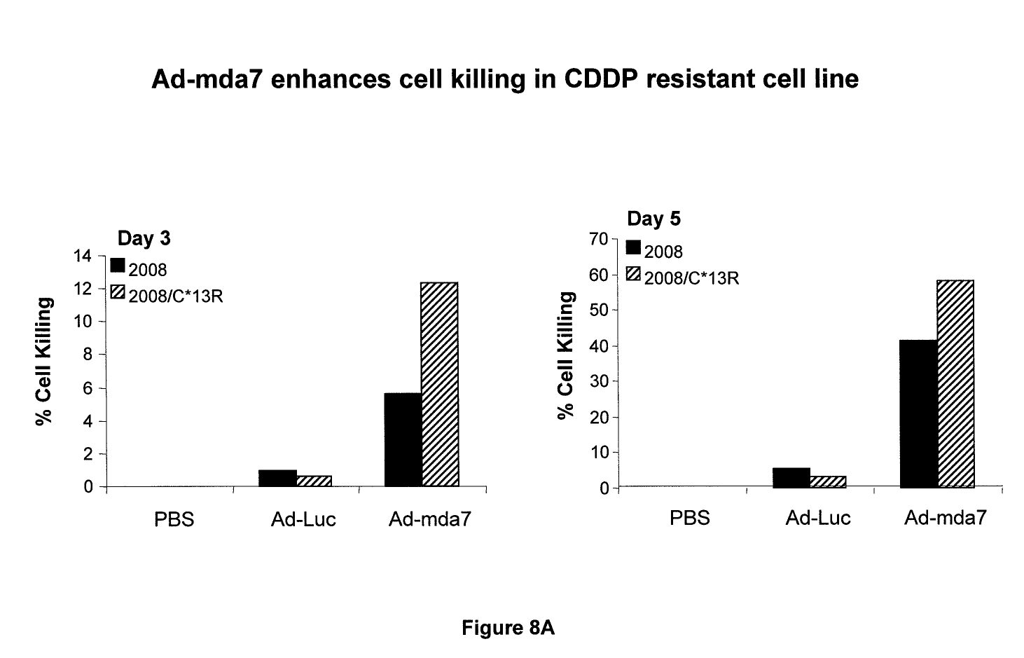

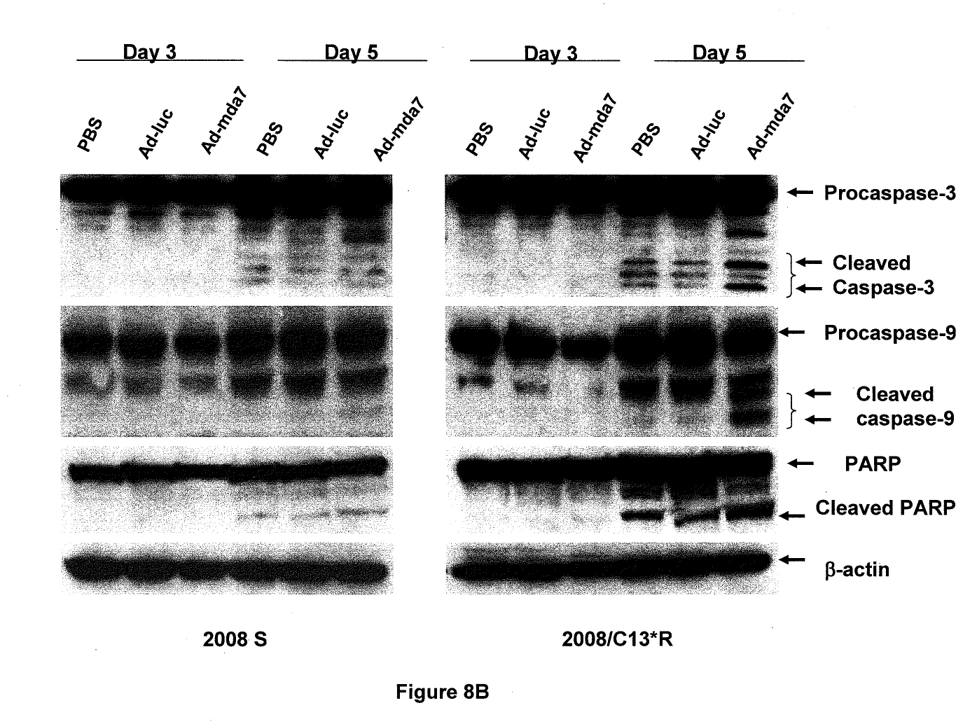

CDDP-Resistant Tumor Cells are More Sensitive to Ad-mda7. CDDP-resistant (2008/C*13 R) and sensitive (2008) ovarian tumor cells were plated in six-well plates and treated with PBS, Ad-luc or Ad-mda7 (3000 vp/cell). Treatment schedule followed was: treatment with Ad-mda7 for 3 h in serum free medium followed by replenishment of complete medium. Cells were harvested on day 3 and day 5 after treatment and number of dead cells counted by the trypan blue assay and analyzed for apoptotic markers by western blotting. CDDP-resistant 2008/C*13 R cells were significantly sensitized to Ad-mda7 as observed by the increase in the percent of killing compared to percent killing of 2008 cells (FIG. 8A). Increased Ad-mda7-mediated killing of 2008/C*13 R was observed at both time points. Western blotting analyses showed cleavage of caspase-3, caspase-9, and PARP on day 5 in both 2008/C*13 R and 2008 cells (FIG. 8B). However, the expression levels of these proteins were higher in 2008/C*13 R compared to 2008 cells.

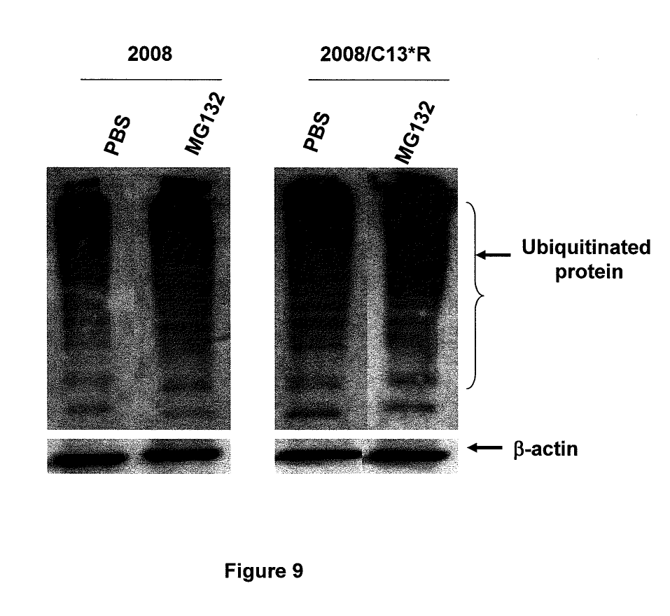

CDDP-Resistant Tumor Cells Have More Endogenous Ubiquitinated Proteins. CDDP-resistant (2008/C*13 R) and sensitive (2008) ovarian tumor cells were plated in six-well plates and were untreated or treated with MG132. Cells were harvested 24 h after treatment, total cell lysate prepared and subjected to western blotting. Membrane was probed with human anti-ubiquitinated antibody. Total ubiquitinated proteins was observed to be increased in CDDP-resistant 2008/C*13 R cells compared to 2008 cells. Treatment with MG132 however greatly increased the total ubiquitinated protein levels in 2008 but not in 2008/C*13 R cells. (FIG. 9)

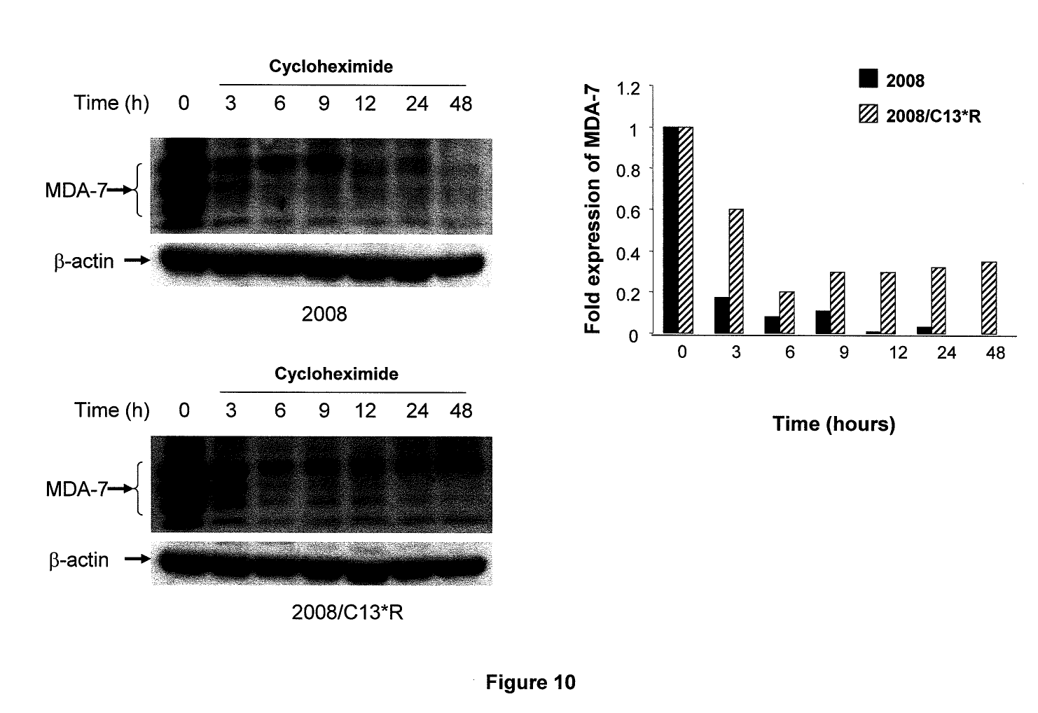

MDA-7 Protein Degradation is Delayed in CDDP-Resistant Tumor Cells. CDDP-resistant (2008/C*13 R) and sensitive (2008) ovarian tumor cells were plated in six-well plates and were treated with Ad-mda7. At 24 h after treatment cells were treated with Cyclohexamide. At 3, 6, 9, 12, 24 and 48 h after cyclohexamide treatment cells were harvested, lysates prepared and subjected to MDA-7 protein detection by western blotting. MDA-7 protein expression was detectable in both 2008 and 2008/C*13 R cells. However, the half-life of MDA-7 protein was observed to be increased and more than 48 h in CDDP-resistant 2008/C*13 R cells while 10-11 h in 2008 cells indicating delayed MDA-7 protein degradation in CDDP-resistant tumor cells. (FIG. 10)

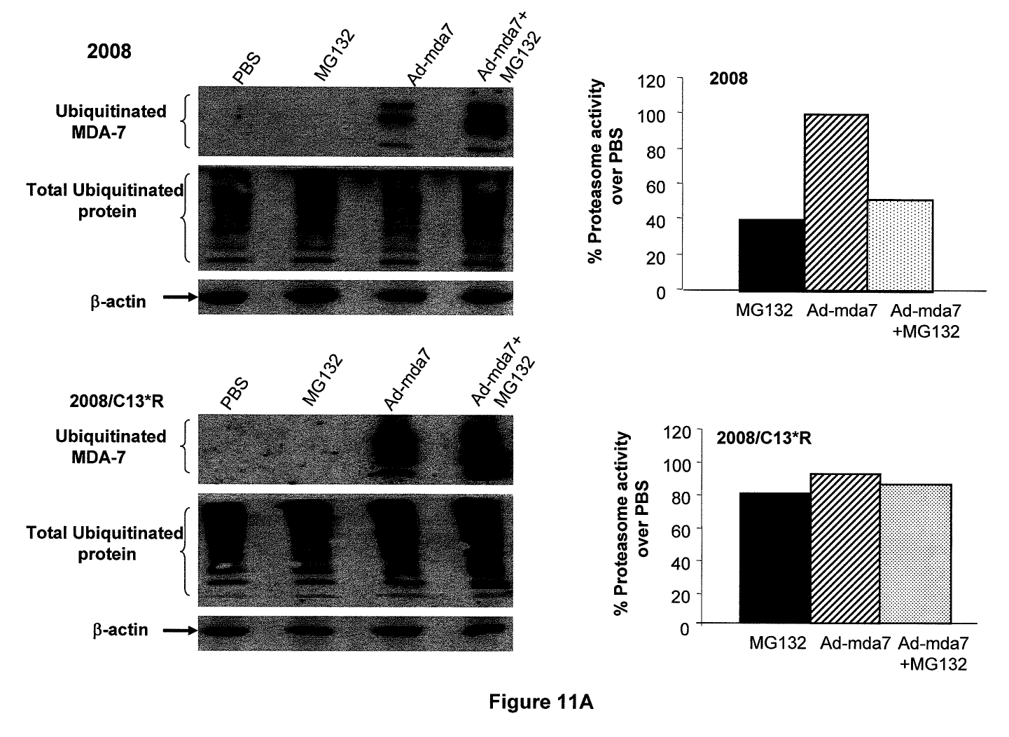

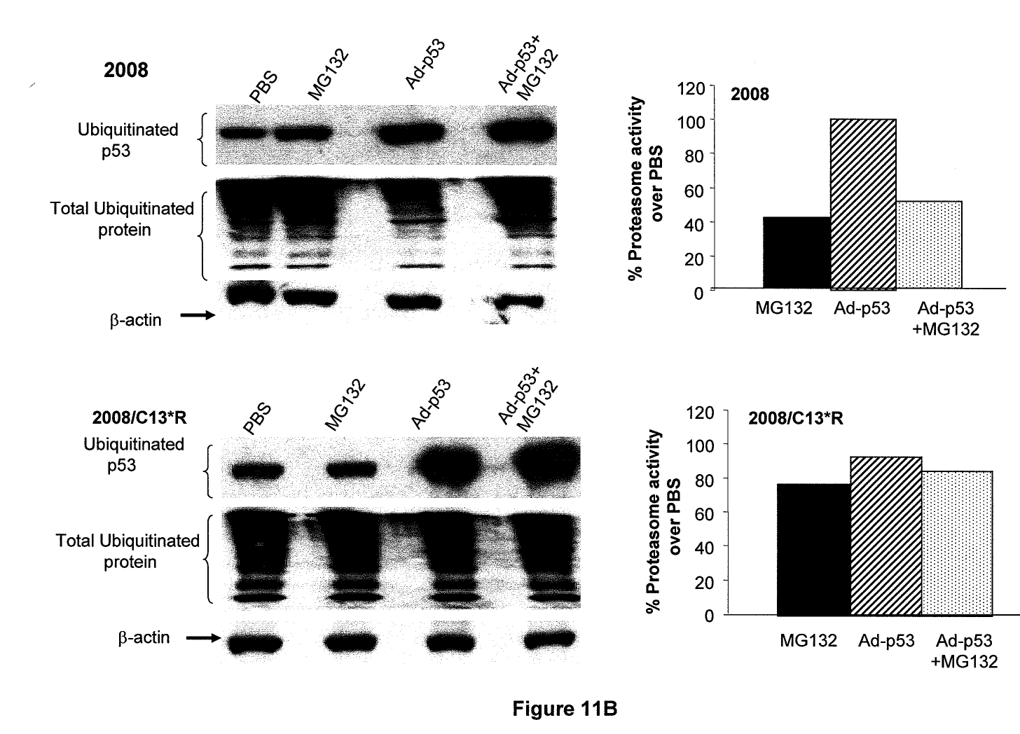

Inhibition of the Proteasomal Activity by MG132 Increases Ubiquitinated MDA-7 Protein Expression in CDDP-Sensitive Tumor Cells. CDDP-resistant (2008/C*13 R) and sensitive (2008) ovarian tumor cells were plated in six-well plates and were treated with PBS, MG132, Ad-mda7, or Ad-mda7 plus MG132 (FIG. 11A). At 48 h after treatment cells were harvested, lysates and supernatants prepared and subjected to MDA-7 and total ubiquitinated protein detection by western blotting and proteasome activity assay using commercially available kit. MDA-7 protein expression was detectable in Ad-mda7 treated 2008 and 2008/C*13 R cells with increased expression observed in 2008/C*13 R compared to 2008 cells. However, the MDA-7 protein was increased in 2008 cells when treated with MG132 plus Ad-mda7. In 2008/C*13 R cells, treatment with MG132 plus Ad-mda7 did not increase MDA-7 protein expression levels. Additionally, proteasome assay revealed MG132 effectively inhibited the proteasome activity in 2008 cells (60-65% inhibition) compared to that in 2008/C*13 R cells (20% inhibition) indicating CDDP-resistant cells have reduced proteasome activity. CDDP-resistant (2008/C*13 R) and sensitive (2008) ovarian tumor cells were plated in six-well plates and were treated with PBS, MG132, Ad-p53, or Ad-p53 plus MG132 (FIG. 11B). At 48 h after treatment cells were harvested, lysates and supernatants prepared and subjected to p53 protein detection by western blotting and proteasome activity using commercially available kit. P53 protein expression was detectable in Ad-p53-treated 2008 and 2008/C*13 R cells with increased expression observed in 2008/C*13 R compared to 2008 cells. However, the p53 protein was increased in 2008 cells when treated with MG132 plus Ad-p53. In 2008/C*13 R cells, treatment with MG132 plus Ad-p53 did not increase p53 protein expression levels. Additionally, proteasome assay revealed MG132 effectively inhibited the proteasome activity in 2008 cells (60-65% inhibition) compared to that in 2008/C*13 R cells (20% inhibition) indicating CDDP-resistant cells have reduced proteasome activity. Additionally, MDA-7 protein was demonstrated to be ubiquitinated.

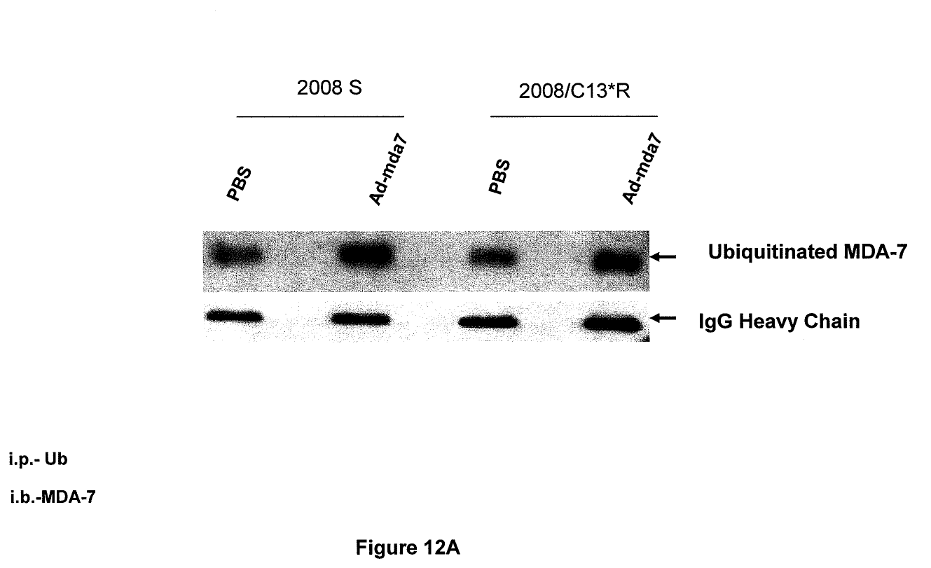

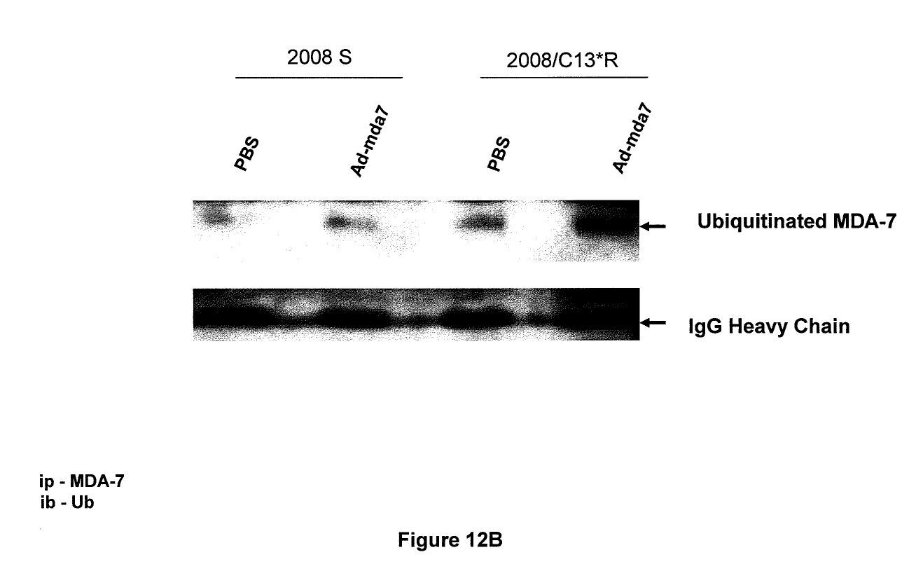

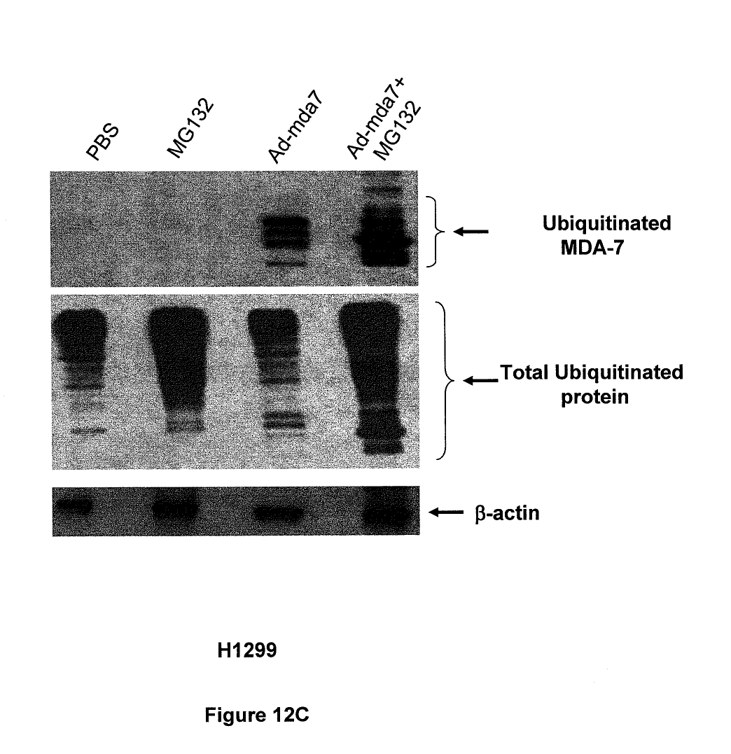

MDA-7 Protein is Ubiqutinated. CDDP-resistant (2008/C*13 R) and sensitive (2008) ovarian tumor cells were plated in six-well plates and were treated with PBS, or Ad-mda7. At 48 h after treatment cells were harvested, lysates prepared and subjected to immunoprecipitation using agarose-G-beads coated with anti-ubiquitin antibody (FIG. 12A), anti-MDA-7 antibody (FIG. 12B). Immunoprecipiates were run on a SDS-PAGE gel electrophoresis and probed with anti-MDA7 antibody (FIG. 12A) and anti-ubiquitin antibody (FIG. 12B). Human lung cancer cells (H1299) were treated with PBS, MG132, Ad-mda7 or Ad-mda7 plus MG132 (FIG. 12C). Cells were harvested at 24 h after treatment, lysates prepared and subjected to western blotting using human anti-ubiquitin antibody and anti-MDA-7 antibody. Detection of MDA-7 protein was observed in Ad-mda7 and Ad-mda7 plus MG132-treated cells. However, increased MDA-7 protein was detected in Ad-mda7 plus MG132-treated cells indicating MDA-7 protein is ubiquitinated.

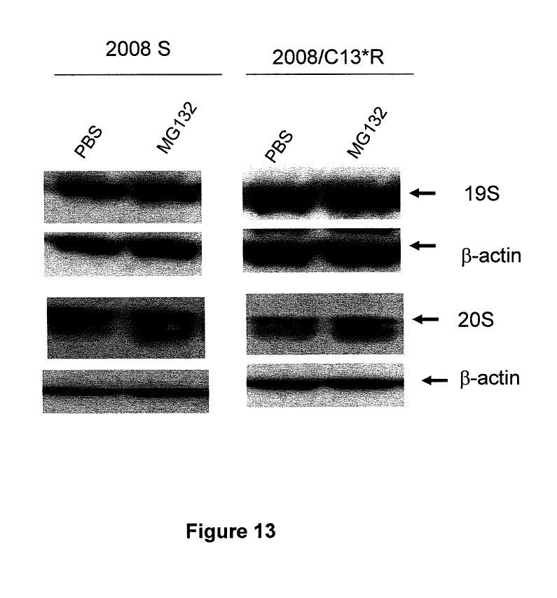

Expression of 20S Subunit but not 19S Subunit of the 26S Proteasome is Reduced in CDDP-Resistant Tumor Cells. CDDP-resistant (2008/C*13 R) and sensitive (2008) ovarian tumor cells were plated in six-well plates and were treated with PBS, or MG132. At 24 h after treatment cells were harvested, lysates prepared and subjected to western blotting. Primary antibodies used were anti-human 19S antibody and anti-human 20S antibody. No significant difference in the expression levels of 19S protein was observed in PBS or MG132-treated cells in both 2008 and 2008/C*13 R. However, expression levels of 20S protein was markedly reduced in 2008/C*13 R cells compared to 2008 cells both in PBS and MG132 treated cells indicating 20S expression is reduced in CDDP-resistant tumor cells. (FIG. 13).

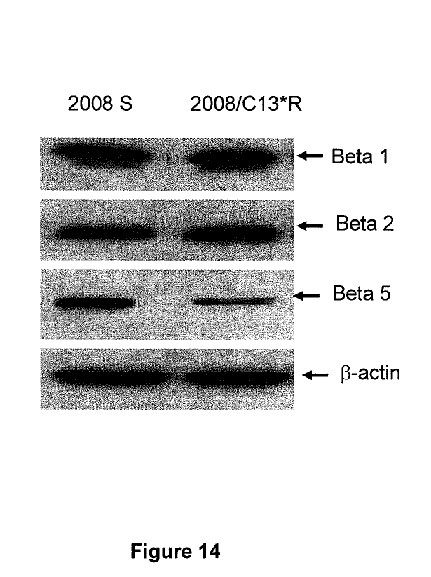

Expression of Beta-5 of the 20S Proteasome Subunit is Reduced in CDDP-Resistant Tumor Cells. CDDP-resistant (2008/C*13 R) and sensitive (2008) ovarian tumor cells were plated in six-well plates. At 24 h after plating cells were harvested, lysates prepared and subjected to western blotting. Primary antibodies used were anti-human Beta-1, Beta-2 and Beta-5 antibody. No significant difference in the expression levels of Beta-I and Beta-2 were observed between 2008 and 2008/C*13 R cells. However, expression levels of Beta-5 protein was markedly reduced in 2008/C*13 R cells compared to 2008 cells. Beta-actin was used as internal loading control. (FIG. 14).

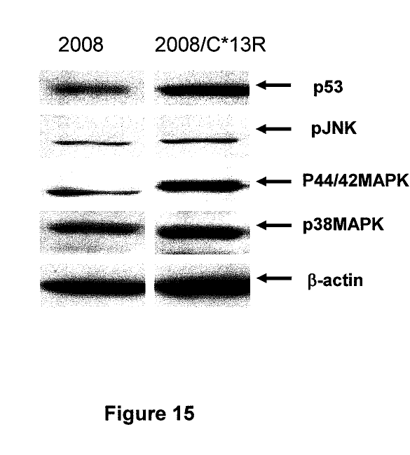

Expression of Endogenous Ubiquitinated Proteins is Increased in CDDP-Resistant Tumor Cells. CDDP-resistant (2008/C*13 R) and sensitive (2008) ovarian tumor cells were plated in six-well plates. At 24 h after plating cells were harvested, lysates prepared and subjected to western blotting. Primary antibodies used were anti-human p53, anti-human pJNK, antihuman-p38MAPK and anti-human p44/42MAPK antibody. Increased expression levels of p53, pJNK, p38MAPK, p44/42MAPK all of which undergo ubiquitination and proteasomal degradation were observed in 2008/C*13 R cells compared to the expression levels of these proteins in 2008 cells. Beta-actin was used as internal loading control. (FIG. 15)

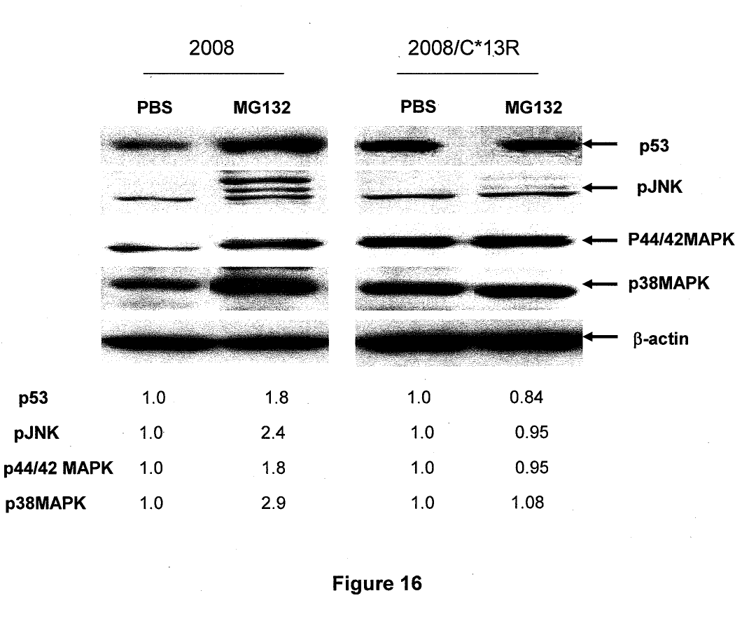

Expression of Endogenous Ubiquitinated Proteins is Increased in MG132-Treated CDDP-Sensitive Tumor Cells but not in CDDP-Resistant Tumor Cells. CDDP-resistant (2008/C*13 R) and sensitive (2008) ovarian tumor cells were plated in six-well plates. At 24 h after plating cells were treated with PBS or MG132. At 24 h after treatment cells were harvested, lysates prepared and subjected to western blotting. Primary antibodies used were anti-human p53, anti-human pJNK, antihuman-p38MAPK and anti-human p44/42MAPK antibody. Increased expression levels of p53, pJNK, p38MAPK, p44/42MAPK all of which undergo ubiquitination and proteasomal degradation were observed in 2008/C*13 R cells compared to the expression levels of these proteins in 2008 cells. However, treatment with MG132 resulted in increased expression of these proteins in 2008 cells but not in 2008/C*13 R cells indicating CDDP-resistant cells do not respond to proteasomal inhibitors. Beta-actin was used as internal loading control. (FIG. 16).

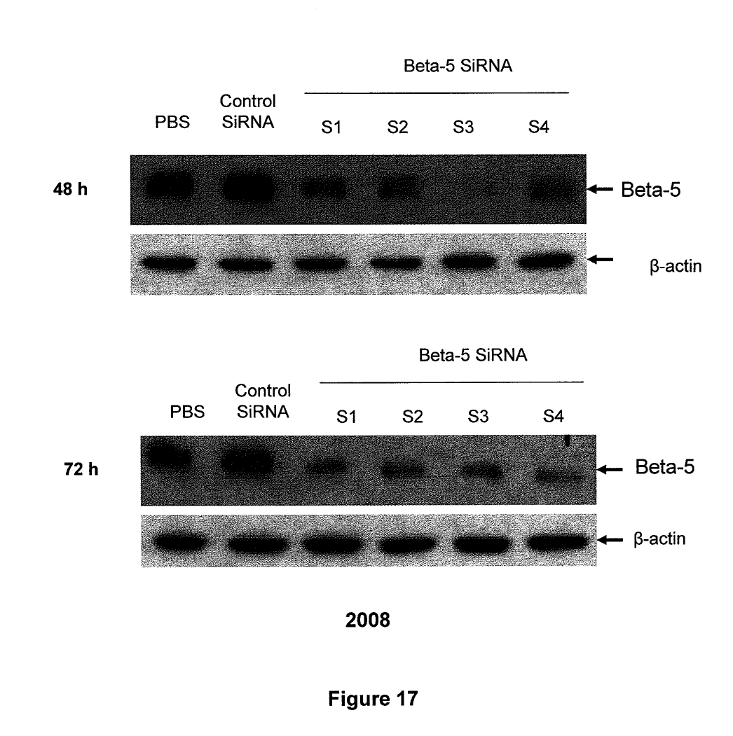

SiRNA-Mediated Inhibition of Beta-5 Subunit Expression of the 20S Proteasome in CDDP-Sensitive Tumor Cells. CDDP-sensitive (2008) ovarian tumor cells were plated in six-well plates. At 24 h after plating cells were treated with PBS, control SiRNA, or with four different beta-5 specific SiRNAs (100 nM). At 48 h and 72 h after treatment cells were harvested, lysates prepared and subjected to western blotting. Primary antibodies used were anti-human beta-5 antibody. A significant reduction in beta-5 protein expression was observed in cells treated with beta-5-specific SiRNA compared to PBS- and control SiRNA-treated cells. Beta-actin was used as internal loading control. (FIG. 17).

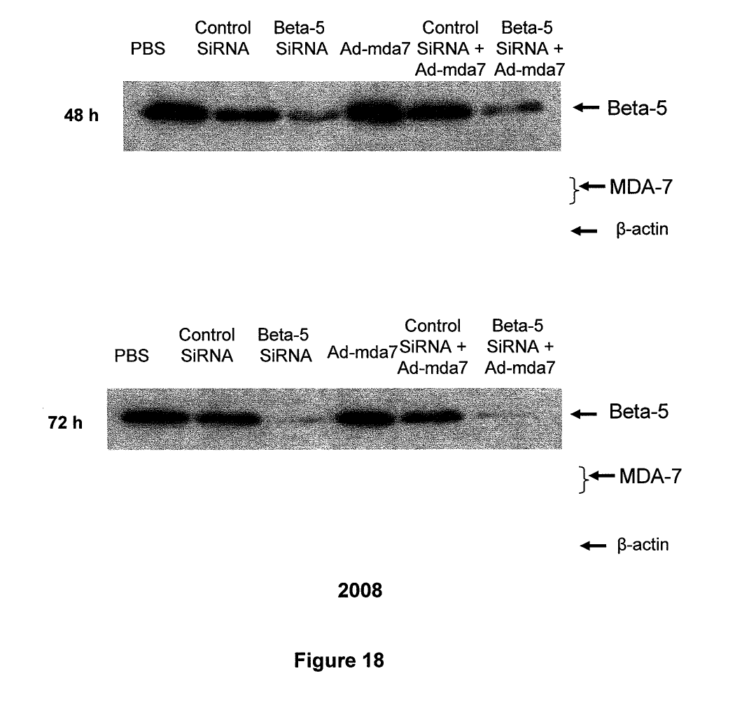

SiRNA-Mediated Inhibition of Beta-5 Subunit Expression of the 20S Proteasome in CDDP-Sensitive Tumor Cells Results in Increased MDA-7 Protein Expression. CDDP-sensitive (2008) ovarian tumor cells were plated in six-well plates. At 24 h after plating cells were treated with PBS, control siRNA, beta-5 specific siRNAs (100 nM), Ad-mda7, Ad-mda7 plus control siRNA or Ad-mda7 plus beta-5 specific siRNA. At 48 h and 72 h after treatment cells were harvested, lysates prepared and subjected to western blotting. Primary antibodies used were anti-human beta-5 antibody and anti-human MDA-7 antibody. A significant reduction in beta-5 protein expression was observed in cells treated with beta-5-specific siRNA compared to PBS- and control siRNA-treated cells. Correlating with inhibition of Beta-5 expression was the increase in MDA-7 protein expression. Beta-actin was used as internal loading control. (FIG. 18).

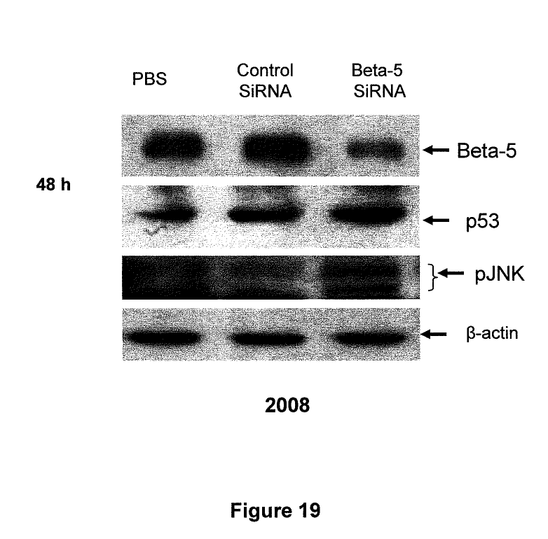

SiRNA-Mediated Inhibition of Beta-5 Subunit Expression of the 20S Proteasome in CDDP-Sensitive Tumor Cells Results in Increased Endogenous Protein Expression. CDDP-sensitive (2008) ovarian tumor cells were plated in six-well plates. At 24 h after plating cells were treated with PBS, control SiRNA, or beta-5 specific siRNAs (100 nM). At 48 h after treatment cells were harvested, lysates prepared and subjected to western blotting. Primary antibodies used were anti-human beta-5 antibody, anti-human p53 antibody and anti-human pJNK antibody. A significant reduction in beta-5 protein expression was observed in cells treated with beta-5-specific siRNA compared to PBS- and control siRNA-treated cells. Correlating with inhibition of Beta-5 expression was the increase in p53 and pJNK protein expression. Beta-actin was used as internal loading control. (FIG. 19).

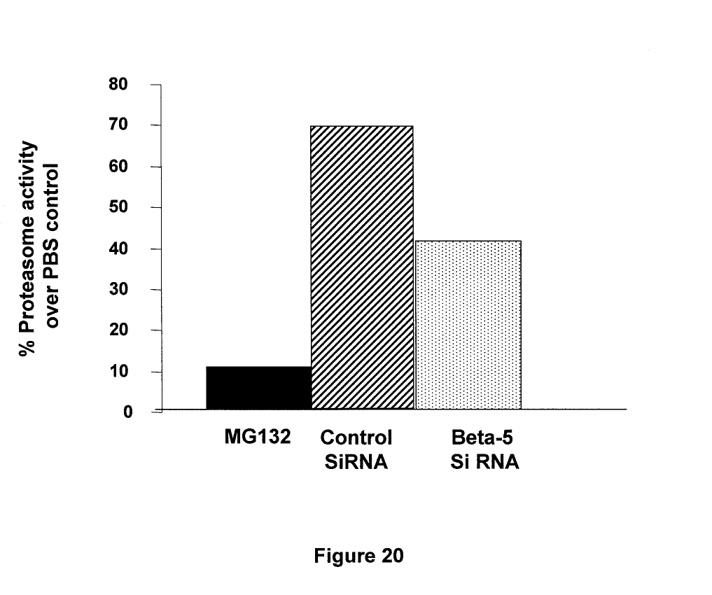

SiRNA-Mediated Inhibition of Beta-5 subunit expression of the 20S Proteasome in CDDP-Sensitive Tumor Cells Abrogates Proteasome Activity. CDDP-sensitive (2008) ovarian tumor cells were plated in six-well plates. At 24 h after plating cells were treated with PBS, MG132, control siRNA, or beta-5 specific siRNAs (100 nM). At 48 h after treatment cells were harvested, lysates prepared and subjected to proteasome activity assay using commercially available kit. A significant inhibition of proteasome activity was observed in Beta-5-siRNA-treated cells compared to control siRNA-treated cells. However, the inhibition of proteasome activity mediated by beta-5 siRNA was less than that observed in MG132-treated cells. (FIG. 20).

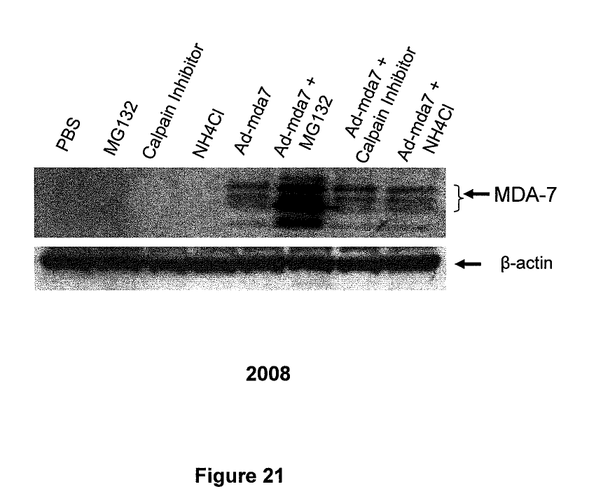

MDA-7 is Degraded by the 26S Proteasome and not by Other Proteases. CDDP-sensitive (2008) ovarian tumor cells were plated in six-well plates. At 24 h after plating cells were treated with PBS, MG132 (proteasome inhibitor), Calpain inhibitor (protease inhibitor), ammonium chloride (lysosome inhibitor), Ad-mda7, Ad-mda7 plus MG132, Ad-mda7 plus calpain inhibitor, Ad-mda7 plus ammonium chloride. At 24 h after treatment cells were harvested, lysates prepared and subjected to western blotting. Primary antibodies used were anti-human MDA-7 antibody. MDA-7 protein was detected in cells that were treated with Ad-mda7. However, a significant increase in MDA-7 protein expression was observed only in Ad-mda7 plus MG132-treated cells indicating MDa-7 undergoes proteasome-mediated degradation. Beta-actin was used as internal loading control. (FIG. 21).

Cisplatin-centered chemotherapy is a key treatment for ovarian cancer, but resistance to chemotherapeutic agents remains a major obstacle in treatment failure. Therefore, there is an obvious need for alternative approaches, such as gene therapy. The melanoma differentiation associated gene 7 (mda7) has the ability to work as a tumor suppressive gene in human cancer cells but not in normal cells. In this study, the growth inhibition induced by Ad-mda7 in 2008/C13*CDDP R (resistant cells) was significantly greater than that observed with 2008 CDDP S (sensitive cells) parental cell line. The expression of ectopic MDA-7 protein increased substantially in the CDDP resistant cell, whereas in the CDDP sensitive parental cell line MDA-7 protein expression decreased. Analysis of CAR receptor, αvβ3 and αvβ5 integrins in the CDDP resistant cells showed less endogenous levels and no increase in their levels when treated with Ad-mda7. On the other hand, a significant level of CAR receptor, αvβ3 and αvβ5 integrins in the CDDP sensitive cells were and no change in their levels treated with Ad-mda7 were found. The molecular mechanisms of increased adenoviral mediated MDA-7 expression in cisplatin-resistant human ovarian carcinoma cells 2008/C13*R as compared to parental cells 2008 S was investigated.

The ubiquitin-proteasome pathway plays a central role in the targeted destruction of cellular proteins, including cell cycle regulatory proteins and signal transduction proteins. Interestingly in the resistant cells, it was found that CDDP resistant cells has a defect in the ubiquitin/proteasome and markedly accelerated the endogenous expression of pp 38, pJNK, p44/42 MAPKs and p53 when compared to sensitive cells. Further, it was determined whether the defect in the ubiqutitin-proteasome pathway regulates the turnover of ectopic MDA-7 protein. Treatment of cells with the MG132, a proteasome inhibitor, showed that MDA-7 protein was ubiquitinated and its level significantly increased in CDDP sensitive and resistant cell lines. When the half-life of MDA-7 protein was examined, a remarkable stability was seen in CDDP resistant ovarian cancer cell lines 2008/C13*R, with a half life of 48 hr. This is sharply higher than the approximate 11 hr observed in CDDP sensitive cell lines 2008. This is associated with the activity of proteasome and levels of proteasome subunits 20S decreased in the CDDDP resistant cells when compared to CDDP sensitive cells. These observations provide a hint that the 20S proteasome subunit might be involved in controlling the accumulation of tumor suppressor MDA-7 protein that confers high sensitivity to cell killing. These results provide a novel mechanism for overcoming the apoptotic resistance of tumor cells by MDA-7 and accumulation of MDA-7 protein due to defect in the ubiquitin-proteasome pathway is a potential selective cancer therapeutic for life-threatening malignancies such as ovarian carcinoma.

Studies have shown that Ad-mda7 when combined with chemotherapy results in enhanced tumor killing. However whether Ad-mda7 can restore chemosensitivity to chemoresistant cells has not been previously studied. In the present study, we tested the ability of Ad-mda7 to restore chemosensitivity to CDDP resistant ovarian cancer cell line 2008/C13*R. Pretreatment of CDDP resistant tumor cells, first with CDDP followed by treatment with Ad-mda7, resulted in synergistic tumor killing, suggesting that Ad-mda7 restoration of sensitization to chemoresistance cells. It was found that CDDP can not only restore MDA-7 function in the resistant cells, but also significantly increase the protein level. Analysis of CAR receptor, αvβ3 and αvβ5 integrins in the CDDP resistant cells showed a less endogenous levels and no increase in their levels treated by Ad-mda7 in combination with cisplatin. On the other hand, it was found a significant level of CAR receptor, αvβ3 and αvβ5 integrins in the CDDP sensitive cells and no change in their levels treated by Ad-mda7 with cisplatin. Investigation into the underlying molecular mechanism showed a defect in the ubiquitin/proteasome pathway in the CDDP resistant cell line. These results show that a combination of Ad-mda7 and chemotherapeutic drugs an attractive modality for treating CDDP resistant ovarian cancer. These findings may have significant implications in human gene therapy using adenoviruses, especially in patients after unsuccessful cisplatin treatment.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 4,415,723
U.S. Pat. No. 4,458,066
U.S. Pat. No. 4,682,195
U.S. Pat. No. 4,683,202
U.S. Pat. No. 4,797,368
U.S. Pat. No. 4,870,287
U.S. Pat. No. 5,139,941
U.S. Pat. No. 5,187,260
U.S. Pat. No. 5,399,363
U.S. Pat. No. 5,466,468
U.S. Pat. No. 5,543,158
U.S. Pat. No. 5,641,515
U.S. Pat. No. 5,672,344
U.S. Pat. No. 5,693,617
U.S. Pat. No. 5,739,169
U.S. Pat. No. 5,760,395
U.S. Pat. No. 5,795,715
U.S. Pat. No. 5,801,005
U.S. Pat. No. 5,824,311
U.S. Pat. No. 5,830,880
U.S. Pat. No. 5,645,897
U.S. Pat. No. 5,846,225
U.S. Pat. No. 5,846,233
U.S. Pat. No. 5,846,945
U.S. Pat. No. 5,889,136
U.S. Pat. No. 5,925,565
U.S. Pat. No. 5,935,819
U.S. Pat. No. 6,410,010
U.S. application Ser. No. 09/351,778
U.S. application Ser. No. 09/575,473
U.S. application Ser. No. 09/615,154
U.S. application Ser. No. 10/017,472
U.S. application Ser. No. 10/378,590
U.S. application Ser. No. 10/391,068
U.S. application Ser. No. 10/791,692
U.S. Pub. No. 20020028785
U.S. Pub. No. 20040213764
U.S. Pub. No. 2004106539
U.S. Pub. No. 20050203047
U.S. Pub. No. 20050267037
EP 363,284
EP 363,284
EP 364,344
EP 393,457
PCT Appln. WO 00/05356
PCT Appln. WO 00/26368
PCT Appln. WO 00/44914
PCT Appln. WO 01/13904
PCT Appln. WO 01/36646
PCT Appln. WO 01/68836
PCT Appln. WO 02/08436
PCT Appln. WO 02/31168
PCT Appln. WO 02/85287
PCT Appln. WO 03/33029
PCT Appln. WO 88/10266
PCT Appln. WO 91/13904
PCT Appln. WO 92/12140
PCT Appln. WO 92/1850
PCT Appln. WO 98/07408
PCT Appln. WO 99/32619
Aksentijevich et al., Hum. Gene Ther., 7(9):1111-1122, 1996.
Angel et al., Cell, 49:729, 1987b.
Angel et al., Mol. Cell. Biol., 7:2256, 1987a.
Angelastro, et al., J. Med. Chem., 33(1):11-3, 1990.
Angliker et al., Biochem. J., 241(3):871-875, 1987.
Arap et al., Cancer Res., 55(6):1351-1354, 1995.
Atchison and Perry, Cell, 46:253, 1986.
Atchison and Perry, Cell, 48:121, 1987.
Austin-Ward and Villaseca, Revista Medica de Chile, 126(7):838-845, 1998.
Ausubel et al., In: Current Protocols in Molecular Biology, John, Wiley & Sons, Inc, New York, 1996.
Bajorin et al., J. Clin. Oncol., 6(5):786-792, 1988.
Bakhshi et al., Cell, 41(3):899-906, 1985.
Banerji et al., Cell, 27(2 Pt 1):299-308, 1981.
Banerji et al., Cell, 33(3):729-740, 1983.
Barber, Death Differ., 8:113-126, 2001.
Bates et al., Nature, 395:124-125, 1998.
Batterson and Roizman, J. Virol., 46(2):371-377, 1983.
Berkhout et al., Cell, 59:273-282, 1989.
Bernstein et al., Nature, 409:363-366, 2001.
Bianco, Expert Opin. Drug Deliv., 1(1):57-65, 2004.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Brennecke et al., Cell, 113:25-36, 2003.
Brignole et al., J. Nat'l. Cancer Inst., 98:1142-1157, 2006.
Buchhagen et al., Head and Neck, 18:529-537, 1996.
Bukowski et al., Clin. Cancer Res., 4(10):2337-2347, 1998.
Bulla and Siddiqui, J. Virol., 62:1437, 1986.
Caldas et al., Nat. Genet., 8(1):27-32, 1994.
Caley et al., J. Virology, 71(4):3031-3038, 1997.
Campbell and Villarreal, Mol. Cell. Biol., 8:1993, 1988.
Campere and Tilghman, Genes and Dev., 3:537, 1989.
Campo et al., Nature, 303:77, 1983.
Caplen et al., Gene, 252(1-2):95-105, 2000.
Casey et al., Oncogene, 6(10):1791-1797, 1991.
Celander and Haseltine, J. Virology, 61:269, 1987.
Celander et al., J. Virology, 62:1314, 1988.
Chada et al., Int. Immunopharmacol., 4:649-667, 2004.
Chada et al., Mol. Ther., 10:1085-1095, 2004.
Chang et al., Hepatology, 14:134A, 1991.
Chang et al., Mol. Cell. Biol., 9:2153, 1989.
Chatterjee et al., Proc. Natl. Acad. Sci. USA, 86:9114, 1989.
Cheng et al., Cancer Res., 54(21):5547-5551, 1994.
Choi et al., Cell, 53:519, 1988.
Christodoulides et al., Microbiology, 144(Pt 11):3027-3037, 1998.
Clark et al., Hum. Gene Ther., 6(10):1329-1341, 1995.
Clayman et al., Cancer Res., 55(14):1-6, 1995.
Clayman et al. J. Clin. Oncol., 16(6):2221-2232, 1998.
Cleary and Sklar, Proc. Natl. Acad. Sci. USA, (21):7439-7443, 1985.
Cleary et al., J. Exp. Med., 164(1):315-320, 1986.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394 403, 1963.
Cripe et al., EMBO J., 6:3745, 1987.
Culotta and Hamer, Mol. Cell. Biol., 9:1376, 1989.
Daly et al., Oncogene, 8:1721-1729, 1993.
Dandolo et al., J. Virology, 47:55-64, 1983.
Davidson et al., J. Immunother., 21(5):389-398, 1998.
Davis et al, Curr. Biol., 6:146-148, 1996.
DeLuca et al., J. Virol., 56(2):558-570, 1985.
Den Besten et al., Isr. Med. Assoc. J., 8:249-251, 2006.
Deschamps et al., Science, 230:1174-1177, 1985.
Dick et al., Biochem., 30(10):2725-2734, 1991.
Dillman, Cancer Biother. Radiopharm., 14(1):5-10, 1999.
Doench et al., Genes Dev., 17:438-42, 2003.
Doerr, Nat. Methods, 2(11):808, 2005.
Donze et al., EMBO J., 20:3771-3780, 2001.
Doyle, Semin. Oncol., 20(4):326-337, 1993.
Edbrooke et al., Mol. Cell. Biol., 9:1908, 1989.
Edlund et al., Science, 230:912-916, 1985.
Eicher et al., Clin. Cancer Res., 2(10): 1659-1664, 1996.
Elbashir et al., EMBO, 20(23):6877-6888, 2001.
Elbashir et al., Genes Dev., 5(2):188-200, 2001.
Elroy-Stein et al., Proc. Natl. Acad. Sci. USA, 86(16):6126-6130, 1989.
Ewoldt et al., Mol. Immunol., 29(6):713-721, 1992.
Felgner et al., Proc. Natl. Acad. Sci. USA, 84(21):7413-7417, 1987.
Feng and Holland, Nature, 334:6178, 1988.
Firak and Subramanian, Mol. Cell. Biol., 6:3667, 1986.
Fire et al., Nature, 391(6669):806-811, 1998.
Flotte and Carter, Gene Ther., 2(6):357-62, 1995.
Flotte et al., Proc. Natl. Acad. Sci. USA, 90(22): 10613-10617, 1993.
Flotte et al., Am. J. Respir. Cell Mol. Biol., 7(3):349-356, 1992.
Foecking and Hofstetter, Gene, 45(1):101-105, 1986.
Fraley et al., Proc. Natl. Acad. Sci. USA, 76:3348-3352, 1979.
Freifelder, In: Physical Biochemistry Applications to Biochemistry and Molecular Biology, 2nd Ed. Wm. Freeman and Co., NY, 1982.
Freshney, In: Animal Cell Culture, A Practical Approach, 2nd Ed., Oxford Press, UK, 1992.
Fujita et al., Cell, 49:357, 1987.
Gabizon et al., Cancer Res., 50(19):6371-6378, 1990.
Gazdar et al., In: Sym. Quant. Biol., Cold Spring Harbor, 59:565-572, 1994.
Gazdar et al., Intl. J. Cancer, 78:766-774, 1998.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh Choudhury et al., EMBO J., 6:1733 1739, 1987.
Gilles et al., Cell, 33:717, 1983.
Glorioso et al., Mol. Biotechnol., 4(1):87-99, 1995.
Gloss et al., EMBO J., 6:3735, 1987.
Godbout et al., Mol. Cell. Biol., 8:1169, 1988.
Goldberg et al., Nature, 357(6377):375-379, 1992.
Goldberg, Eur. J. Biochem., 203(1-2):9-23, 1992.
Gomez Foix et al., J. Biol. Chem., 267:25129 25134, 1992.
Goodbourn and Maniatis, Proc. Natl. Acad. Sci. USA, 85:1447, 1988.
Goodbourn et al., Cell, 45:601, 1986.
Gorunova et al., Genes Chrom. Cancer, 23:81-99, 1998.
Graham and Prevec, In: Methods in Molecular Biology: Gene Transfer and Expression Protocol, Murray (Ed.), Humana Press, NJ, 7:109 128, 1991.
Graham et al., J. Gen. Virl., 36(1):59-74, 1977.
Green et al., J. Biol. Chem., 256(4):1923-1928, 1981.
Greene et al., Immunology Today, 10:272, 1989
Grishok et al., Science, 287:2494-2497, 2000.
Grosschedl and Baltimore, Cell, 41:885, 1985.
Grunhaus and Horwitz, Seminar in Virology, 3:237-252, 1992.
Hanada et al., In: Proteinase Inhibitors: Medical and Biological Aspects, Katunuma et al. (Eds.), Springer-Verlag, 25-36, 1983.
Hanibuchi et al., Int. J. Cancer, 78(4):480-485, 1998.
Haslinger and Karin, Proc. Natl. Acad. Sci. USA, 82:8572, 1985.
Hauber and Cullen, J. Virology, 62:673, 1988.
Hellstrand et al., Acta Oncol., 37(4):347-353, 1998.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Hernandez et al., J. Med. Chem., 35(6):1121-1129, 1992.
Herr and Clarke, Cell, 45:461, 1986.
Hersdorffer et al., DNA Cell Biol., 9:713-723, 1990.
Herz and Gerard, Proc. Natl. Acad. Sci. USA, 90:2812 2816, 1993.
Hirano et al., Cell, 127:675-677, 2006.
Hirochika et al., J. Virol., 61:2599, 1987.
Hirsch et al., Mol. Cell. Biol., 10:1959, 1990.
Holbrook et al., Virology, 157:211, 1987.
Holland and Holland, J Biol Chem, 255(6):2596-605, 1980.
Hollstein et al., Science, 253:49-53, 1991.
Honess and Roizman, J. Virol., 14(1):8-19, 1974.
Honess and Roizman, J. Virol., 16(5):1308-130826. 1975.
Horlick and Benfield, Mol. Cell. Biol., 9:2396, 1989.
Horwich, et al., J. Virol., 64:642-650, 1990.
Huang et al., Cell, 27:245, 1981.
Hudig et al., J. Immunol., 147(4):1360-1368, 1991.
Hudig et al., Mol. Immunol., 26(8): 793-798, 1989.
Hug et al., Mol. Cell. Biol., 8:3065, 1988.
Hughson et al., Cancer Genet. Cytogenet., 106:93-104, 1998.
Hui and Hashimoto, Infect. Immun., 66(11):5329-5336, 1998.
Hung et al., JAMA, 273:1908, 1995.
Hussussian et al., Nat. Genet., 8(1):15-21, 1994.
Hutvagner and Zamore, Science, 297(5589):2056-2060, 2002.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Inouye and Inouye, Nucleic Acids Res., 13:3101-3109, 1985.
Irie and Morton, Proc. Natl. Acad. Sci. USA, 83(22):8694-8698, 1986.
Irie et al., Lancet., 1(8641):786-787, 1989.
Ito et al., Cancer Gene Therapy, 11:733-739, 2004.
Jagus et al., Int. J. Biochem. Cell. Biol., 31:123-138, 1999.
Jakobovits et al., Mol. Cell. Biol., 8:2555, 1988.
Jaynes et al., Mol. Cell. Biol., 8:62, 1988.
Jemal et al, Cancer J. Clin., 52:23-47, 2002.
Ji et al., Cancer Res., 62:2715-2720, 2000.
Jiang et al., Proc. Natl. Acad. Sci. USA, 93(17):9160-9165, 1996.
Johnson et al., Mol. Cell. Biol., 9:3393, 1989.
Jones and Shenk, Cell, 13:181 188, 1978.
Ju et al., Gene Ther., 7(19):1672-1679, 2000.
Kadesch and Berg, Mol. Cell. Biol., 6:2593, 1986.
Kajiwara et al., Biochem. Int., 15(5):935-944, 1987.
Karin et al., Biochem., 27(7):2547, 1988.
Karin et al., Thrombosis and Haemostasis, 64(1):133-137, 1990.
Karin et al., Nat. Genet., 8(1):23-26, 1994.
Kaneda et al., Science, 243:375-378, 1989.
Kaplitt et al., Nat. Genet., 8(2):148-154, 1994.
Karin et al., Mol. Cell. Biol., 7:606, 1987.
Karlsson et al., EMBO J., 5:2377 2385, 1986.
Katinka et al., Cell, 20:393, 1980.
Kato et al, J. Biol. Chem., 266:33613364, 1991.
Kawamoto et al., Mol. Cell. Biol., 8:267, 1988.
Kennedy and Schultz, Biochemistry, 18(2):349-356, 1979.
Kersemaekers et al., Intl. J. Cancer, 79:411-417, 1998. 1998;
Ketting et al., Cell, 99(2):133-141, 1999.
Ketting et al., Genes Dev., 15(20):2654-2659, 2001.
Kiledjian et al., Mol. Cell. Biol., 8:145, 1988.
Klamut et al., Mol. Cell. Biol., 10: 193, 1990.
Knight and Bass, Science, 2:2, 2001.
Koch et al., Mol. Cell. Biol., 9:303, 1989.
Kohno et al., Cancer, 85:341-347, 1999.
Kotin et al., Proc. Natl. Acad. Sci. USA, 87(6):2211-2215, 1990.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
LaFace et al., Virology, 162(2):483-486, 1988.
Larsen et al., Proc Natl. Acad. Sci. USA., 83:8283, 1986.
Laspia et al., Cell, 59:283, 1989.
Latimer et al., Mol. Cell. Biol., 10:760, 1990.
Laughlin et al., J. Virol., 60(2):515-524, 1986.
Lebkowski et al., Mol. Cell. Biol., 8(10):3988-3996, 1988.
Le Gal La Salle et al., Science, 259:988 990, 1993.
Lee et al., Nature, 294:228, 1981.
Lee et al., Nucleic Acids Res., 12:4191-206, 1984.
Levrero et al., Gene, 101:195 202, 1991.
Lin and Avery, Nature, 402:128-129, 1999.
Lin et al., Mol. Cell. Biol., 10:850, 1990.
Liu et al., Cancer Res., 55(14):3117-3122, 1995.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Mabry et al., In: Lung Cancer in the Genetic Basis of Human Cancer, Vogelstein and Kinzler (Eds.), McGraw Hill, 671-679, 1998.
Macejak and Sarnow, Nature, 353:90-94, 1991.
Majors and Varmus, Proc. Natl. Acad. Sci. USA, 80:5866, 1983.
Maloney and Workman, Expert Opin. Biol. Ther., 2:3-24, 2002.
Mann et al., Cell, 33:153-159, 1983.
Markowitz et al., J. Virol., 62:1120 1124, 1988.
McCarty et al., J. Virol., 65(6):2936-2945, 1991.
McKenzie et al., Surgery, 136:437-442, 2004.
McLaughlin et al., J. Virol., 62(6):1963-1973, 1988.
McNeall et al., Gene, 76:81, 1989.
Mhashilkar et al., Mol. Med., 7:271-282, 2001.
Mhashilkar et al., Mol. Ther., 8:207-219, 2003.
Miksicek et al., Cell, 46:203, 1986.
Minna et al., In: Cancer: Principles and Practice of Oncology, 5th Ed., Philadelphia: Lippincott, 849-857, 1997.
Mitchell et al., Ann. NY Acad. Sci., 690:153-166, 1993.
Mitchell et al., J. Clin. Oncol., 8(5):856-869, 1990.
Montgomery et al., Proc. Natl. Acad. Sci. USA, 95:15502-15507, 1998.
Mordacq and Linzer, Genes and Dev., 3:760, 1989.
Moreau et al., Nucl. Acids Res., 9:6047, 1981.
Mori et al., Cancer Res., 54(13):3396-3397, 1994.
Morton et al., Arch. Surg., 127:392 399, 1992.
Mourelatos et al., Genes Dev., 16(6):720-728, 2002.
Muesing et al., Cell, 48:691, 1987.
Murata, IUBMB Life, 58:344-348, 2006.
Muzyczka, Curr. Topics Microbiol. Immunol., 158:97-129, 1992.
Myung et al., Medicinal Research Reviews, 21:245-273, 2001
Neuberger et al., Nucleic Acids Res., 16(14B):6713-6724, 1988.
Ng et al., Nuc. Acids Res., 17:601, 1989.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nobri et al., Nature (London), 368:753-756, 1995.
Odaka et al., Biochem., 30(8):2217-2227, 1991.
Ohi et al., Gene, 89(2):279-282, 1990.
Okamoto et al., Proc. Natl. Acad. Sci. USA, 91(23):11045-11049, 1994.
Ondek et al., EMBO J., 6:1017, 1987.
Orlow et al., Cancer Res, 54(11):2848-2851, 1994.
Orlowski et al., Arch. Biochem. and Biophys., 269(1):125, 1989.
Orlowski, Biochemistry, 29(45):10289-10297, 1990.
Ornitz et al., Mol. Cell. Biol., 7:3466, 1987.
Oweida et al., Thrombosis Res., 58(2):391, 1990.
Palmiter et al., Nature, 300:611, 1982.
Parkes et al., Biochem. J., 230(2):509-516, 1985.
Pataer et al., Cancer Res., 62:2239-2243, 2002.
Paskind et al., Virology, 67:242-248, 1975.
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Peto et al., Lancet, 355:1822, 2000.
Picard and Schaffner, Nature, 307:83, 1984.
Pietras et al., Oncogene, 17(17):2235-2249, 1998.
Pinkert et al., Genes and Dev., 1:268, 1987.
Pisani et al., Int. J. Cancer, 83:18-29; 870-873, 1999.
Ponta et al., Proc. Natl. Acad. Sci. USA, 82:1020, 1985.
Post et al., Cell, 24(2):555-65, 1981.
Powers et al., Biochem., 29(12):3108, 1990.
Powers et al., In: Proteinase Inhibitors, Barrett et al. (Eds.), Elsevier, 55-152, 1986.
Powers et al., J. Cell. Biochem., 39(1):33-46, 1989.
Puri et al., Arch. Biochem. Biophys., 271(2):346-358, 1989.
Qin et al., Proc. Natl. Acad. Sci. USA, 95(24):14411-14416, 1998.
Queen and Baltimore, Cell, 35:741, 1983.
Quinn et al., Mol. Cell. Biol., 9:4713, 1989.
Racher et al., Biotechnology Techniques, 9:169 174, 1995.
Ragot et al., Nature, 361:647 650, 1993.
Rao et al., Thromb. Res., 47:635, 1987.
Ravindranath and Morton, Intern. Rev. Immunol., 7: 303 329, 1991.
Redondo et al., Science, 247:1225, 1990.
Reinhart et al., Nature, 403:901-906, 2000.
Reisman and Rotter, Mol. Cell. Biol., 9:3571, 1989.
Remington's Pharmaceutical Sciences, 15th ed., pages 1035-1038 and 1570-1580, Mack Publishing Company, Easton, Pa., 1980.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Rippe et al., Mol. Cell. Biol., 9(5):2224-22277, 1989.
Rittling et al., Nuc. Acids Res., 17:1619, 1989.
Rivett et al., Archs. Biochem. Biophys., 268(1):1-8, 1989.
Rivett et al., J. Biol. Chem., 264(21):12215-12219, 1989.
Rock and Goldberg, Annu. Rev. Immunol., 17:739-737, 1999.
Rosenberg et al., Ann. Surg. 210(4):474-548, 1989.
Rosenberg et al., Nature Med., 4:321-327, 1998.
Rosenfeld et al., Science, 252:431-434, 1991.
Roth et al., Nat. Med., 2:985-991, 1996.
Roth, Forum, 8:368-376, 1998.
Sambrook et al., In: Molecular cloning, Cold Spring Harbor Laboratory Press, Cold Spring Harbor, N.Y., 2001.
Samulski et al., J. Virol., 63:3822-3828, 1989.
Samulski et al., EMBO J., 3941-3950, 1991.
Sarkar et al., Biotechniques Suppl., 30-39, 2002.
Saxena et al, J. Biol. Chem., 278(45):44312-44319, 2003.
Schaffner et al., J. Mol. Biol., 201:81, 1988.
Scheit, In: Synthesis and Biological Function, Wiley-Interscience, NY, 171-172, 1980.
Searle et al., Mol. Cell. Biol., 5:1480, 1985.
Sekido et al., Proc. Natl. Acad. Sci. USA, 93:4120-4125, 1996.
Sekido et al., Oncogene, 16:3151-3157, 1998.
Serrano et al., Nature, 366:704-707, 1993.
Serrano et al., Science, 267(5195):249-252, 1995.
Sharp and Marciniak, Cell, 59:229, 1989.
Sharp and Zamore, Science, 287:2431-2433, 2000.
Sharp, Genes Dev., 13:139-141, 1999.
Shaul and Ben-Levy, EMBO J., 6:1913, 1987.
Shelling and Smith, Gene Therapy, 1:165-169, 1994.
Sherman et al., Mol. Cell. Biol., 9:50, 1989.
Sleigh and Lockett, J. EMBO, 4:3831, 1985.
Smith and Moss, Gene, 25(1):21-8, 1983.
Smyth-Templeton et al., DNA Cell Biol., 21(12):857-867, 1997.
Spalholz et al., Cell, 42:183, 1985.
Solodin et al., Biochemistry, 34(41):13537-13544, 1995.
Spandau and Lee, J. Virology, 62:427, 1988.
Spandidos and Wilkie, EMBO J., 2:1193, 1983.
Stephens and Hentschel, Biochem. J., 248:1, 1987.
Stratford Perricaudet and Perricaudet, In: Human Gene Transfer, Eds, Cohen Haguenauer and Boiron, John Libbey Eurotext, France, 5161, 1991.
Stratford-Perricaudet et al., Hum. Gene. Ther., 1:241-256, 1990.
Stuart et al., Nature, 317:828, 1985.
Su et al., Cancer Res., 58, 2339-2342, 1998.
Su et al., Proc. Natl. Acad. Sci. USA, 95:14400-14405, 1998.
Sullivan and Peterlin, Mol. Cell. Biol., 7:3315, 1987.
Swartzendruber and Lehman, J. Cell. Physiology, 85:179, 1975.
Tabara et al., Cell, 99(2): 123-132, 1999.
Takebe et al., Mol. Cell. Biol., 8:466, 1988.
Tanaka et al., New Biol., 4(3): 173-187, 1992.
Tang et al., Blood, 104(9):2704-2713, 2004.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Taylor et al., Science, 285:107-110, 1999.
Temin, In: Gene Transfer, Kucherlapati (Ed.), NY, Plenum Press, 149-188, 1986.
Thierry et al., Proc. Natl. Acad. Sci. USA, 92(21):9742-9746, 1995.
Thiesen et al., J. Virology, 62:614, 1988.
Top et al., J. Infect. Dis., 124:155-160, 1971.
Tratschin et al., Mol. Cell. Biol., 4:2072-2081, 1984.
Tratschin et al., Mol. Cell. Biol., 5:3258-3260, 1985.
Treisman, Cell, 42:889, 1985.
Tronche et al., Mol. Biol. Med., 7:173, 1990.
Tronche et al., Mol. Cell. Biol., 9(11):4759-4766, 1989.
Trotman et al., Cell, 128:141-156, 2007.
Trudel and Constantini, Genes and Dev., 6:954, 1987.
Tsujimoto and Croce, Proc. Natl. Acad. Sci. USA, 83(14):5214-5218, 1986.
Tsujimoto et al., Science, 228(4706):1440-1443, 1985.
Tsujinaka et al., Biochem. Biophys. Res. Commun., 153:1201, 1988.
Tsukamoto et al., Nat. Genet., 9(3):243-248, 1995.
Tyndell et al., Nuc. Acids. Res., 9:6231, 1981.
Uzawa et al., Cancer Genet. Cytogenet., 107:125-131, 1998.
Vasseur et al., Proc Natl. Acad. Sci. USA, 77:1068, 1980.
Vijayalakshmi et al., Biochem., 30(8):2175-2183, 1991.
Vinitsky et al., Biochemistry, 31(39):9421-9428, 1992.
Vlasak et al., J. Virol., 63(5):2056-2062, 1989.
Vogelstein et al., Nature, 408(6810):307-310, 2000.
Walsh et al., J. Clin. Invest, 94:1440-1448, 1994.
Wang and Calame, Cell, 47:241, 1986.
Wang et al., 2007;
Weber et al., Cell, 36:983, 1984.
Wei et al., Gene Therapy, 1:261-268, 1994.
Weinberg, Science, 254:1138-1146, 1991.
Williams, Sci STKE, 89:RE2, 2001.
Wincott et al., Nucleic Acids Res., 23(14):2677-2684, 1995.
Winoto and Baltimore, Cell, 59:649, 1989.
Wistuba et al., Cancer Res., 57:3154-3158, 1997.
Wistuba et al., Cancer Res., 59:1973-1979, 1999
Wistuba et al., Oncogene, 18:643-650, 1999.
Wong et al., Gene, 10:87 94, 1980.
Xu et al., Curr. Biol., 13:790-795, 2003.
Yamasaki et al., 2007
Yang and Huang, Gene Therapy, 4 (9):950-960, 1997.
Yang et al., J. Virol., 68:4847-4856, 1994.
Ying and Xiao, 2006
Yoder et al., Blood, 82 (Supp.): 1:347A, 1994.
Yutzey et al. Mol. Cell. Biol., 9:1397, 1989.
Zakut-Houri et al., EMBO J., 4(5):1251-1255, 1985.
Zbar et al., Nature, 327:721-724, 1987.
Zeng et al., Cancer Res., 62(13):3630-3635, 2002.
Zhou et al., Exp. Hematol, 21:928-933, 1993.
Zhou et al., J. Exp. Med., 179:1867-1875, 1994.
Zhu et al., Science, 261(5118):209-211, 1993.
Zunino et al., Biochim. Biophys. Acta, 967(3):331-340, 1988.

1. A method for enhancing the effectiveness of a gene therapy comprising providing a gene therapy and a proteasome inhibitor to a subject in need of the therapy, wherein degradation of a polypeptide gene product of the gene therapy is reduced.

2. The method of claim 1, wherein the gene therapy is a cancer gene therapy.

3. The method of claim 2, wherein the cancer gene therapy is a tumor suppressor gene therapy.

4. The method of claim 3, wherein the tumor suppressor gene therapy is MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1 therapy.

5. The method of claim 3, wherein the tumor suppressor gene therapy is MDA-7 therapy.

6. The method of claim 2, wherein the tumor suppressor gene therapy is p53 gene therapy.

7. The method of claim 1, wherein the proteasome inhibitor is a natural product, a peptide aldehyde, or a boronic acid inhibitor.

8. The method of claim 7, wherein the proteasome inhibitor is lactacystin, MG132, ALLN, MG115, bortezomib or combinations thereof.

9. The method of claim 8, wherein the proteasome inhibitor is MG132.

10. The method of claim 1 wherein the proteasome inhibitor is administered to the patient intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation, by injection, by infusion, by continuous infusion, by localized perfusion bathing target cells directly, via a catheter, or via a lavage.

11. The method of claim 1, wherein the proteasome inhibitor is administered before, after, or during administration of the gene therapy.

12. The method of claim 11, wherein the proteasome inhibitor and gene therapy are formulated together in a composition.

13. A method for treating cancer in a patient comprising providing an effective amount of a tumor suppressor nucleic acid and a proteasome inhibitor to the patient, wherein the tumor suppressor is MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1.

14. The method of claim 12, wherein the proteasome inhibitor is a natural product, a peptide aldehyde, or a boronic acid inhibitor.

15. The method of claim 13, wherein the proteasome inhibitor is lactacystin, MG132, ALLN, MG115, bortezomib or combinations thereof.

16. The method of claim 15, wherein the proteasome inhibitor is MG132.

17.-22. (canceled)

23. The method of claim 13, wherein the nucleic acid is an adenovirus vector.

24.-28. (canceled)

29. The method of claim 13, wherein the patient is provided with a composition comprising the proteasome inhibitor and a nucleic acid having a sequence encoding p53.

30.-35. (canceled)

36. The method of claim 13, wherein the cancer is melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder.

37.-83. (canceled)

84. A pharmaceutical composition comprising:
a) a proteasome inhibitor or proteasome inhibitor prodrug; and
b) an isolated nucleic acid having a sequence encoding a tumor suppressor gene polypeptide;
wherein the tumor suppressor is MDA-7, APC, CYLD, HIN-1, KRAS2b, p16, p19, p21, p27, p27mt, p53, p57, p73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2) or FUS1.

85. The pharmaceutical composition of claim 84, wherein the nucleic acid is an adenovirus vector.

86.-96. (canceled)
